(19) United States
(12) Patent Application Publication
(10) Pub. No.: US 2024/0366761 A1
(43) Pub. Date: Nov. 7, 2024

(54) FUSION PROTEINS AND USES THEREOF

(71) Applicant: Legend Biotech Ireland Limited, Dublin 18 (IE)

(72) Inventors: Xiaohu FAN, Edmonton (CA); Jun WANG, Nanjing (CN); Jie MAO, Nanjing (CN); Ershao ZHANG, Nanjing (CN); Qing WEI, Nanjing (CN); Lian MA, Nanjing (CN)

(21) Appl. No.: 18/284,275

(22) PCT Filed: Apr. 15, 2022
(86) PCT No.: PCT/CN2022/087016

§ 371 Date: Sep. 26, 2023

(30) Foreign Application Priority Data
Apr. 15, 2021 (WO) PCT/CN2021/087475

(51) Int. Cl. A61K 39/00; A61P 35/00; C07K 14/71; C07K 14/715; C07K 14/725; C07K 16/30; C12N 5/0783

(52) U.S. Cl. CPC A61K 39/4631; A61K 39/4611; A61K 39/4613; A61K 39/4632; A61P 35/00; C07K 14/7051; C07K 14/71; C07K 14/7155; C07K 16/30; C07K 16/3007; C07K 16/3069; C07K 16/3092; C12N 5/0636; C07K 2319/03; C12N 2510/00

(57) ABSTRACT

The present application provides fusion proteins that have two polypeptides, each having an extracellular domain and an intracellular domain, and the two extracellular domains form a binding site for TGFβ and the two intracellular domains form the intracellular domain of an IL-23 receptor complex. The present application also provides nucleic acids encoding the fusion proteins, vectors comprising the nucleic acids, and engineered cells expressing the fusion proteins. Method of producing the engineered cells and methods of treatment that involves administering engineered cells are also provided.

### CROSS-REFERENCE TO RELATED APPLICATION

This application claims priority to International application PCT/CN2021/087475, filed on Apr. 15, 2021, the content of which is incorporated by reference in their entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: P11128-PCT.220415.Sequence Listing.TXT, date recorded: Apr. 14, 2022, size: 205,318 bytes).

### FIELD OF THE PRESENT APPLICATION

The present application relates to fusion proteins that bind to TGFβ and are capable of mediating a pro-inflammatory signal.

### BACKGROUND OF THE PRESENT APPLICATION

Transforming growth factor beta (TGFβ) is a pleiotropic cytokine that has been implicated as an immunosuppressive signaling molecule in the tumor microenvironment. TGFβ binds to the TGFβR1 and TGFβR2 serine/threonine kinase receptor complexes, resulting in receptor-mediated phosphorylation of downstream transcription factors Smad2 and Smad3. Many tumors evade the cytostatic and anti-proliferative effects of TGFβ by acquiring mutations in the TGFβ receptors and/or downstream Smad signaling proteins. TGFβ suppresses key molecules involved in the effector and cytolytic activities of T cells in vitro, including IFNγ secretion.

The disclosures of all publications, patents, patent applications and published patent applications referred to herein are hereby incorporated herein by reference in their entirety.

### BRIEF SUMMARY OF THE PRESENT APPLICATION

The present application in aspect provides a fusion protein comprising: a) a first polypeptide comprising i) a first extracellular domain comprising an extracellular domain of one of TGFβR1 or TGFβR2, ii) a first transmembrane domain, and iii) a first intracellular domain comprising an intracellular domain of one of IL-12Rβ1 or IL-23R; and b) a second polypeptide comprising i) a second extracellular domain comprising an extracellular domain of the other of TGFβR1 or TGFβR2, ii) a second transmembrane domain, and iii) a second intracellular domain comprising an intracellular domain of the other of IL-12Rβ1 or IL-23R. In some embodiments, the first transmembrane domain and the second transmembrane domain each comprises a transmembrane domain of one of TGFβR1, TGFβR2, IL-12Rβ1 and IL-23R. In some embodiments, the first transmembrane domain and the second transmembrane domain each comprises a transmembrane domain of one of IL-12Rβ1 or IL-23R.

In some embodiments according to any of the fusion proteins described above, the fusion protein comprises:
a) the first extracellular domain comprises an extracellular domain of TGFβR1, the first transmembrane domain comprises a transmembrane domain of TGFβR1, the first intracellular domain comprises an intracellular domain of IL-23R, the second extracellular domain comprises an extracellular domain of TGFβR2, the second transmembrane domain comprises a transmembrane domain of TGFβR2, and the second intracellular domain comprises an intracellular domain of IL-12Rβ1;
b) the first extracellular domain comprises an extracellular domain of TGFβR2, the first transmembrane domain comprises a transmembrane domain of TGFβR2, the first intracellular domain comprises an intracellular domain of IL-23R, the second extracellular domain comprises an extracellular domain of TGFβR1, the second transmembrane domain comprises a transmembrane domain of TGFβR1, and the second intracellular domain comprises an intracellular domain of IL-12Rβ1;
c) the first extracellular domain comprises an extracellular domain of TGFβR1, the first transmembrane domain comprises a transmembrane domain of IL-23R, the first intracellular domain comprises an intracellular domain of IL-23R, the second extracellular domain comprises an extracellular domain of TGFβR2, the second transmembrane domain comprises a transmembrane domain of IL-12Rβ1, and the second intracellular domain comprises an intracellular domain of IL-12Rβ1; or
d) the first extracellular domain comprises an extracellular domain of TGFβR2, the first transmembrane domain comprises a transmembrane domain of IL-23R, the first intracellular domain comprises an intracellular domain of IL-23R, the second extracellular domain comprises an extracellular domain of TGFβR1, the second transmembrane domain comprises a transmembrane domain of IL-12Rβ1, and the second intracellular domain comprises an intracellular domain of IL-12Rβ1.

In some embodiments according to any of the fusion proteins described above, the first polypeptide and/or the second polypeptide further comprises a signal peptide at the N-terminus of the polypeptide.

In some embodiments according to any of the fusion proteins described above, the first polypeptide and the second polypeptide are in a single polypeptide, and the first polypeptide and the second polypeptide are separated by a multicistronic element. In some embodiments, the multicistronic element comprises a 2A self-cleaving peptide selected from the group consisting of T2A, P2A, E2A, or F2A. In some embodiments, the first polypeptide is N-terminal to the second polypeptide. In some embodiments, the first polypeptide is C-terminal to the second polypeptide. In some embodiments, the single polypeptide comprises, from N-terminus to C-terminus:
a) the first extracellular domain comprising an extracellular domain of TGFβR1, the first transmembrane domain comprising a transmembrane domain of TGFβR1, the first intracellular domain comprising an intracellular domain of IL-23R, a 2A self-cleaving peptide, the second extracellular domain comprising an extracellular domain of TGFβR2, the second transmembrane domain comprising a transmembrane domain of TGFβR2, and the second intracellular domain comprising an intracellular domain of IL-12Rβ1;
b) the first extracellular domain comprising an extracellular domain of TGFβR2, the first transmembrane domain comprising a transmembrane domain of TGFβR2, the first intracellular domain comprising an intracellular domain of IL-23R, a 2A self-cleaving peptide, the second extracellular domain comprising an extracellular domain of TGFβR1, the second transmembrane domain comprising a transmembrane domain of TGFβR1, and the second intracellular domain comprising an intracellular domain of IL-12Rβ1;
c) the first extracellular domain comprising an extracellular domain of TGFβR1, the first transmembrane domain comprising a transmembrane domain of TGFβR1, the first intracellular domain comprising an intracellular domain of IL-12Rβ1, a 2A self-cleaving peptide, the second extracellular domain comprising an extracellular domain of TGFβR2, the second transmembrane domain comprising a transmembrane domain of TGFβR2, and the second intracellular domain comprising an intracellular domain of IL-23R;
d) the first extracellular domain comprising an extracellular domain of TGFβR1, the first transmembrane domain comprising a transmembrane domain of IL-23R, the first intracellular domain comprising an intracellular domain of IL-23R, a 2A self-cleaving peptide, the second extracellular domain comprising an extracellular domain of TGFβR2, the second transmembrane domain comprising a transmembrane domain of IL-12Rβ1, and the second intracellular domain comprising an intracellular domain of IL-12Rβ1;
e) the first extracellular domain comprising an extracellular domain of TGFβR2, the first transmembrane domain comprising a transmembrane domain of IL-23R, the first intracellular domain comprising an intracellular domain of IL-23R, a 2A self-cleaving peptide, the second extracellular domain comprising an extracellular domain of TGFβR1, the second transmembrane domain comprising a transmembrane domain of IL-12Rβ1, and the second intracellular domain comprising an intracellular domain of IL-12Rβ1; or
f) the first extracellular domain comprising an extracellular domain of TGFβR1, the first transmembrane domain comprising a transmembrane domain of IL-12Rβ1, the first intracellular domain comprising an intracellular domain of IL-12Rβ1, a 2A self-cleaving peptide, the second extracellular domain comprising an extracellular domain of TGFβR2, the second transmembrane domain comprising a transmembrane domain of IL-23R, and the second intracellular domain comprising an intracellular domain of IL-23R.

In some embodiments according to any of the fusion proteins described above, the first extracellular domain and the second extracellular domain form a binding site for TGFβ, wherein the first intracellular domain and the second intracellular domain form the intracellular domain of an IL-23 receptor complex, and wherein upon binding of the fusion protein to TGFβ, signaling through the IL-23 receptor complex is transmitted.

In some embodiments according to any of the fusion proteins described above, the transmembrane domain of TGFβR1 comprises the amino acid sequence of SEQ ID NO: 5, or a functional variant having at least about 90% sequence identity; and/or the transmembrane domain of TGFβR2 comprises the amino acid sequence of SEQ ID NO: 6, or a functional variant having at least about 90% sequence identity.

In some embodiments according to any of the fusion proteins described above, the transmembrane domain of IL-23R comprises the amino acid sequence of SEQ ID NO: 7, or a functional variant having at least about 90% sequence identity; and/or the transmembrane domain of IL-12Rβ1 comprises the amino acid sequence of SEQ ID NO: 8, or a functional variant having at least about 90% sequence identity.

In some embodiments according to any of the fusion proteins described above, the extracellular domain of TGFβR1 comprises the amino acid sequence of SEQ ID NO: 3, or a functional variant having at least about 90% sequence identity.

In some embodiments according to any of the fusion proteins described above, the extracellular domain of TGFβR2 comprises the amino acid sequence of SEQ ID NO: 4, or a functional variant having at least about 90% sequence identity.

In some embodiments according to any of the fusion proteins described above, the intracellular domain of IL-23R comprises the amino acid sequence of SEQ ID NO: 15, or a functional variant having at least about 90% sequence identity.

In some embodiments according to any of the fusion proteins described above, the intracellular domain of IL-12Rβ1 comprises the amino acid sequence of SEQ ID NO: 16, or a functional variant having at least about 90% sequence identity.

In some embodiments according to any of the fusion proteins described above, the fusion protein comprises a) a first polypeptide comprising i) a first extracellular domain comprising the amino acid sequence of SEQ ID NO: 3, ii) a first transmembrane domain comprising the amino acid sequence of SEQ ID NO: 5, iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 6, and iii) a second intracellular domain comprising SEQ ID NO: 16.

In some embodiments according to any of the fusion proteins described above, the fusion protein comprises a) a first polypeptide comprising i) a first extracellular domain comprising the amino acid sequence of SEQ ID NO: 4, ii) a first transmembrane domain comprising the amino acid sequence of SEQ ID NO: 6, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 5, and iii) a second intracellular domain comprising the amino acid sequence of SEQ ID NO: 16.

In some embodiments according to any of the fusion proteins described above, the fusion protein comprises a) a first polypeptide comprising i) a first extracellular domain comprising the amino acid sequence of SEQ ID NO: 3, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 8, and iii) a second intracellular domain comprising the amino acid sequence of SEQ ID NO: 16.

In some embodiments according to any of the fusion proteins described above, the fusion protein comprises a) a first polypeptide comprising i) a first extracellular domain comprising the amino acid sequence of SEQ ID NO: 4, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 8, and iii) a second intracellular domain comprising the amino acid sequence of SEQ ID NO: 16.

In some embodiments according to any of the fusion proteins described above, the first polypeptide and/or the second polypeptide further comprises a linker between two domains, wherein one of the two domains is the transmembrane domain, and the other domain is the extracellular domain or the intracellular domain. In some embodiments, the linker comprises a membrane proximal sequence, wherein the membrane proximal sequence and the transmembrane domains are derived from the same molecule. In some embodiments, the membrane proximal region comprises an amino acid sequence set forth in any of SEQ ID NOs: 9-12.

In some embodiments according to any of the fusion proteins described above, the first polypeptide comprises the amino acid sequence of SEQ ID NO: 49, and the second polypeptide comprises the amino acid sequence of SEQ ID NO: 50.

In some embodiments according to any of the fusion proteins described above, the first polypeptide comprises the amino acid sequence of SEQ ID NO: 51, and the second polypeptide comprises the amino acid sequence of SEQ ID NO: 52.

In some embodiments according to any of the fusion proteins described above, the first polypeptide comprises the amino acid sequence of SEQ ID NO: 53, and the second polypeptide comprises the amino acid sequence of SEQ ID NO: 54.

In some embodiments according to any of the fusion proteins described above, the first polypeptide comprises the amino acid sequence of SEQ ID NO: 55, and the second polypeptide comprises the amino acid sequence of SEQ ID NO: 56.

In some embodiments according to any of the fusion proteins described above, the fusion protein comprises an amino acid sequence set forth in any of SEQ ID NOs: 20-25.

The present application in another aspect provides a nucleic acid comprising one or more nucleic acid sequences encoding any of the fusion proteins described above or a portion thereof.

The present application in another aspect provides a nucleic acid comprising a nucleic acid sequence set forth in any of SEQ ID NOs: 28-33.

In some embodiments according to any of the nucleic acids described above, the nucleic acid further comprises a second nucleic acid sequence encoding a functional exogenous receptor, wherein the functional exogenous receptor comprises an extracellular ligand-binding domain and optionally an intracellular signaling domain. In some embodiments, the functional exogenous receptor is selected from the group consisting of: an engineered T cell receptor (TCR), a chimeric antigen receptor (CAR), a T cell antigen coupler (TAC) or a portion thereof. In some embodiments, the functional exogenous receptor specifically recognizes a tumor antigen. In some embodiments, the nucleic acid sequence encoding the functional exogenous receptor is upstream to at least one of the one or more nucleic acid sequences encoding the fusion protein (“fusion protein nucleic acid sequence”), and optionally wherein functional exogenous receptor nucleic acid sequence and the fusion protein nucleic acid sequence are separated by a third nucleic acid sequence encoding a second multicistronic element. In some embodiments, the second multicistronic element comprises a 2A self-cleaving peptide selected from the group consisting of T2A, P2A, E2A, or F2A. In some embodiments, the nucleic acid sequence encoding the fusion protein and nucleic acid sequence encoding the functional exogenous receptor are separated by the second multicistronic element.

The present application in another aspect provides a vector comprising any of the nucleic acids described above.

The present application in another aspect provides an engineered cell, comprising any of the fusion protein, the nucleic acid or the vector described above. In some embodiments, the engineered cell further comprises a functional exogenous receptor, wherein the functional exogenous receptor comprises an extracellular ligand-binding domain and optionally an intracellular signaling domain. In some embodiments, the functional exogenous receptor is selected from the group consisting of: an engineered T cell receptor (TCR), a chimeric antigen receptor (CAR), a T cell antigen coupler (TAC) or a portion thereof. In some embodiments, the functional exogenous receptor specifically recognizes a tumor antigen. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof.

The present application in another aspect provides a pharmaceutical composition comprising any of the engineered cells described above.

The present application in another aspect provides a method of treating a disease or condition in an individual, comprising administering to the individual any of the fusion proteins or the pharmaceutical compositions described herein. In some embodiments, the disease or condition is associated with immunosuppression. In some embodiments, the diseased tissue has a higher expression level of TGFβ than a corresponding tissue in an individual without the disease or condition. In some embodiments, the disease or condition is a cancer. In some embodiments, the cancer is a solid tumor. In some embodiments, the disease or condition is an infectious disease or a condition associated with an infection. In some embodiments, the engineered cells in the pharmaceutical composition are allogenic to the individual. In some embodiments, the engineered cells in the pharmaceutical composition are autologous to the individual. In some embodiments, the method further comprises a second therapy or administering a second agent.

The present application in another aspect provides a method of reducing an immunosuppression signal in a diseased tissue in an individual, comprising administering to the individual any of the fusion proteins or the pharmaceutical compositions described herein. In some embodiments, the reducing the immunosuppression signal comprises decreasing signaling through TGFβR. In some embodiments, the diseased tissue has a higher expression level of TGFβ than a corresponding tissue in an individual without the disease or condition. In some embodiments, the disease or condition is a cancer. In some embodiments, the cancer is a solid tumor. In some embodiments, the disease or condition is an infectious disease or a condition associated with an infection. In some embodiments, the engineered cells in the pharmaceutical composition are allogenic to the individual. In some embodiments, the engineered cells in the pharmaceutical composition are autologous to the individual. In some embodiments, the method further comprises a second therapy or administering a second agent.

### BRIEF DESCRIPTION OF THE DRAWINGS

FIGS. 1A-1B show cartoon structures of TGB23 fusion proteins (FIG. 1A) and polypeptides encoding TGB23 fusion proteins (FIG. 1B). SP refers to the signal peptide. TM refers to the transmembrane domain.

FIG. 2 shows the expression levels of fusion proteins TGB23-1, TGB23-2, TGB23-3, TGB23-4, TGB23-5, TGB23-6, wtTGFβR, TGB23-14, and TGB23-15. UnT refers to T cells un-transduced with any of the fusion proteins, and wtTGFβR refers to wild type TGFβ Receptor.

FIGS. 3A-3B show the cell viability (FIG. 3A) and cell number (FIG. 3B) of T cells expressing TGB23-1, TGB23-2, TGB23-3, TGB23-4, TGB23-5, TGB23-6, wtTGFβR, TGB23-14 or TGB23-15 treated with 20 ng/mL TGFβ1. UnT refers to T cells un-transduced with any of the fusion proteins.

FIG. 4 shows the anti-GPC3 CAR expression level and TGFβR2 expression level in CAR positive cells of UnT, huLIC19309b CAR-T cells and huLIC19309bT CAR-T cells. UnT refers to T cells un-transduced with CAR.

FIG. 5 shows the expression level of pSmad2 in UnT, huLIC19309b CAR-T cells and huLIC19309bT CAR-T cells treated with or without 20 ng/mL TGFβ1 for 4 hours. UnT refers to T cells un-transduced with CAR.

FIGS. 6A-6B show the expression level of pStat3 (FIG. 6A) and pStat4 (FIG. 6B) in UnT, huLIC19309b CAR-T cells and huLIC19309bT CAR-T cells treated with or without 20 ng/mL TGFβ1 for 4 hours. UnT refers to T cells un-transduced with CAR.

FIGS. 7A-7B show the cell viability (FIG. 7A) and cell number (FIG. 7B) of UnT, huLIC19309b CAR-T cells and huLIC19309bT CAR-T cells treated with 20 ng/mL TGFβ1. UnT refers to T cells un-transduced with CAR.

FIGS. 8A-8B show the expression level of PD1 in CD4+CAR+ T cells (FIG. 8A) and CD8+CAR+ T cells (FIG. 8B) of the two treatment groups (huLIC19309b CAR-T group and huLIC19309bT CAR-T group) after co-culture with PLCPRF5 and 5 ng/mL TGFβ1 in re-challenge assay.

FIGS. 9A-9D show the CAR positive ratio (FIG. 9A and FIG. 9B) and cell number (FIG. 9C and FIG. 9D) after co-culture with PLCPRF5 in re-challenge assay.

FIGS. 10A-10B show in vitro cytotoxicity of huLIC19309b CAR-T cells and huLIC19309bT CAR-T cells against PLCPRF5 (FIG. 10A) and Hep3B2.1-7 cells (FIG. 10B) with E/T ratio of 1:1 in re-challenge assay. E/T refers to the ratio of effective cell to target cell.

FIG. 11 shows the real time cytotoxicity of huLIC19309b CAR-T cells and huLIC19309bT CAR-T cells against Hep3B2.1-7 cells with E/T ratio of 1:20 in vitro. E/T refers to the ratio of effective cell to target cell.

FIGS. 12A-12C show the anti-tumor effects of CAR-T cells in NCG mouse xenograft model. NCG mice were inoculated subcutaneously with Hep3B2.1-7 cells, and treated with CAR-T cells at 0.3 M dosage and 0.8 M dosage (i.v.). The mouse body weight (FIG. 12A), volume of tumors (FIG. 12B) and the percentage of human CD3+ T cells in peripheral blood (FIG. 12C) were assessed.

FIGS. 13A-13C show the expression of TCRαβ (FIG. 13A), CD20 CAR (FIG. 13B) and TGB23-6 (FIG. 13C) of UnT, LCAR-UL186S T cells and LCAR-UL186S+TGB23-6 T cells by flow cytometry. Un-transduced T cells (UnT) were used as a negative control.

FIG. 14 shows in vitro cytotoxicity of LCAR-UL186S T cells and LCAR-UL186S+TGB23-6 T cells against Raji cells with different effector-target ratios. E/T refers to the ratio of effective cell to target cell.

FIG. 15 shows the proliferation of LCAR-UL186S T cells and LCAR-UL186S+TGB23-6 T cells after multiple rounds of Raji stimulation. Cells were harvested at day0, day4, day7, day11 and the expression of CD5 was detected by flow cytometry.

### DETAILED DESCRIPTION OF THE PRESENT APPLICATION

The present application provides fusion proteins a) a first polypeptide comprising i) a first extracellular domain comprising an extracellular domain of one of TGFβR1 or TGFβR2, ii) a first transmembrane domain, and iii) a first intracellular domain comprising an intracellular domain of one of IL-12Rβ1 or IL-23R; and b) a second polypeptide comprising i) a second extracellular domain comprising an extracellular domain of the other of TGFβR1 or TGFβR2, ii) a second transmembrane domain, and iii) a second intracellular domain comprising an intracellular domain of the other of IL-12Rβ1 or IL-23R. In some embodiments, the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted. The present application also provides engineered cells comprising such fusion protein, or a nucleic acid encoding such a fusion protein, optionally the engineered cells also comprise a functional exogenous receptor (e.g., a CAR or an engineered TCR). Methods of producing or using such engineered cells are also provided.

The present application provides that engineered cells comprising a fusion protein described herein are able to transform a TGFβ signal to an IL-23R mediated signal. As shown in more details in the examples, engineered T cells that have such fusion protein exhibit a higher viability and proliferation than corresponding T cells without the fusion protein upon exposure to TGFβ. Moreover, engineered T cells that express both the fusion protein and a CAR that targets a tumor antigen exhibit higher cytotoxicity against tumor cells both in vitro and in an animal tumor model. It has also been shown that the increased proliferation and cytotoxicity of the engineered T cells pertain throughout serial exposures to TGFβ that last more than a total of 300 hours. These results provide strong evidence that fusion proteins and engineered cells comprising these fusion proteins can effectively treat diseases or conditions associated with immunosuppression (such as a cancer). These highly advantageous effects are particularly striking in view of the currently limited and conflicting understanding of IL-23 signaling in cancer biology.

### I. Definitions

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)₂, and Fv). The term “immunoglobulin” (Ig) is used interchangeably with “antibody” herein. Antibodies contemplated herein include single-domain antibodies, such as heavy chain only antibodies.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H}Hs”. The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs. Some V_{H}Hs may also be known as Nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H}H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “V_{H}H”.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) in both the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

“Fv” is the minimum antibody fragment, which contains a complete antigen-recognition and -binding site. This fragment consists of a dimer of one heavy- and one light-chain variable region domain in tight, non-covalent association. From the folding of these two domains, emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains, which enables the sFv to form the desired structure for antigen binding.

As use herein, the term “specifically binds,” “specifically recognizes,” or is “specific for” refers to measurable and reproducible interactions such as binding between a target and an antigen binding protein (such as an antigen-binding domain, a ligand, an engineered TCR, a CAR, or a chimeric receptor), which is determinative of the presence of the target in the presence of a heterogeneous population of molecules including biological molecules. For example, an antigen binding protein that specifically binds a target (which can be an epitope) is an antigen binding protein that binds this target with greater affinity, avidity, more readily, and/or with greater duration than it binds other targets. In some embodiments, the extent of binding of an antigen binding protein to an unrelated target is less than about 10% of the binding of the antigen binding protein to the target as measured, e.g., by a radioimmunoassay (RIA). In some embodiments, an antigen binding protein that specifically binds a target has a dissociation constant (Kd) of ≤1 μM, ≤100 nM, ≤10 nM, ≤1 nM, or ≤0.1 nM. In some embodiments, an antigen binding protein specifically binds an epitope on a protein that is conserved among the protein from different species. In some embodiments, specific binding can include, but does not require exclusive binding.

The term “specificity” refers to selective recognition of an antigen binding protein (such as a CAR or an antibody) for a particular epitope of an antigen. Natural antibodies, for example, are monospecific. The term “multispecific” as used herein denotes that an antigen binding protein (such as a CAR or an antibody) has two or more antigen-binding sites of which at least two bind different antigens. “Bispecific” as used herein denotes that an antigen binding protein (such as a CAR or an antibody) has two different antigen-binding specificities. The term “monospecific” as used herein denotes an antigen binding protein (such as a CAR or an antibody) that has one or more binding sites each of which bind the same antigen.

The term “valent” as used herein denotes the presence of a specified number of binding sites in an antigen binding protein (such as a CAR or an antibody). A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein (such as a CAR or an antibody).

The term “extracellular domain” refers to the fragment or portion of a membrane receptor on the outside of the cell. The extracellular domain is or includes the ligand binding or recognition domain (e.g., TGFβ binding domain of TGFβR1 and TGFβR2 in this application).

The term “intracellular domain” refers to the portion of a protein which transduces the effector function signal and that directs the cell to perform a specialized function. While usually the entire intracellular domain can be employed, in some cases it is not necessary to use the entire domain. To the extent that a truncated portion of an intracellular domain is used, such truncated portion may be used in place of the entire domain as long as it transduces the effector function signal. The term intracellular domain is meant to include any truncated portion of the intracellular signaling domain sufficient to transducing effector function signal.

The term “transmembrane domain” or “TM domain” is a domain that anchors a polypeptide to the plasma membrane of a cell. The TM domain may be derived either from a natural, synthetic, semi-synthetic, or recombinant source. The transmembrane domain may be from the same receptor molecule of either the extracellular domain or the intracellular domain.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Binding affinity” generally refers to the strength of the sum total of non-covalent interactions between a single binding site of a molecule (e.g., an antibody, a CAR) and its binding partner (e.g., an antigen). Unless indicated otherwise, as used herein, “binding affinity” refers to intrinsic binding affinity that reflects a 1:1 interaction between members of a binding pair (e.g., antibody and antigen, or CAR and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity is known in the art, any of which can be used for purposes of the present application. Specific illustrative and exemplary embodiments for measuring binding affinity are described in the following.

“Percent (%) sequence identity” and “homology” with respect to a peptide, a polypeptide or a nucleic acid are defined as the percentage of amino acid residues or nucleotides in a candidate sequence that are identical with the amino acid residues or nucleotides in the specific peptide, polypeptide, or nucleic acid sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen-binding domain specific for one or more antigens (such as tumor antigens), a transmembrane domain, and an intracellular signaling domain of a T cell and/or other receptors. “CAR-T” refers to a T cell that expresses a CAR. “GPC3 CAR” refers to a CAR having an extracellular binding domain specific for GPC3. “Bi-epitope CAR” refers to a CAR having an extracellular binding domain specific for two different epitopes.

An “isolated” nucleic acid molecule (e.g., encoding a fusion protein, a CAR, or an engineered TCR) described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Unless otherwise specified, a “nucleic acid sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present application contemplate any one or more of these aspects of treatment.

As used herein, an “individual” or a “subject” refers to a mammal, including, but not limited to, human, bovine, horse, feline, canine, rodent, or primate. In some embodiments, the individual is a human.

The term “effective amount” used herein refers to an amount of an agent, such as an engineered T cell described herein, or a pharmaceutical composition thereof, sufficient to treat a specified disorder, condition or disease such as ameliorate, palliate, lessen, and/or delay one or more of its symptoms (e.g., cancer or infectious disease). In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the agent (e.g., engineered T cell) or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. In the case of infectious disease, such as viral infection, the therapeutically effective amount of an engineered T cell described herein or composition thereof can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

As used herein, “delaying” the development of a disease means to defer, hinder, slow, retard, stabilize, and/or postpone development of the disease (e.g., cancer or infectious disease). This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

As used herein, the term “autologous” is meant to refer to any material derived from the same individual to whom it is later to be re-introduced into the individual.

“Allogeneic” refers to a graft derived from a different individual of the same species. “Allogeneic T cell” refers to a T cell from a donor having a tissue human leukocyte antigen (HLA) type that matches the recipient. Typically, matching is performed based on variability at three or more loci of the HLA gene, and a perfect match at these loci is preferred. In some instances allogeneic transplant donors may be related (usually a closely HLA matched sibling), syngeneic (a monozygotic “identical” twin of the patient) or unrelated (donor who is not related and found to have very close degree of HLA matching). The HLA genes fall in two categories (Type I and Type II). In general, mismatches of the Type-I genes (i.e., HLA-A, HLA-B, or HLA-C) increase the risk of graft rejection. A mismatch of an HLA Type II gene (i.e., HLA-DR, or HLA-DQB1) increases the risk of graft-versus-host disease (GvHD).

A “patient” as used herein includes any human who is afflicted with a disease (e.g., cancer, viral infection). The terms “subject,” “individual,” and “patient” are used interchangeably herein. The term “donor subject” or “donor” refers to herein a subject whose cells are being obtained for further in vitro engineering. The donor subject can be a patient that is to be treated with a population of cells generated by the methods described herein (i.e., an autologous donor), or can be an individual who donates a blood sample (e.g., lymphocyte sample) that, upon generation of the population of cells generated by the methods described herein, will be used to treat a different individual or patient (i.e., an allogeneic donor). Those subjects who receive the cells that were prepared by the present methods can be referred to as “recipient” or “recipient subject.”

The term “T cell receptor,” or “TCR,” refers to a heterodimeric receptor composed of αβ or γδ chains that pair on the surface of a T cell. Each α, β, γ, and δ chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (pMIC) (Davis and Bjorkman (1988) Nature, 334, 395-402; Davis et al. (1998) Annu Rev Immunol, 16, 523-544; Murphy (2012), xix, 868 p.).

The term “stimulation”, as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and upregulate or downregulate expression or secretion of a molecule, such as downregulation of TGF-β. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The term “activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process. The term “activated T cells” indicates T cells that are currently undergoing cell division, cytokine production, performance of reg. or cytol. Effector functions, and/or has recently undergone the process of “activation.”

The term “functional exogenous receptor” as used herein, refers to an exogenous receptor (e.g., CAR, engineered receptor, engineered TCR, or antibody coupled TCR (ACTR)) that retains its biological activity after being introduced into the T cells or fusion protein-expressing T cell described herein. The biological activity include but are not limited to the ability of the exogenous receptor in specifically binding to a molecule (e.g., cancer antigen, or an antibody for ACTR), properly transducing downstream signals, such as inducing cellular proliferation, cytokine production and/or performance of regulatory or cytolytic effector functions.

It is understood that embodiments of the present application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y”.

As used herein and in the appended claims, the singular forms “a,” “or,” and “the” include plural referents unless the context clearly dictates otherwise.

### II. Fusion Proteins of the Present Invention

The present application in one aspect provides fusion proteins comprising: a) a first polypeptide comprising i) a first extracellular domain comprising an extracellular domain of one of TGFβR1 or TGFβR2, ii) a first transmembrane domain, and iii) a first intracellular domain comprising an intracellular domain of one of IL-12Rβ1 or IL-23R; and b) a second polypeptide comprising i) a second extracellular domain comprising an extracellular domain of the other of TGFβR1 or TGFβR2, ii) a second transmembrane domain, and iii) a second intracellular domain comprising an intracellular domain of the other of IL-12Rβ1 or IL-23R.

In some embodiments, there is provided a fusion protein comprising a) a first polypeptide comprising i) a first extracellular domain comprising an extracellular domain of TGFβR1, ii) a first transmembrane domain comprising a transmembrane domain of TGFβR1, iii) a first intracellular domain comprising an intracellular domain of IL-23R; and b) a second polypeptide comprising i) a second extracellular domain comprising an extracellular domain of TGFβR2, ii) a second transmembrane domain comprising a transmembrane domain of TGFβR2, and iii) a second intracellular domain comprising an intracellular domain of IL-12Rβ1. In some embodiments, the extracellular domain of TGFβR1 comprises the amino acid sequence of SEQ ID NO: 3, or a functional variant having at least about 80% (such as at least about any one of 80%, 85%, 90%, 95%, 96%, 97%, 98%, or 99%) sequence identity. In some embodiments, the extracellular domain of TGFβR2 comprises the amino acid sequence of SEQ ID NO: 4, or a functional variant having at least about 80% (such as at least about any one of 80%, 85%, 90%, 95%, 96%, 97%, 98%, or 99%) sequence identity. In some embodiments, the intracellular domain of IL-23R comprises the amino acid sequence of SEQ ID NO: 15, or a functional variant having at least about 80% (such as at least about any one of 80%, 85%, 90%, 95%, 96%, 97%, 98%, or 99%) sequence identity. In some embodiments, the intracellular domain of IL-12Rβ1 comprises the amino acid sequence of SEQ ID NO: 16, or a functional variant having at least about 80% sequence identity (such as at least about any one of 80%, 85%, 90%, 95%, 96%, 97%, 98%, or 99%). In some embodiments, the transmembrane domain of TGFβR1 comprises the amino acid sequence of SEQ ID NO: 5, or a functional variant having at least about 80% (such as at least about any one of 80%, 85%, 90%, 95%, 96%, 97%, 98%, or 99%) sequence identity. In some embodiments, the transmembrane domain of TGFβR2 comprises the amino acid sequence of SEQ ID NO: 6, or a functional variant having at least about 80% (such as at least about any one of 80%, 85%, 90%, 95%, 96%, 97%, 98%, or 99%) sequence identity. In some embodiments, the extracellular domain of TGFβR1 or TGFβR2 is a mutant or truncated extracellular domain of TGFβR1 or TGFβR2. In some embodiments, the intracellular domain of IL-12Rβ1 or IL-23R is a mutant or truncated intracellular domain of IL-12Rβ1 or IL-23R. In some embodiments, the mutant or truncated intracellular domain of IL-23R has the amino acid sequence of SEQ ID NO: 17 or 18, or a variant having at least about 80% (such as at least about any one of 80%, 85%, 90%, 95%, 96%, 97%, 98%, or 99%) sequence identity. In some embodiments, the truncated intracellular domain of IL-12Rβ1 has the amino acid sequence of SEQ ID NO: 19, or a variant having at least about 80% (such as at least about any one of 80%, 85%, 90%, 95%, 96%, 97%, 98%, or 99%) sequence identity. In some embodiments, the first polypeptide and/or the second polypeptide further comprises a signal peptide at the N-terminus of the polypeptide. In some embodiments, the signal peptide is a signal peptide from TGFβR1 or TGFβR2, optionally the signal peptide and the extracellular domain in the polypeptide are derived from the same molecule. In some embodiments, the signal peptide has the amino acid sequence of SEQ ID NO: 1 or 2. In some embodiments, the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted. In some embodiments, the first polypeptide and/or the second polypeptide further comprises a membrane proximal peptide between the transmembrane domain and the intracellular domain. In some embodiments, the membrane proximal peptide has a length of about 3 to about 40 amino acids. In some embodiments, the membrane proximal peptide is derived from a membrane proximal region of TGFβR1, TGFβR2, IL-23R, or IL-12Rβ1. In some embodiments, the membrane proximal peptide comprises an amino acid sequence set forth in any of SEQ ID NOs: 9-12. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising an extracellular domain of TGFβR2, ii) a first transmembrane domain comprising a transmembrane domain of TGFβR2, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of TGFβR1, and iii) a second intracellular domain comprising an intracellular domain of IL-12Rβ1. In some embodiments, the extracellular domain of TGFβR1 comprises the amino acid sequence of SEQ ID NO: 3, or a functional variant having at least about 80% (such as at least about any one of 80%, 85%, 90%, 95%, 96%, 97%, 98%, or 99%) sequence identity. In some embodiments, the extracellular domain of TGFβR2 comprises the amino acid sequence of SEQ ID NO: 4, or a functional variant having at least about 80% (such as at least about any one of 80%, 85%, 90%, 95%, 96%, 97%, 98%, or 99%) sequence identity. In some embodiments, the intracellular domain of IL-23R comprises the amino acid sequence of SEQ ID NO: 15, or a functional variant having at least about 80% (such as at least about any one of 80%, 85%, 90%, 95%, 96%, 97%, 98%, or 99%) sequence identity. In some embodiments, the intracellular domain of IL-12Rβ1 comprises the amino acid sequence of SEQ ID NO: 16, or a functional variant having at least about 80% sequence identity (such as at least about any one of 80%, 85%, 90%, 95%, 96%, 97%, 98%, or 99%). In some embodiments, the transmembrane domain of TGFβR1 comprises the amino acid sequence of SEQ ID NO: 5, or a functional variant having at least about 80% (such as at least about any one of 80%, 85%, 90%, 95%, 96%, 97%, 98%, or 99%) sequence identity. In some embodiments, the transmembrane domain of TGFβR2 comprises the amino acid sequence of SEQ ID NO: 6, or a functional variant having at least about 80% (such as at least about any one of 80%, 85%, 90%, 95%, 96%, 97%, 98%, or 99%) sequence identity. In some embodiments, the extracellular domain of TGFβR1 or TGFβR2 is a mutant or truncated extracellular domain of TGFβR1 or TGFβR2. In some embodiments, the intracellular domain of IL-12Rβ1 or IL-23R is a mutant or truncated intracellular domain of IL-12Rβ1 or IL-23R. In some embodiments, the mutant or truncated intracellular domain of IL-23R has the amino acid sequence of SEQ ID NO: 17 or 18, or a variant having at least about 80% (such as at least about any one of 80%, 85%, 90%, 95%, 96%, 97%, 98%, or 99%) sequence identity. In some embodiments, the truncated intracellular domain of IL-12Rβ1 has the amino acid sequence of SEQ ID NO: 19, or a variant having at least about 80% (such as at least about any one of 80%, 85%, 90%, 95%, 96%, 97%, 98%, or 99%) sequence identity. In some embodiments, the first polypeptide and/or the second polypeptide further comprises a signal peptide at the N-terminus of the polypeptide. In some embodiments, the signal peptide is a signal peptide from TGFβR1 or TGFβR2, optionally the signal peptide and the extracellular domain in the polypeptide are derived from the same molecule. In some embodiments, the signal peptide has the amino acid sequence of SEQ ID NO: 1 or 2. In some embodiments, the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted. In some embodiments, the first polypeptide and/or the second polypeptide further comprises a membrane proximal peptide between the transmembrane domain and the intracellular domain. In some embodiments, the membrane proximal peptide has a length of about 3 to about 40 amino acids. In some embodiments, the membrane proximal peptide is derived from a membrane proximal region of TGFβR1, TGFβR2, IL-23R, or IL-12Rβ1. In some embodiments, the membrane proximal peptide comprises an amino acid sequence set forth in any of SEQ ID NOs: 9-12. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising an extracellular domain of TGFβR1, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR2, ii) a second transmembrane domain comprising a transmembrane domain of IL-12Rβ1, and iii) a second intracellular domain comprising an intracellular domain of IL-12Rβ1. In some embodiments, the extracellular domain of TGFβR1 comprises the amino acid sequence of SEQ ID NO: 3, or a functional variant having at least about 80% (such as at least about any one of 80%, 85%, 90%, 95%, 96%, 97%, 98%, or 99%) sequence identity. In some embodiments, the extracellular domain of TGFβR2 comprises the amino acid sequence of SEQ ID NO: 4, or a functional variant having at least about 80% (such as at least about any one of 80%, 85%, 90%, 95%, 96%, 97%, 98%, or 99%) sequence identity. In some embodiments, the intracellular domain of IL-23R comprises the amino acid sequence of SEQ ID NO: 15, or a functional variant having at least about 80% (such as at least about any one of 80%, 85%, 90%, 95%, 96%, 97%, 98%, or 99%) sequence identity. In some embodiments, the intracellular domain of IL-12Rβ1 comprises the amino acid sequence of SEQ ID NO: 16, or a functional variant having at least about 80% sequence identity (such as at least about any one of 80%, 85%, 90%, 95%, 96%, 97%, 98%, or 99%). In some embodiments, the transmembrane domain of IL-23R comprises the amino acid sequence of SEQ ID NO: 7, or a functional variant having at least about 80% (such as at least about any one of 80%, 85%, 90%, 95%, 96%, 97%, 98%, or 99%) sequence identity. In some embodiments, the transmembrane domain of IL-12Rβ1 comprises the amino acid sequence of SEQ ID NO: 8, or a functional variant having at least about 80% (such as at least about any one of 80%, 85%, 90%, 95%, 96%, 97%, 98%, or 99%) sequence identity. In some embodiments, the extracellular domain of TGFβR1 or TGFβR2 is a mutant or truncated extracellular domain of TGFβR1 or TGFβR2. In some embodiments, the intracellular domain of IL-12Rβ1 or IL-23R is a mutant or truncated intracellular domain of IL-12Rβ1 or IL-23R. In some embodiments, the mutant or truncated intracellular domain of IL-23R has the amino acid sequence of SEQ ID NO: 17 or 18, or a variant having at least about 80% (such as at least about any one of 80%, 85%, 90%, 95%, 96%, 97%, 98%, or 99%) sequence identity. In some embodiments, the truncated intracellular domain of IL-12Rβ1 has the amino acid sequence of SEQ ID NO: 19, or a variant having at least about 80% (such as at least about any one of 80%, 85%, 90%, 95%, 96%, 97%, 98%, or 99%) sequence identity. In some embodiments, the first polypeptide and/or the second polypeptide further comprises a signal peptide at the N-terminus of the polypeptide. In some embodiments, the signal peptide is a signal peptide from TGFβR1 or TGFβR2, optionally the signal peptide and the extracellular domain in the polypeptide are derived from the same molecule. In some embodiments, the signal peptide has the amino acid sequence of SEQ ID NO: 1 or 2. In some embodiments, the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted. In some embodiments, the first polypeptide and/or the second polypeptide further comprises a membrane proximal peptide between the extracellular domain and the transmembrane domain. In some embodiments, the membrane proximal peptide has a length of about 3 to about 40 amino acids. In some embodiments, the membrane proximal peptide is derived from a membrane proximal region of TGFβR1, TGFβR2, IL-23R, or IL-12Rβ1. In some embodiments, the membrane proximal peptide comprises an amino acid sequence set forth in any of SEQ ID NOs: 9-12. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1. In some embodiments, the extracellular domain of TGFβR1 comprises the amino acid sequence of SEQ ID NO: 3, or a functional variant having at least about 80% (such as at least about any one of 80%, 85%, 90%, 95%, 96%, 97%, 98%, or 99%) sequence identity. In some embodiments, the extracellular domain of TGFβR2 comprises the amino acid sequence of SEQ ID NO: 4, or a functional variant having at least about 80% (such as at least about any one of 80%, 85%, 90%, 95%, 96%, 97%, 98%, or 99%) sequence identity. In some embodiments, the intracellular domain of IL-23R comprises the amino acid sequence of SEQ ID NO: 15, or a functional variant having at least about 80% (such as at least about any one of 80%, 85%, 90%, 95%, 96%, 97%, 98%, or 99%) sequence identity. In some embodiments, the intracellular domain of IL-12Rβ1 comprises the amino acid sequence of SEQ ID NO: 16, or a functional variant having at least about 80% sequence identity (such as at least about any one of 80%, 85%, 90%, 95%, 96%, 97%, 98%, or 99%). In some embodiments, the transmembrane domain of IL-23R comprises the amino acid sequence of SEQ ID NO: 7, or a functional variant having at least about 80% (such as at least about any one of 80%, 85%, 90%, 95%, 96%, 97%, 98%, or 99%) sequence identity. In some embodiments, the transmembrane domain of IL-12Rβ1 comprises the amino acid sequence of SEQ ID NO: 8, or a functional variant having at least about 80% (such as at least about any one of 80%, 85%, 90%, 95%, 96%, 97%, 98%, or 99%) sequence identity. In some embodiments, the extracellular domain of TGFβR1 or TGFβR2 is a mutant or truncated extracellular domain of TGFβR1 or TGFβR2. In some embodiments, the intracellular domain of IL-12Rβ1 or IL-23R is a mutant or truncated intracellular domain of IL-12Rβ1 or IL-23R. In some embodiments, the mutant or truncated intracellular domain of IL-23R has the amino acid sequence of SEQ ID NO: 17 or 18, or a variant having at least about 80% (such as at least about any one of 80%, 85%, 90%, 95%, 96%, 97%, 98%, or 99%) sequence identity. In some embodiments, the truncated intracellular domain of IL-12Rβ1 has the amino acid sequence of SEQ ID NO: 19, or a variant having at least about 80% (such as at least about any one of 80%, 85%, 90%, 95%, 96%, 97%, 98%, or 99%) sequence identity. In some embodiments, the first polypeptide and/or the second polypeptide further comprises a signal peptide at the N-terminus of the polypeptide. In some embodiments, the signal peptide is a signal peptide from TGFβR1 or TGFβR2, optionally the signal peptide and the extracellular domain in the polypeptide are derived from the same molecule. In some embodiments, the signal peptide has the amino acid sequence of SEQ ID NO: 1 or 2. In some embodiments, the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted. In some embodiments, the first polypeptide further comprises a first membrane proximal sequence between the first extracellular domain and the first transmembrane domain. In some embodiments, the second polypeptide further comprises a second membrane proximal sequence between the second extracellular domain and the second transmembrane domain. The membrane proximal sequence may serve as a transition between two function domains to provide flexibility and/or promote lifespan of the fusion protein or signal transaction. In some embodiments, the first or the second membrane proximal domain and the first or the second transmembrane domain in the first and/or second polypeptide are derived from the same molecule. In some embodiments, the first and/or second membrane proximal peptide is derived from a membrane proximal region of TGFβR1, TGFβR2, IL-23R, or IL-12Rβ1. In some embodiments, the first and/or second membrane proximal domain has a length of about 3 to about 40 amino acids. In some embodiments, the membrane proximal peptide comprises an amino acid sequence set forth in any of SEQ ID NOs: 9-12. In some embodiments, the first membrane proximal peptide is derived from IL-23R (e.g., SEQ ID NO: 11), and/or the second proximal peptide is derived from IL-12Rβ1 (e.g., SEQ ID NO: 12). In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising the amino acid sequence of SEQ ID NO: 3, or a functional variant having at least about 80% (such as at least about any one of 80%, 85%, 90%, 95%, 96%, 97%, 98%, or 99%) sequence identity, ii) a first transmembrane domain comprising the amino acid sequence of SEQ ID NO: 5, or a variant having at least about 80% (such as at least about any one of 80%, 85%, 90%, 95%, 96%, 97%, 98%, or 99%) sequence identity, iii) a first intracellular domain comprising the amino acid sequence of SEQ ID NO: 15, or a functional variant having at least about 80% (such as at least about any one of 80%, 85%, 90%, 95%, 96%, 97%, 98%, or 99%) sequence identity; and b) a second polypeptide comprising i) a second extracellular domain comprising the amino acid sequence of SEQ ID NO: 4, or a functional variant having at least about 80% (such as at least about any one of 80%, 85%, 90%, 95%, 96%, 97%, 98%, or 99%) sequence identity, ii) a second transmembrane domain comprising the amino acid sequence of SEQ ID NO: 6, or a variant having at least about 80% (such as at least about any one of 80%, 85%, 90%, 95%, 96%, 97%, 98%, or 99%) sequence identity, and iii) a second intracellular domain comprising SEQ ID NO: 16, or a functional variant having at least about 80% (such as at least about any one of 80%, 85%, 90%, 95%, 96%, 97%, 98%, or 99%) sequence identity. In some embodiments, the first polypeptide further comprises a linker (e.g., a membrane proximal sequence) between the first transmembrane domain and the first intracellular domain, optionally the linker comprises the amino acid sequence of SEQ ID NO: 9, or a variant having at least about 80% (such as at least about any one of 80%, 85%, 90%, 95%, 96%, 97%, 98%, or 99%) sequence identity. In some embodiments, the second polypeptide further comprises a linker (e.g., a membrane proximal sequence) between the second transmembrane domain and the second intracellular domain, optionally the linker comprises the amino acid sequence of SEQ ID NO: 10, or a variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising the amino acid sequence of SEQ ID NO: 4, or a functional variant having at least about 80% (such as at least about any one of 80%, 85%, 90%, 95%, 96%, 97%, 98%, or 99%) sequence identity, ii) a first transmembrane domain comprising the amino acid sequence of SEQ ID NO: 6, or a variant having at least about 80% (such as at least about any one of 80%, 85%, 90%, 95%, 96%, 97%, 98%, or 99%) sequence identity, and iii) a first intracellular domain comprising the amino acid sequence of SEQ ID NO: 15, or a functional variant having at least about 80% (such as at least about any one of 80%, 85%, 90%, 95%, 96%, 97%, 98%, or 99%) sequence identity; and b) a second polypeptide comprising i) a second extracellular domain comprising the amino acid sequence of SEQ ID NO: 3, or a functional variant having at least about 80% (such as at least about any one of 80%, 85%, 90%, 95%, 96%, 97%, 98%, or 99%) sequence identity, ii) a second transmembrane domain comprising the amino acid sequence of SEQ ID NO: 5, or a variant having at least about 80% (such as at least about any one of 80%, 85%, 90%, 95%, 96%, 97%, 98%, or 99%) sequence identity, and iii) a second intracellular domain comprising the amino acid sequence of SEQ ID NO: 16, or a functional variant having at least about 80% (such as at least about any one of 80%, 85%, 90%, 95%, 96%, 97%, 98%, or 99%) sequence identity. In some embodiments, the first polypeptide further comprises a linker (e.g., a membrane proximal sequence) between the first transmembrane domain and the first intracellular domain, optionally the linker comprises the amino acid sequence of SEQ ID NO: 10, or a variant having at least about 80% (such as at least about any one of 80%, 85%, 90%, 95%, 96%, 97%, 98%, or 99%) sequence identity. In some embodiments, the second polypeptide further comprises a linker (e.g., a membrane proximal sequence) between the second transmembrane domain and the second intracellular domain, optionally the linker comprises the amino acid sequence of SEQ ID NO: 9, or a variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising the amino acid sequence of SEQ ID NO: 3, or a functional variant having at least about 80% (such as at least about any one of 80%, 85%, 90%, 95%, 96%, 97%, 98%, or 99%) sequence identity, ii) a first transmembrane domain comprising the amino acid sequence of SEQ ID NO: 7, or a variant having at least about 80% (such as at least about any one of 80%, 85%, 90%, 95%, 96%, 97%, 98%, or 99%) sequence identity, and iii) a first intracellular domain comprising the amino acid sequence of SEQ ID NO: 15, or a functional variant having at least about 80% (such as at least about any one of 80%, 85%, 90%, 95%, 96%, 97%, 98%, or 99%) sequence identity; and b) a second polypeptide comprising i) a second extracellular domain comprising the amino acid sequence of SEQ ID NO: 4, or a functional variant having at least about 80% (such as at least about any one of 80%, 85%, 90%, 95%, 96%, 97%, 98%, or 99%) sequence identity, ii) a second transmembrane domain comprising the amino acid sequence of SEQ ID NO: 8, or a variant having at least about 80% (such as at least about any one of 80%, 85%, 90%, 95%, 96%, 97%, 98%, or 99%) sequence identity, and iii) a second intracellular domain comprising the amino acid sequence of SEQ ID NO: 16, or a functional variant having at least about 80% (such as at least about any one of 80%, 85%, 90%, 95%, 96%, 97%, 98%, or 99%) sequence identity. In some embodiments, the first polypeptide further comprises a linker (e.g., a membrane proximal sequence) between the first extracellular domain and the first transmembrane domain, optionally the linker comprises the amino acid sequence of SEQ ID NO: 11, or a variant having at least about 80% (such as at least about any one of 80%, 85%, 90%, 95%, 96%, 97%, 98%, or 99%) sequence identity. In some embodiments, the second polypeptide further comprises a linker (e.g., a membrane proximal sequence) between the second extracellular domain and the second transmembrane domain, optionally the linker comprises the amino acid sequence of SEQ ID NO: 12, or a variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising the amino acid sequence of SEQ ID NO: 4, or a functional variant having at least about 80% (such as at least about any one of 80%, 85%, 90%, 95%, 96%, 97%, 98%, or 99%) sequence identity, ii) a first transmembrane domain comprising the amino acid sequence of SEQ ID NO: 7, or a variant having at least about 80% (such as at least about any one of 80%, 85%, 90%, 95%, 96%, 97%, 98%, or 99%) sequence identity, and iii) a first intracellular domain comprising the amino acid sequence of SEQ ID NO: 15, or a functional variant having at least about 80% (such as at least about any one of 80%, 85%, 90%, 95%, 96%, 97%, 98%, or 99%) sequence identity; and b) a second polypeptide comprising i) a second extracellular domain comprising the amino acid sequence of SEQ ID NO: 3, or a functional variant having at least about 80% (such as at least about any one of 80%, 85%, 90%, 95%, 96%, 97%, 98%, or 99%) sequence identity, ii) a second transmembrane domain comprising the amino acid sequence of SEQ ID NO: 8, or a variant having at least about 80% (such as at least about any one of 80%, 85%, 90%, 95%, 96%, 97%, 98%, or 99%) sequence identity, and iii) a second intracellular domain comprising the amino acid sequence of SEQ ID NO: 16, or a functional variant having at least about 80% (such as at least about any one of 80%, 85%, 90%, 95%, 96%, 97%, 98%, or 99%) sequence identity. In some embodiments, the first polypeptide further comprises a linker (e.g., a membrane proximal sequence) between the first extracellular domain and the first transmembrane domain, optionally the linker comprises the amino acid sequence of SEQ ID NO: 11, or a variant having at least about 80% (such as at least about any one of 80%, 85%, 90%, 95%, 96%, 97%, 98%, or 99%) sequence identity. In some embodiments, the second polypeptide further comprises a linker (e.g., a membrane proximal sequence) between the second extracellular domain and the second transmembrane domain, optionally the linker comprises the amino acid sequence of SEQ ID NO: 12, or a variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising i) a first extracellular domain comprising the amino acid sequence of SEQ ID NO: 3, or a functional variant having at least about 80% (such as at least about any one of 80%, 85%, 90%, 95%, 96%, 97%, 98%, or 99%) sequence identity, ii) a first transmembrane domain comprising the amino acid sequence of SEQ ID NO: 7, or a variant having at least about 80% (such as at least about any one of 80%, 85%, 90%, 95%, 96%, 97%, 98%, or 99%) sequence identity, and iii) a first intracellular domain comprising the amino acid sequence of SEQ ID NO: 17, or a variant having at least about 80% (such as at least about any one of 80%, 85%, 90%, 95%, 96%, 97%, 98%, or 99%) sequence identity; and b) a second polypeptide comprising i) a second extracellular domain comprising the amino acid sequence of SEQ ID NO: 4, or a functional variant having at least about 80% (such as at least about any one of 80%, 85%, 90%, 95%, 96%, 97%, 98%, or 99%) sequence identity, ii) a second transmembrane domain comprising the amino acid sequence of SEQ ID NO: 8, or a variant having at least about 80% (such as at least about any one of 80%, 85%, 90%, 95%, 96%, 97%, 98%, or 99%) sequence identity, and iii) a second intracellular domain comprising the amino acid sequence of SEQ ID NO: 16, or a functional variant having at least about 80% (such as at least about any one of 80%, 85%, 90%, 95%, 96%, 97%, 98%, or 99%) sequence identity. In some embodiments, the first polypeptide further comprises a linker (e.g., a membrane proximal sequence) between the first extracellular domain and the first transmembrane domain, optionally the linker comprises the amino acid sequence of SEQ ID NO: 11, or a variant having at least about 80% (such as at least about any one of 80%, 85%, 90%, 95%, 96%, 97%, 98%, or 99%) sequence identity. In some embodiments, the second polypeptide further comprises a linker (e.g., a membrane proximal sequence) between the second extracellular domain and the second transmembrane domain, optionally the linker comprises the amino acid sequence of SEQ ID NO: 12, or a variant having at least about 80% (such as at least about any one of 80%, 85%, 90%, 95%, 96%, 97%, 98%, or 99%) sequence identity.

In some embodiments, there is provided a fusion protein comprising a) a first polypeptide comprising i) a first extracellular domain comprising the amino acid sequence of SEQ ID NO: 3, or a functional variant having at least about 80% (such as at least about any one of 80%, 85%, 90%, 95%, 96%, 97%, 98%, or 99%) sequence identity, ii) a first transmembrane domain comprising the amino acid sequence of SEQ ID NO: 7, or a variant having at least about 80% (such as at least about any one of 80%, 85%, 90%, 95%, 96%, 97%, 98%, or 99%) sequence identity, and iii) a first intracellular domain comprising the amino acid sequence of SEQ ID NO: 18, or a variant having at least about 80% (such as at least about any one of 80%, 85%, 90%, 95%, 96%, 97%, 98%, or 99%) sequence identity; and b) a second polypeptide comprising i) a second extracellular domain comprising the amino acid sequence of SEQ ID NO: 4, or a functional variant having at least about 80% (such as at least about any one of 80%, 85%, 90%, 95%, 96%, 97%, 98%, or 99%) sequence identity, ii) a second transmembrane domain comprising the amino acid sequence of SEQ ID NO: 8, or a variant having at least about 80% (such as at least about any one of 80%, 85%, 90%, 95%, 96%, 97%, 98%, or 99%) sequence identity, and iii) a second intracellular domain comprising the amino acid sequence of SEQ ID NO: 19, or a variant having at least about 80% (such as at least about any one of 80%, 85%, 90%, 95%, 96%, 97%, 98%, or 99%) sequence identity. In some embodiments, the first polypeptide further comprises a linker (e.g., a membrane proximal sequence) between the first extracellular domain and the first transmembrane domain, optionally the linker comprises the amino acid sequence of SEQ ID NO: 11, or a variant having at least about 80% (such as at least about any one of 80%, 85%, 90%, 95%, 96%, 97%, 98%, or 99%) sequence identity. In some embodiments, the second polypeptide further comprises a linker (e.g., a membrane proximal sequence) between the second extracellular domain and the second transmembrane domain, optionally the linker comprises the amino acid sequence of SEQ ID NO: 12, or a variant having at least about 80% (such as at least about any one of 80%, 85%, 90%, 95%, 96%, 97%, 98%, or 99%) sequence identity.

In some embodiments, there is provided a fusion protein comprising a) a first polypeptide comprising, from N-terminus to C-terminus, i) a first extracellular domain comprising the amino acid sequence of SEQ ID NO: 3, ii) a first transmembrane domain comprising the amino acid sequence of SEQ ID NO: 5, iii) a first linker comprising the amino acid sequence of SEQ ID NO: 9, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 4, ii) a second transmembrane domain comprising the amino acid sequence of SEQ ID NO: 6, iii) a second linker comprising the amino acid sequence of SEQ ID NO: 10, and iv) a second intracellular domain comprising SEQ ID NO: 16. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising, from N-terminus to C-terminus, i) a first extracellular domain comprising the amino acid sequence of SEQ ID NO: 4, ii) a first transmembrane domain comprising the amino acid sequence of SEQ ID NO: 6, iii) a first linker comprising the amino acid sequence of SEQ ID NO: 10, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second transmembrane domain comprising the amino acid sequence of SEQ ID NO: 5, iii) a second linker comprising the amino acid sequence of SEQ ID NO: 9, and iv) a second intracellular domain comprising SEQ ID NO: 16. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising, from N-terminus to C-terminus, i) a first extracellular domain comprising the amino acid sequence of SEQ ID NO: 3, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 4, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising, from N-terminus to C-terminus, i) a first extracellular domain comprising the amino acid sequence of SEQ ID NO: 4, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising, from N-terminus to C-terminus, i) a first extracellular domain comprising the amino acid sequence of SEQ ID NO: 3, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 17; and b) a second polypeptide comprising, from N-terminus to C-terminus, i) a second extracellular domain comprising the amino acid sequence of SEQ ID NO: 4, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16.

In some embodiments, there is provided a fusion protein comprising a) a first polypeptide comprising, from N-terminus to C-terminus, i) a first extracellular domain comprising the amino acid sequence of SEQ ID NO: 3, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 18; and b) a second polypeptide comprising, from N-terminus to C-terminus, i) a second extracellular domain comprising the amino acid sequence of SEQ ID NO: 4, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 19.

In some embodiments, there is provided a fusion protein comprising a) a first polypeptide comprising the amino acid sequence of SEQ ID NO: 49, or a functional variant having at least about 80% (such as at least about any one of 80%, 85%, 90%, 95%, 96%, 97%, 98%, or 99%) sequence identity, and b) a second polypeptide comprising the amino acid sequence of SEQ ID NO: 50, or a functional variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising the amino acid sequence of SEQ ID NO: 50, or a functional variant having at least about 80% (such as at least about any one of 80%, 85%, 90%, 95%, 96%, 97%, 98%, or 99%) sequence identity, and b) a second polypeptide comprising the amino acid sequence of SEQ ID NO: 49, or a functional variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising the amino acid sequence of SEQ ID NO: 51, or a functional variant having at least about 80% (such as at least about any one of 80%, 85%, 90%, 95%, 96%, 97%, 98%, or 99%) sequence identity, and b) a second polypeptide comprising the amino acid sequence of SEQ ID NO: 52, or a functional variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising the amino acid sequence of SEQ ID NO: 53, or a functional variant having at least about 80% (such as at least about any one of 80%, 85%, 90%, 95%, 96%, 97%, 98%, or 99%) sequence identity, and b) a second polypeptide comprising the amino acid sequence of SEQ ID NO: 54, or a functional variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising the amino acid sequence of SEQ ID NO: 55, or a functional variant having at least about 80% (such as at least about any one of 80%, 85%, 90%, 95%, 96%, 97%, 98%, or 99%) sequence identity, and b) a second polypeptide comprising the amino acid sequence of SEQ ID NO: 56, or a functional variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a) a first polypeptide comprising the amino acid sequence of SEQ ID NO: 56, or a functional variant having at least about 80% (such as at least about any one of 80%, 85%, 90%, 95%, 96%, 97%, 98%, or 99%) sequence identity, and b) a second polypeptide comprising the amino acid sequence of SEQ ID NO: 55, or a functional variant having at least about 80% (such as at least about any one of 80%, 85%, 90%, 95%, 96%, 97%, 98%, or 99%) sequence identity. In some embodiments, a signal mediated via IL-23 receptor complex intracellular domain is transmitted from extracellular domain to intracellular domain of the fusion protein upon binding of TGFβ.

In some embodiments, there is provided a fusion protein comprising an amino acid sequence set forth in any of SEQ ID NOs: 20-27 (e.g., SEQ ID NOs: 20-25), or a functional variant having at least about 80% (such as at least about any one of 80%, 85%, 90%, 95%, 96%, 97%, 98%, or 99%) sequence identity.

In some embodiments, the first polypeptide and/or the second polypeptide further comprises a signal peptide at the N-terminus of the polypeptide. In some embodiments, the signal peptide is a signal peptide from TGFβR1 or TGFβR2, optionally the signal peptide and the extracellular domain in the polypeptide are derived from the same molecule. In some embodiments, the signal peptide has the amino acid sequence of SEQ ID NO: 1 or 2. In some embodiments, the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted.

In some embodiments, the first polypeptide and the second polypeptide are in a single polypeptide, and wherein the first polypeptide and the second polypeptide are separated by a multicistronic element. In some embodiments, the multicistronic element comprises a 2A self-cleaving peptide selected from the group consisting of T2A, P2A, E2A, or F2A. In some embodiments, the first polypeptide is N-terminal to the second polypeptide. In some embodiments, the first polypeptide is C-terminal to the second polypeptide.

In some embodiments, there is provided a fusion protein comprising a single polypeptide comprises, from N-terminus to C-terminus: the first extracellular domain comprising an extracellular domain of TGFβR1, the first transmembrane domain comprising a transmembrane domain of TGFβR1, the first intracellular domain comprising an intracellular domain of IL-23R, a 2A self-cleaving peptide, the second extracellular domain comprising an extracellular domain of TGFβR2, the second transmembrane domain comprising a transmembrane domain of TGFβR2, and the second intracellular domain comprising an intracellular domain of IL-12Rβ1. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a single polypeptide comprises, from N-terminus to C-terminus: the first extracellular domain comprising an extracellular domain of TGFβR2, the first transmembrane domain comprising a transmembrane domain of TGFβR2, the first intracellular domain comprising an intracellular domain of IL-23R, a 2A self-cleaving peptide, the second extracellular domain comprising an extracellular domain of TGFβR1, the second transmembrane domain comprising a transmembrane domain of TGFβR1, and the second intracellular domain comprising an intracellular domain of IL-12Rβ1. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a single polypeptide comprises, from N-terminus to C-terminus: the first extracellular domain comprising an extracellular domain of TGFβR1, the first transmembrane domain comprising a transmembrane domain of TGFβR1, the first intracellular domain comprising an intracellular domain of IL-12Rβ1, a 2A self-cleaving peptide, the second extracellular domain comprising an extracellular domain of TGFβR2, the second transmembrane domain comprising a transmembrane domain of TGFβR2, and the second intracellular domain comprising an intracellular domain of IL-23R. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a single polypeptide comprises, from N-terminus to C-terminus: the first extracellular domain comprising an extracellular domain of TGFβR1, the first transmembrane domain comprising a transmembrane domain of IL-23R, the first intracellular domain comprising an intracellular domain of IL-23R, a 2A self-cleaving peptide, the second extracellular domain comprising an extracellular domain of TGFβR2, the second transmembrane domain comprising a transmembrane domain of IL-12Rβ1, and the second intracellular domain comprising an intracellular domain of IL-12Rβ1. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a single polypeptide comprises, from N-terminus to C-terminus: the first extracellular domain comprising an extracellular domain of TGFβR2, the first transmembrane domain comprising a transmembrane domain of IL-23R, the first intracellular domain comprising an intracellular domain of IL-23R, a 2A self-cleaving peptide, the second extracellular domain comprising an extracellular domain of TGFβR1, the second transmembrane domain comprising a transmembrane domain of IL-12Rβ1, and the second intracellular domain comprising an intracellular domain of IL-12Rβ1. In some embodiments, a signal mediated via IL-23 receptor complex intracellular domain is transmitted from extracellular domain to intracellular domain of the fusion protein upon binding of TGFβ.

In some embodiments, there is provided a fusion protein comprising a single polypeptide comprises, from N-terminus to C-terminus: the first extracellular domain comprising an extracellular domain of TGFβR1, the first transmembrane domain comprising a transmembrane domain of IL-12Rβ1, the first intracellular domain comprising an intracellular domain of IL-12Rβ1, a 2A self-cleaving peptide, the second extracellular domain comprising an extracellular domain of TGFβR2, the second transmembrane domain comprising a transmembrane domain of IL-23R, and the second intracellular domain comprising an intracellular domain of IL-23R. In some embodiments, a signal mediated via IL-23 receptor complex intracellular domain is transmitted from extracellular domain to intracellular domain of the fusion protein upon binding of TGFβ.

In some embodiments, there is provided a fusion protein encoded by a nucleic acid comprising a nucleic acid sequence set forth in any of SEQ ID NOs: 28-35 (e.g., SEQ ID NOs: 28-33), or a variant having at least about 80% (such as at least about any one of 80%, 85%, 90%, 95%, 96%, 97%, 98%, or 99%) sequence identity.

### TGFβ Signaling, TGFβR1 and TGFβR2

TGF-β, comprising three isoforms, is a potent pleiotropic cytokine that regulates mammalian development, differentiation, and homeostasis in nearly all cell types and tissues. Each of three TGF-β isoforms, TGF-β1, TGF-β2, and TGF-β3, is initially synthesized as a 75-kDa homodimer known as pro-TGF-β. Pro-TGF-β is then cleaved in the Golgi to form the mature TGF-β homodimer. These 25-kDa homodimers interact with latency-associated proteins to form the small latent complex. In the endoplasmic reticulum, a single latent TGF-β binding protein forms a disulfide bond with the TGF-β homodimer to form the large latent complex, allowing for targeted export to the extracellular matrix. After export, the large latent complex interacts with fibronectin fibrils and heparin sulfate proteoglycans on the cell membrane. Eventually, the large latent complex localizes to fibrillin-rich microfibrils in the extracellular matrix, where it is stored until its activation. There, latent TGF-β is stored, where it remains biologically unavailable until its activation. Latent TGF-β is activated by several factors, including proteases, thrombospondin 1, reactive oxygen species, and integrins. These factors release mature TGF-β by freeing it from the microfibril-bound large latent complex. This occurs through liberation from latency-associated proteins, degradation of latent TGF-β binding protein, or modification of latent complex conformation.

Once the ligand is activated, TGF-β signaling is mediated through SMAD and non-SMAD pathways to regulate transcription, translation, microRNA biogenesis, protein synthesis, and post-translational modifications. Although the downstream effects of TGF-β are heavily context dependent, its signaling is at least partially conserved in many cell types. In the canonical pathway, the TGF-β ligand binds to the type 2 TGF-β receptor (TGFβR2) that recruits the type 1 TGF-β receptor (TGFβR1). These receptors dimerize and autophosphorylate serine/threonine residues, allowing for the phosphorylation of SMAD2 and SMAD3 by TGFβR1. The now activated SMAD proteins dissociate from the SMAD anchor for receptor activation (SARA) protein, hetero-oligomerize with SMAD4, and translocate to the nucleus, interacting with myriad transcriptional coregulators and other factors to mediate target gene expression or repression.

Multiple natural isoforms of human TGFβR1 or TGFβR2 protein have been known. Domains or regions of TGFβR1 or TGFβR2 described herein (such as extracellular domain, transmembrane domain, or membrane proximal region of TGFβR1 or TGFβR2) encompass corresponding domains or regions derived from natural isoforms of TGFβR1 or TGFβR2. In some embodiments, the corresponding domains or regions derived from a natural isoform of TGFβR1 or TGFβR2 has comparable intended effect as that of any of TGB23-1, TGB23-2, TGB23-3, TGB23-4, TGB23-5 and TGB23-6. For exemplary TGFβR1 natural isoform sequences, see NCBI accession number P36897.1 (SEQ ID NO: 66), CAF02096.2 (SEQ ID NO: 67), CAF02097.1 (SEQ ID NO: 68) and AAH71181.1 (SEQ ID NO: 69). For exemplary TGFβR2 natural isoform sequences, see NCBI accession number P37173.2 (SEQ ID NO: 70), Q62312.1 (SEQ ID NO: 71), the amino acid sequence of SEQ ID NO: 72, and ABK42378.1 (SEQ ID NO: 88).

In some embodiments, TGFβR1 described herein comprises the amino acid sequence set forth in any of the SEQ ID NOs: 66-69, or a variant (e.g., a functional variant) having at least about 80% (such as at least about any one of 80%, 85%, 90%, 95%, 96%, 97%, 98%, or 99%) sequence identity.

In some embodiments, TGFβR2 described herein comprises the amino acid sequence set forth in any of the SEQ ID NOs: 70-72 and 88, or a variant (e.g., a functional variant) having at least about 80% (such as at least about any one of 80%, 85%, 90%, 95%, 96%, 97%, 98%, or 99%) sequence identity.

Encompassed herein also include variants of TGFβR1 or TGFβR2 or a portion (e.g., a domain, e.g., an extracellular domain or a transmembrane domain) thereof. In some embodiments, TGFβR1 and/or TGFβR2 has any one or more (e.g., 2, 3, 4, 5 or more) modifications (e.g., a substitution, truncation, deletion, addition, such as any of the modification described herein) in the extracellular domain and/or the transmembrane domain. In some embodiments, the variant or modified TGFβR1 and/or TGFβR2 binds to TGFβ in an equivalent or increased manner than the natural isoform of TGFβR1 and/or TGFβR2 (e.g., canonical sequence of TGFβR1 and/or TGFβR2, e.g., SEQ ID NO: 66 or 70). In some embodiments, the variant or modified TGFβR1 and/or TGFβR2 transmits TGFβ signaling in an equivalent or increased manner than the natural isoform of TGFβR1 and/or TGFβR2 (e.g., canonical sequence of TGFβR1 and/or TGFβR2, e.g., SEQ ID NO: 66 or 70).

In some embodiments, TGFβR1 has a deletion of amino acids at position 24-26 according to the numbering set forth in SEQ ID NO: 66. In some embodiments, TGFβR1 has an insertion of Alanine at position 26 according to the numbering set forth in SEQ ID NO: 66. In some embodiments, TGFβR1 has an I139V substitution according to the numbering set forth in SEQ ID NO: 66. In some embodiments, TGFβR1 has a V153I substitution according to the numbering set forth in SEQ ID NO: 66.

In some embodiments, TGFβR2 has a M36V substitution according to the numbering set forth in SEQ ID NO: 70. In some embodiments, TGFβR2 has a C61R substitution according to the numbering set forth in SEQ ID NO: 70. In some embodiments, TGFβR2 has an I73V substitution according to the numbering set forth in SEQ ID NO: 70. In some embodiments, TGFβR2 has a R190H substitution according to the numbering set forth in SEQ ID NO: 70. In some embodiments, TGFβR2 has a V191I substitution according to the numbering set forth in SEQ ID NO: 70.

### IL-23 Signaling, IL23R and IL-12Rβ1

Interleukin 23 (IL-23) is a pro-inflammatory cytokine implicated in the resolution of infections caused by particular extracellular pathogens. In addition, several reports have established a solid connection between excessive IL-23 production and the development of inflammatory diseases in murine models of experimental autoimmune encephalomyelitis (EAE), psoriasis, and inflammatory bowel disease. See e.g., Cua et al. Nature. 2003; 421: 744-748. Furthermore, increased levels of IL-23 have been found in biopsies from patients with Crohn disease, ulcerative colitis, and psoriasis.

The role of IL-23 in tumor biology remains perplexing. See e.g., Yan et al., Cold Spring Harb Perspect Biol. 2018 Jul. 2; 10(7):a028530. On one hand, various studies appear to suggest that IL-23 promotes tumor growth and metastasis. A seminal paper by Langowski et al. (Nature 442: 461-465, 2006) provided the first demonstration that mice deficient in IL-23p19 were resistant to DMBA/TPA-induced skin papillomas and this resistance correlated with a significant increase in CD8+ T cells infiltrating the skin and a reduction in IL-17A, matrix metallopeptidase 9 (MMP9), CD31, granulocytes (Gr-1+), and macrophages (CD11b+, F4/80+). A study by Teng et al. (Proc Natl Acad Sci 107: 8328-8333, 2010) further confirmed the resistance phenotype of these mice to DMBA/TPA-induced skin papillomas and also highlighted their resistance to MCA-induced fibrosarcomas. Furthermore, this study also showed that, not only did IL-23 suppress the antitumor function of T cells as first uncovered by Langowski et al, it also suppressed the antimetastatic function of NK cells (Teng et al. 2010). In two other de novo mouse models of colon carcinogenesis (CPC-APC, ApcMin/+), IL-23 and IL-17A both had tumor-promoting effects as loss of IL-23/IL-23R/IL-17R or IL-23R/IL-17A blockade resulted in reduced tumor load (Wu et al. Nat Med 15: 1016-1022, 2009; Grivennikov et al., Nature 491: 254-258, 2012). On the other hand, there are also studies suggesting that IL-23 can have tumor suppressing effects when it is overexpressed in different tumor cell lines and implanted into mice. (e.g., Ngiow et al., Trends Immunol 34: 548-555, 2013).

IL-23 consists of a subunit called p40 (interleukin 12 subunit beta, Uniprot ID: P29460) and a subunit called p19 (interleukin 23 subunit alpha, Uniprot ID Q9NPF7) (Oppmann et al. Immunity 13:715-725 (2000)). The subunit p40 is a common subunit shared with interleukin 12 (i.e., IL-12). It has been suggested that IL-12 and IL-23 play conflicting roles in cancer. See e.g., Yan et al., Cold Spring Harb Perspect Biol. 2018 Jul. 2; 10(7):a028530. Although the antitumor and antimetastatic activities of IL-12 are thought to be mediated by STAT4 activation of IFN-γ, the mechanism of action of IL-23 is not fully elucidated.

IL-23 binds to and signals through its heterodimeric receptor complex (i.e., IL-23 receptor) composed of IL-12Rβ1 and IL-23R (Uniprot ID: Q5VWK5) subunits. Whereas IL-12Rβ1 is also part of the IL-12 receptor (which is a heterodimeric receptor formed by IL-12Rβ1 (Uniprot ID: P42701) and IL-12Rβ2 (Uniprot ID: Q99665)), IL-23R is unique to the IL-23 receptor complex. IL-23R pairs with IL-12Rβ1 to confer IL-23 responsiveness on cells expressing both subunits. Either inhibition of IL-12Rβ1 or IL-23R will result in blockage of IL-23 responses of cells expressing the two receptor chains (Parham, C. et al. J Immunol 168:5699-5708 (2002)).

IL-23R associates with JAK2 and in a ligand-dependent manner with STAT3. IL-12Rβ1 interacts directly with Tyk2. IL-23 binding to IL-23 receptor causes phosphorylation and activation of JAK-STAT signaling molecules: JAK2, Tyk2,

and STAT1, STAT3, STAT4, STAT5. The most significant STAT induced by IL-23 is STAT3. IL-23 induced activation of STAT3 leads to direct binding of phosphorylated STAT3 to IL-17 and IL-17F promoters. STAT3 also up-regulates the expression of ROR-gamma, a Th17 specific transcriptional regulator that is critical for the expression of two members of Interleukin-17 family, IL-17 and IL-17F. IL-23 induced JAK2 activation triggers PI3K cat class IA/AKT(PKB) and NF-kB p50/p65 pathways which are required for IL-17 production. AKT(PKB) can activate both NF-kB p50/p65 (IKK-alpha/IKK (cat)/I-kB pathway) and STAT3 (via an undetermined mechanism).

Multiple natural isoforms of human IL-23R or IL-12Rβ1 protein have been known. Domains or regions of IL-23R or IL-12Rβ1 described herein (such as transmembrane domain, membrane proximal region, or intracellular domain of IL-23R or IL-12Rβ1) encompass corresponding domains or regions derived from various natural isoforms of IL-23R or IL-12Rβ1. In some embodiments, the corresponding domains or regions derived from a natural isoform of IL-23R or IL-12Rβ1 has comparable intended effect as that of any of TGB23-1, TGB23-2, TGB23-3, TGB23-4, TGB23-5 and TGB23-6. For exemplary IL-23R natural isoform sequences, see NCBI accession number AAM44229.1 (SEQ ID NO: 73), AAY18347.1 (SEQ ID NO: 74), AAY18349.1 (SEQ ID NO: 75) and amino acid sequence set forth in SEQ ID NO: 76 and 77. For exemplary IL-12Rβ1 natural isoform sequences, see NCBI accession number CAC10446.1 (SEQ ID NO: 78) and UniProt P42701-2 (SEQ ID NO: 79).

In some embodiments, IL-23R described herein comprises the amino acid sequence set forth in any of the SEQ ID NOs: 73-77, or a variant (e.g., a functional variant) having at least about 80% (such as at least about any one of 80%, 85%, 90%, 95%, 96%, 97%, 98%, or 99%) sequence identity.

In some embodiments, IL-12Rβ1 described herein comprises the amino acid sequence set forth in any of the SEQ ID NOs: 78-79, or a variant (e.g., a functional variant) having at least about 80% (such as at least about any one of 80%, 85%, 90%, 95%, 96%, 97%, 98%, or 99%) sequence identity.

Encompassed herein also include variants of IL-23R or IL-12Rβ1 or a portion (e.g., a domain, e.g., a transmembrane domain or intracellular domain) thereof. In some embodiments, Il-23R and/or IL-12Rβ1 has any one or more (e.g., 2, 3, 4, 5 or more) modifications (e.g., a substitution, truncation, deletion, addition, such as any of the modification described herein) in the transmembrane domain and/or the intracellular domain. In some embodiments, the variant or modified IL-23R and/or IL-12Rβ1 transmits signaling via IL-23R/IL-12Rβ1 in an equivalent or increased manner than the natural isoform of IL-23R and/or IL-12Rβ1 (e.g., canonical sequence of IL-23R and/or IL-12Rβ1, e.g., SEQ ID NO: 73 or 78).

In some embodiments, IL-23R has any one or more substitutions selected from the group consisting of R381Q, V475A, N481D, and S581R substitution according to the numbering set forth in SEQ ID NO: 73.

In some embodiments, the nucleotide encoding IL-23R has a modification in the 3′-untranslated region of the IL-23R gene as described in Zwiers et al. See e.g., rs10889677 variant discussed in Zwiers et al., The Journal of Immunology. 2012; 188: 1573-1577.

### Transmembrane Domain

In some embodiments, the first transmembrane domain and the second transmembrane domain each comprises a transmembrane domain of one of TGFβR1, TGFβR2, IL-12Rβ1 and IL-23R. In some embodiments, wherein the first transmembrane domain and the second transmembrane each comprises a transmembrane domain of one of IL-12Rβ1 or IL-23R. In some embodiments, the transmembrane domain and the extracellular domain in the first and/or second polypeptide are derived from the same molecule. In some embodiments, the transmembrane domain and the intracellular domain in the first and/or second polypeptide are derived from the same molecule.

In some embodiments, the transmembrane domain of TGFβR1 comprises the amino acid sequence of SEQ ID NO: 5, or a functional variant having at least about 80% (such as at least about any one of 80%, 85%, 90%, 95%, 96%, 97%, 98%, or 99%) sequence identity. In some embodiments, the transmembrane domain of TGFβR2 comprises the amino acid sequence of SEQ ID NO: 6, or a functional variant having at least about 80% (such as at least about any one of 80%, 85%, 90%, 95%, 96%, 97%, 98%, or 99%) sequence identity. In some embodiments, the transmembrane domain of IL-23R comprises the amino acid sequence of SEQ ID NO: 7, or a functional variant having at least about 80% (such as at least about any one of 80%, 85%, 90%, 95%, 96%, 97%, 98%, or 99%) sequence identity. In some embodiments, the transmembrane domain of IL-12Rβ1 comprises the amino acid sequence of SEQ ID NO: 8, or a functional variant having at least about 80% (such as at least about any one of 80%, 85%, 90%, 95%, 96%, 97%, 98%, or 99%) sequence identity.

### Linker (e.g., Membrane Proximal Sequence)

In some embodiments, the first polypeptide or the second polypeptide further comprises a linker (e.g., membrane proximal sequence) between two domains, wherein one of the domains is the transmembrane domain, and the other domain is the extracellular domain or the intracellular domain. Without being bound to the theory, the linker (e.g., the membrane proximal sequence) may serve as a transition between two function domains to provide flexibility and/or promote lifespan of the fusion protein or signal transduction. In some embodiments, the linker (e.g., the membrane proximal sequence) is between the extracellular domain and the transmembrane domain. In some embodiments, the linker (e.g., the membrane proximal sequence) is between the transmembrane domain and the intracellular domain.

### Exemplary Membrane Proximal Sequence

In some embodiments, the linker is a membrane proximal sequence derived from a membrane proximal region from a transmembrane molecule. In some embodiments, the membrane proximal sequence and the transmembrane domain are derived from the same molecule.

In some embodiments, the membrane proximal sequence that is between the extracellular domain and the transmembrane domain is a truncated C-terminal sequence of the extracellular domain derived from the same molecule wherein the transmembrane domain is from. In some embodiments, the membrane proximal sequence that is between the transmembrane domain and the intracellular domain is a truncated N-terminal sequence of the intracellular domain derived from the same molecule wherein the transmembrane domain is from. In some embodiments, the membrane proximal sequence has a length of about 2 amino acids to about 50 amino acids (such as about 3 amino acids to about 40 amino acids. In some embodiments, the membrane proximal sequence comprises the amino acid sequence set forth in any of SEQ ID NOs: 9-12. “A sequence of IL-23R extracellular domain near the membrane”, “a sequence of IL-12Rβ1 extracellular domain near the membrane”, “a sequence of TGFβR1 intracellular domain near the membrane” and “a sequence of TGFβR2 intracellular domain near the membrane” refer to the different membrane proximal sequences used in the exemplary embodiments.

### Other Exemplary Linkers

Exemplary linkers also include any of the peptide or non-peptide linkers described below. In some embodiment, a linker (such as peptide linker) comprises flexible residues (such as glycine and serine) so that the adjacent domains are free to move relative to each other.

Exemplary linkers also include any of the peptide or non-peptide linkers described below. In some embodiment, a linker (such as peptide linker) comprises flexible residues (such as glycine and serine) so that the adjacent domains are free to move relative to each other.

### Peptide Linkers

In some embodiments, the linker is a peptide linker. In some embodiments, the peptide linker has a length of about one to about fifty, about two to about fourth, about three to about thirty, or about four to about twenty amino acids. In some embodiments, the linker is a GS linker.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ (SEQ ID NO: 61), glycine-serine polymers (including, for example, (GS)ₙ (SEQ ID NO: 62), (GSGGS)ₙ (SEQ ID NO: 63), (GGGGS)ₙ (SEQ ID NO: 64), and (GGGS)ₙ (SEQ ID NO: 65), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of a fusion protein can include linkers that are all or partially flexible, such that the linker can include a flexible linker portion as well as one or more portions that confer less flexible structure to provide a desired fusion protein structure. In some embodiments, the linker is a GS linker.

In some embodiments, the peptide linker comprises the amino acid sequence set forth in SEQ ID NO: 57.

In some embodiments, the peptide linker comprises the hinge region of an IgG, such as the hinge region of human IgG1. In some embodiments, the peptide linker comprises the hinge region of an IgG, such as the hinge region of human IgG1. In some embodiments, the peptide linker comprises a modified sequence derived from the hinge region of an IgG, such as the hinge region of human IgG1.

### III. Nucleic Acids and Vectors

The present application also provides nucleic acids that encode any of the fusion proteins described herein, or a portion thereof.

In some embodiments, the nucleic acid comprises a nucleic acid sequence set forth in any of SEQ ID NOs: 28-35, or a variant having at least about 80% (such as at least about any one of 80%, 85%, 90%, 95%, 96%, 97%, 98%, or 99%) sequence identity. In some embodiments, the nucleic acid comprises a nucleic acid sequence set forth in any of SEQ ID NOs: 28-33, or a variant having at least about 80% (such as at least about any one of 80%, 85%, 90%, 95%, 96%, 97%, 98%, or 99%) sequence identity.

In some embodiments, the nucleic acid further comprises a second nucleic acid sequence encoding a functional exogenous receptor (such as any of the functional exogenous receptors described in the “functional exogenous receptors” section). In some embodiments, the functional exogenous receptor comprises an extracellular ligand-binding domain and optionally an intracellular signaling domain. In some embodiments, the functional exogenous receptor is selected from the group consisting of: an engineered T cell receptor (TCR), a chimeric antigen receptor (CAR), a T cell antigen coupler (TAC) or a portion thereof. In some embodiments, the functional exogenous receptor specifically recognizes a tumor antigen. In some embodiments, the functional exogenous receptor comprises a CAR. In some embodiments, the CAR comprises the amino acid sequence of SEQ ID NO: 36, or a variant having at least about 80% (such as at least about any one of 80%, 85%, 90%, 95%, 96%, 97%, 98%, or 99%) sequence identity. In some embodiments, the CAR comprises the amino acid sequence of SEQ ID NO: 89, or a variant having at least about 80% (such as at least about any one of 80%, 85%, 90%, 95%, 96%, 97%, 98%, or 99%) sequence identity.

In some embodiments, the nucleic acid encodes a polypeptide comprising the amino acid sequence of SEQ ID NO: 37, or a variant having at least about 80% (such as at least about any one of 80%, 85%, 90%, 95%, 96%, 97%, 98%, or 99%) sequence identity.

In some embodiments, the nucleic acid comprises a nucleic acid sequence set forth in SEQ ID NO: 38, or a variant having at least about 80% (such as at least about any one of 80%, 85%, 90%, 95%, 96%, 97%, 98%, or 99%) sequence identity.

In some embodiments, the nucleic acid encodes a polypeptide comprising the amino acid sequence of SEQ ID NO: 90, or a variant having at least about 80% (such as at least about any one of 80%, 85%, 90%, 95%, 96%, 97%, 98%, or 99%) sequence identity.

In some embodiments, the nucleic acid sequence encoding the functional exogenous receptor is upstream to at least one of the one or more nucleic acid sequences encoding the fusion protein (“fusion protein nucleic acid sequence”), and optionally wherein functional exogenous receptor nucleic acid sequence and the fusion protein nucleic acid sequence are separated by a third nucleic acid sequence encoding a second multicistronic element.

In some embodiments, the nucleic acid sequence encoding the functional exogenous receptor is downstream to at least one of the one or more nucleic acid sequences encoding the fusion protein (“fusion protein nucleic acid sequence”), and optionally wherein functional exogenous receptor nucleic acid sequence and the fusion protein nucleic acid sequence are separated by a third nucleic acid sequence encoding a second multicistronic element.

In some embodiments, the second multicistronic element comprises a 2A self-cleaving peptide selected from the group consisting of T2A, P2A, E2A, or F2A.

### Vectors

The present application provides vectors for cloning and expressing any one of fusion proteins and/or functional exogenous receptor (e.g., engineered TCR or CAR) described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, herpes simplex viral vector, and derivatives thereof. In some embodiments, the vector is a lentiviral vector. See e.g., Example 2. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the fusion protein coding sequence and/or self-inactivating lentiviral vectors carrying exogenous receptor (e.g., engineered TCR, CAR) can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly(lactic-co-glycolic acid) (PLGA) and poly lactic acid (PLA), poly(ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the fusion protein-encoding nucleic acid and/or exogenous receptor (e.g., engineered TCR, CAR)-encoding nucleic acid to the engineered immune effector cells (e.g., T cell). See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids encoding the fusion protein and/or exogenous receptor (e.g., engineered TCR, CAR) described herein is introduced to the engineered immune effector cells (e.g., T cell) by a physical method, including, but not limited to electroporation, sonoporation, photoporation, magnetofection, hydroporation.

In some embodiments, the vector (e.g., viral vector such as lentiviral vector) comprises any one of the nucleic acids encoding the fusion protein and/or the exogenous receptor (e.g., engineered TCR, CAR) described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters.

### Promoters

In some embodiments, the nucleic acid encoding the fusion protein and/or the exogenous receptor (e.g., engineered TCR, CAR) described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken R-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. For example, Michael C. Milone et al. compared the efficiencies of CMV, hEF1α, UbiC and PGK to drive CAR expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the fusion protein and/or the exogenous receptor (e.g., engineered TCR, CAR) described herein is operably linked to a hEF1α promoter or a PGK promoter.

In some embodiments, the promoter is selected from the group consisting of an EF-1 promoter, a CMV IE gene promoter, an EF-1a promoter, an ubiquitin C promoter, a phosphoglycerate kinase (PGK) promoter, a Rous Sarcoma Virus (RSV) promoter, an Simian Virus 40 (SV40) promoter a cytomegalovirus immediate early gene promoter (CMV), an elongation factor 1 alpha promoter (EF1-α), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MC1), a beta actin promoter (β-ACT), a simian virus 40 promoter (SV40), and a myeloproliferative sarcoma virus enhancer, negative control region deleted, d1587rev primer-binding site substituted (MND) promoter, an NFAT promoter, a TETON® promoter, and an NFκB promoter.

In some embodiments, the nucleic acid encoding the fusion protein and/or the exogenous receptor (e.g., engineered TCR, CAR) described herein is operably linked to an inducible promoter. Inducible promoters belong to the category of regulated promoters. The inducible promoter can be induced by one or more conditions, such as a physical condition, microenvironment of the engineered immune effector cell (e.g., T cell), or the physiological state of the engineered immune effector cell, an inducer (i.e., an inducing agent), or a combination thereof. In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell. In some embodiments, the inducible promoter can be an NFAT promoter, a TETON® promoter, or an NFκB promoter.

In some embodiments, the vector also contains a selectable marker gene or a reporter gene to select cells expressing the fusion protein and/or the exogenous receptor (e.g., engineered TCR, CAR) described herein from the population of host cells transfected through vectors (e.g., lentiviral vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### Multicistronic Element

In some embodiments, the vector comprises more than one nucleic acids encoding the fusion protein and/or the exogenous receptor (e.g., engineered TCR, CAR) described herein. In some embodiments, the vector (e.g., viral vector such as a lentiviral vector) comprises a first nucleic acid encoding a fusion protein and a second nucleic acid encoding a functional exogenous receptor comprising an extracellular ligand-binding domain and an optional intracellular signaling domain (e.g., engineered TCR, CAR), wherein the first nucleic acid is operably linked to the second nucleic acid via a third nucleic acid encoding a multicistronic element. In some embodiments, the third nucleic acid encoding a multicistronic element is an internal ribosome entry site (IRES). IRES is an RNA element that allows for translation initiation in a cap-independent manner. In some embodiments, the multicistronic element comprises (e.g., is) a self-cleaving 2A peptide, such as P2A, T2A, E2A, F2A, BmCPV 2A, BmIFV 2A. In some embodiments, the multicistronic element is a P2A peptide comprising the amino acid sequence of SEQ ID NO: 48 or 60. In some embodiments, the multicistronic element comprises a peptide linker as described in the above “peptide linkers” section under “II. Fusion proteins”, such as a flexible linker. In some embodiments, the flexible linking sequence is selected from the group consisting of (GS)ₙ, (GSGGS)ₙ (GGGS)ₙ, and (GGGGS)ₙ, where n is an integer of at least one). In some embodiments, the third nucleic acid encodes a selectable marker, such as LNGFR. In some embodiments, the third nucleic acid encoding a multicistronic element comprising one or more types of the linking sequences described herein, such as a self-cleaving 2A peptide (e.g., P2A) followed by a Gly-Ser flexible linker (e.g., (GGGS)₃), or a self-cleaving 2A peptide (e.g., P2A) followed by a selectable marker (e.g., LNGFR).

Thus in some embodiments, there is provided a vector (e.g., viral vector such as lentiviral vector) comprising a first nucleic acid encoding a fusion protein (such as any of the fusion proteins described herein). In some embodiments, the fusion protein comprises a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1.

In some embodiments, the vector (e.g., viral vector such as lentiviral vector) further comprises a second nucleic acid encoding a functional exogenous receptor comprising an extracellular ligand-binding domain and an optional intracellular signaling domain (e.g., engineered TCR, CAR). In some embodiments, the first nucleic acid and the second nucleic acid are operably linked to the same promoter. In some embodiments, the first nucleic acid and the second nucleic acid are operably linked to different promoters.

In some embodiments, there is provided a vector (e.g., viral vector such as a lentiviral vector) comprising a first nucleic acid encoding a fusion protein (such as any of the fusion proteins described herein) and a second nucleic acid encoding a functional exogenous receptor comprising an extracellular ligand-binding domain and an intracellular signaling domain (e.g., engineered TCR, CAR), wherein the first nucleic acid and the second nucleic acid are operably linked to different promoters (e.g., EF1-α and a promoter different from EF1-α). In some embodiments, the first nucleic acid is upstream of the second nucleic acid. In some embodiments, the first nucleic acid is downstream of the second nucleic acid.

In some embodiments, there is provided a vector (e.g., a viral vector, such as a lentiviral vector) from upstream to downstream: a promoter (e.g., EF1-α), a first nucleic acid encoding a functional exogenous receptor comprising an extracellular ligand-binding domain and an intracellular signaling domain (e.g., engineered TCR, CAR), optionally a second promoter, and a second nucleic acid encoding the fusion protein. In some embodiments, the fusion protein comprises a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1.

### Functional Exogenous Receptors

Exemplary functional exogenous receptors described herein can comprise an extracellular ligand-binding domain and optionally an intracellular signaling domain. In some embodiments, the functional exogenous receptor is an engineered TCR (e.g., an engineered TCR specifically recognizing a tumor antigen). In some embodiments, the functional exogenous receptor is a non-TCR receptor. In some embodiments, the non-TCR receptor is a chimeric antigen receptor (CAR) comprising: (a) an extracellular ligand-binding domain comprising one or more (such as any one of 1, 2, 3, 4, 5, 6 or more) binding moieties specifically recognizing an antigen (e.g., a tumor antigen); (b) a transmembrane domain; and (c) an intracellular signaling domain. In some embodiments, the extracellular ligand-binding domain of the CAR comprises one or more (such as any one of 1, 2, 3, 4, 5, 6 or more) binding moieties comprising an antigen-binding fragment (hereinafter referred to as “anti-antigen CAR”), such as sdAbs (e.g., an sdAb that targets a tumor antigen). In some embodiments, the extracellular ligand-binding domain of the CAR comprises one or more binding moieties comprising at least one domain derived from a ligand or the extracellular domain of a receptor (hereinafter also referred to as “ligand/receptor CAR”), wherein the ligand or receptor is a cell surface antigen. In some embodiments, the receptor is derived from an Fc binding domain, such as an extracellular domain of an Fc receptor (e.g., FcγR). CARs comprising an extracellular ligand-binding domain comprising one or more binding moieties comprising an Fc binding domain is hereinafter also referred to as “antibody-coupled T cell receptor (ACTR)”. In some embodiments, when an Fc-containing protein is administered to or co-expressed in an ACTR-T cell, the Fc-containing protein confers binding specificity of the ACTR-expressing T cell to an antigen described herein. In some embodiments, the Fc-containing protein is an Fc-containing antibody (e.g., a full-length antibody) or an Fc-fusion protein, such as antigen-binding fragment-Fc fusion protein, Fc-receptor/ligand fusion protein, Fc-fusion protein comprising a variable region of a TCR fused to an Fc region of an immunoglobulin G (IgG) (“TCR-Fc fusion protein”). The ACTR/Fc-containing protein system is hereinafter referred to as “anti-antigen ACTR”.

### Antigens

The extracellular ligand-binding domain of the functional exogenous receptor described herein can specifically recognize any antigen on a target cell. In some embodiments, the antigen is a cell surface molecule. In some embodiments, the antigen acts as a cell surface marker on target cells associated with a special disease state. In some embodiments, the antigen is a tumor antigen. In some embodiments, the extracellular ligand-binding domain specifically recognizes a single tumor antigen. In some embodiments, the extracellular ligand-binding domain specifically recognizes one or more epitopes of a single tumor antigen. In some embodiments, the extracellular ligand-binding domain specifically recognizes two or more tumor antigens. Tumors express a number of proteins that can serve as a target antigen for an immune response, particularly T cell mediated immune responses. The antigens specifically recognized by the extracellular ligand-binding domain may be antigens on a single diseased cell or antigens that are expressed on different cells that each contribute to the disease. The antigens specifically recognized by the extracellular ligand-binding domain may be directly or indirectly involved in the diseases.

Tumor antigens are proteins that are produced by tumor cells that can elicit an immune response; particularly T cell mediated immune responses. The selection of the targeted antigen of the invention will depend on the particular type of cancer to be treated. Exemplary tumor antigens include, for example, a glioma-associated antigen, BCMA (B-cell maturation antigen), carcinoembryonic antigen (CEA), 3-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), Glypican 3 (GPC3), Claudin18.2, PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the tumor antigen is selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Claudin18.2, Glycolipid F77, PD-L1, PD-L2, and any combination thereof. In some embodiments, the antigen is GPC3. In some embodiments, the antigen is CD20.

In some embodiments, the antigen is a pathogen antigen, such as a fungal, viral, or bacterial antigen. In some embodiments, the fungal antigen is from Aspergillus or Candida. In some embodiments, the viral antigen is from Herpes simplex virus (HSV), respiratory syncytial virus (RSV), metapneumovirus (hMPV), rhinovirus, parainfluenza (PIV), Epstein-Barr virus (EBV), Cytomegalovirus (CMV), JC virus (John Cunningham virus), BK virus, HIV, Zika virus, human coronavirus, norovirus, encephalitis virus, or Ebola.

In some embodiments, the cell surface antigen is a ligand or receptor. In some embodiments, the extracellular ligand-binding domain comprises one or more binding moieties comprising at least one domain derived from a ligand or the extracellular domain of a receptor, wherein the ligand or receptor is a cell surface antigen described herein. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF, which can bind to BCMA. In some embodiments, the receptor is derived from an Fc binding domain, such as an extracellular domain of an Fc receptor. In some embodiments, the Fc receptor is an Fcγ receptor (FcγR). In some embodiments, the FcγR is selected from the group consisting of CD16A (FcγRIIIa), CD16B (FcγRIIIb), CD64A, CD64B, CD64C, CD32A, and CD32B.

### Chimeric Antigen Receptors (CARs)

In some embodiments, the functional exogenous receptor is a CAR comprising: (a) an extracellular ligand-binding domain comprising one or more (such as any one of 1, 2, 3, 4, 5, 6 or more) binding moieties specifically recognizing an antigen (such as any of the antigens described above, e.g., GPC3); (b) a transmembrane domain; and (c) an intracellular signaling domain. In some embodiments, the one or more binding moieties are antibodies or antigen-binding fragments thereof. In some embodiments, the one or more binding moieties are derived from one or more antibodies (e.g., full-length antibodies). In some embodiments, the one or more binding moieties are derived from camelid antibodies. In some embodiments, the one or more binding moieties are derived from human antibodies. In some embodiments, the one or more binding moieties are selected from the group consisting of a Camel Ig, Ig NAR, Fab fragments, Fab′ fragments, F(ab)′2 fragments, F(ab)′3 fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody). In some embodiments, the one or more binding moieties are sdAbs (e.g., an anti-GPC3 sdAb, an anti-BCMA sdAb). In some embodiments, the extracellular ligand-binding domain comprises two or more sdAbs linked together. In some embodiments, the one or more binding moieties are non-antibody binding proteins, such as polypeptide ligands or engineered proteins that bind to an antigen. In some embodiments, the one or more binding moieties comprise at least one domain derived from a ligand or the extracellular domain of a receptor, wherein the ligand or receptor is a cell surface antigen. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF, which can bind to BCMA. In some embodiments, the receptor is derived from an Fc binding domain, such as an extracellular domain of an Fc receptor. In some embodiments, the Fc receptor is an Fcγ receptor (FcγR). In some embodiments, the FcγR is selected from the group consisting of CD16A (FcγRIIIa), CD16B (FcγRIIIb), CD64A, CD64B, CD64C, CD32A, and CD32B. In some embodiments, the CAR is monovalent and monospecific. In some embodiments, the CAR is multivalent (e.g., bispecific) and monospecific. In some embodiments, the CAR is multivalent (e.g., bivalent) and multispecific (e.g., bispecific). In some embodiments, the antigen is selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Claudin18.2, Glycolipid F77, PD-L1, PD-L2, and any combination thereof. In some embodiments, the antigen is GPC3. In some embodiments, the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD37, CD45, CD64, CD80, CD86, CD134, CD137 (4-1BB), CD152, CD154, and PD-1. In some embodiments, the transmembrane domain is derived from CD8α. In some embodiments, the intracellular signaling domain comprises a primary intracellular signaling domain of an immune effector cell. In some embodiments, the primary intracellular signaling domain is derived from CD3ζ, CD3γ, CD3ε, CD3δ, FcRγ (FCER1G), FcRβ (Fc Epsilon RIb), CD5, CD22, CD79a, CD79b, CD66d, Fc gamma RIIa, DAP10, and DAP12. In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the co-stimulatory signaling domain comprises a cytoplasmic domain of CD137. In some embodiments, the CAR described herein further comprises a hinge domain located between the C-terminus of the extracellular ligand-binding domain and the N-terminus of the transmembrane domain. In some embodiments, the hinge domain is derived from CD8α. In some embodiments, the CAR further comprises a signal peptide located at the N-terminus of the polypeptide. In some embodiments, the signal peptide is derived from CD8α. In some embodiments, the CAR comprises a polypeptide comprising from N-terminus to C-terminus: a CD8α signal peptide, the extracellular ligand-binding domain (e.g., one or more sdAbs specifically recognizing a tumor antigen), a CD8α hinge domain, a CD8α transmembrane domain, a co-stimulatory signaling domain derived from CD137, and a primary intracellular signaling domain derived from CD3ζ.

In some embodiments, the CAR comprises a polypeptide comprising from N-terminus to C-terminus: a CD8α signal peptide, an extracellular ligand-binding domain comprising one or more binding moieties that targets a tumor antigen (e.g., an anti-GPC3 svFv or anti-CD20 scFv), a CD8α hinge domain, a CD8α transmembrane domain, a co-stimulatory signaling domain derived from CD137, and a primary intracellular signaling domain derived from CD3ζ. In some embodiments, the CAR comprises a polypeptide comprising from N-terminus to C-terminus: a CD8α signal peptide, an extracellular ligand-binding domain comprising an scFv that targets a tumor antigen (e.g., an anti-GPC3 scFv or anti-CD20 scFv), a CD8α hinge domain, a CD8α transmembrane domain, a co-stimulatory signaling domain derived from CD137, and a primary intracellular signaling domain derived from CD3ζ.

In some embodiments, the CAR of the present application is a “BCMA-ligand CAR”. In some embodiments, the CAR comprises a polypeptide comprising from N-terminus to C-terminus: a CD8α signal peptide, an extracellular ligand-binding domain comprising one or more binding moieties comprising at least one domain derived from APRIL or BAFF, a CD8α hinge domain, a CD8α transmembrane domain, a co-stimulatory signaling domain derived from CD137, and a primary intracellular signaling domain derived from CD3ζ. In some embodiments, the extracellular ligand-binding domain comprises an APRIL domain. In some embodiments, the extracellular ligand-binding domain comprises a BAFF domain. In some embodiments, the extracellular ligand-binding domain comprises an APRIL domain and a BAFF domain.

In some embodiments, the CAR of the present application is an ACTR. Engineered T cells bearing the ACTR can bind to an Fc-containing protein (such as a monoclonal antibody, e.g., anti-BCMA antibody) which then acts as a bridge to the tumor cells. In some embodiments, the CAR comprises a polypeptide comprising from N-terminus to C-terminus: a CD8α signal peptide, an extracellular ligand-binding domain comprising one or more binding moieties comprising an Fc binding domain (such as Fc receptor, e.g., FcγR), a CD8α hinge domain, a CD8α transmembrane domain, a co-stimulatory signaling domain derived from CD137, and a primary intracellular signaling domain derived from CD3ζ. In some embodiments, the FcγR is selected from the group consisting of CD16A (FcγRIIIa), CD16B (FcγRIIIb), CD64A, CD64B, CD64C, CD32A, and CD32B.

In some embodiments, the CAR comprises an amino acid sequence of SEQ ID NO: 36.

In some embodiments, the CAR comprises an amino acid sequence of SEQ ID NO: 89.

Any CAR known in the art or developed by the inventors, including the CARs described in PCT/CN2020/112181, PCT/CN2020/112182, PCT/CN2017/096938 and PCT/CN2016/094408 (the contents of which are incorporated herein by reference in their entirety), may be used to construct the CARs described herein. Exemplary structures of CARs are shown in FIGS. 15A-15D of PCT/CN2017/096938.

Multivalent and/or Multispecific CAR

In some embodiments, the CAR described herein is a multivalent CAR comprising: (a) an extracellular ligand-binding domain comprising two or more (such as any one of 2, 3, 4, 5, 6 or more) binding moieties specifically recognizing an antigen (e.g., any of the antigens described herein); (b) a transmembrane domain; and (c) an intracellular signaling domain. In some embodiments, one or more of the binding moieties are antigen-binding fragments. In some embodiments, one or more of the binding moieties comprise single-domain antibodies (e.g., anti-GPC3 sdAbs). In some embodiments, one or more of the binding moieties are derived from camelid antibodies. In some embodiments, one or more of the binding moieties are derived from a four-chain antibody. In some embodiments, one or more of the binding moieties are scFvs. In some embodiments, one or more of the binding moieties are derived from human antibodies. In some embodiments, one or more of the binding moieties are polypeptide ligands or other non-antibody polypeptides that specifically bind to the antigen. In some embodiments, the multivalent CAR is monospecific, i.e., the multivalent CAR targets a single antigen, and comprises two or more binding sites for the single antigen. In some embodiments, the multivalent CAR is multispecific, i.e., the multivalent CAR targets more than one antigen, and the multivalent CAR comprises two or more binding sites for at least one antigen. The binding moieties specific for the same antigen may bind to the same epitope of the antigen (i.e., “mono-epitope CAR”) or bind to different epitopes (i.e., “multi-epitope CAR” such as bi-epitope CAR or tri-epitope CAR) of the antigen. The binding sites specific for the same antigen may comprise the same or different sdAbs. In some embodiments, the antigen is selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Claudin18.2, Glycolipid F77, PD-L1, PD-L2, and any combination thereof. In some embodiments, the antigen is GPC3.

In some embodiments, the CAR described herein is a multivalent (such as bivalent, trivalent, or of higher number of valencies) CAR comprising: (a) an extracellular ligand-binding domain comprising a plurality (such as at least about any one of 2, 3, 4, 5, 6, or more) of binding moieties specifically binding to an antigen (such as a tumor antigen, e.g., GPC3, CD20); (b) a transmembrane domain; and (c) an intracellular signaling domain. In some embodiments, the CAR described herein is a multivalent (such as bivalent, trivalent, or of higher number of valencies) CAR comprising: (a) an extracellular ligand-binding domain comprising a plurality (such as at least about any one of 2, 3, 4, 5, 6, or more) of sdAbs specifically binding to an antigen (such as a tumor antigen, e.g., GPC3, CD20); (b) a transmembrane domain; and (c) an intracellular signaling domain. In some embodiments, the CAR described herein is a multivalent (such as bivalent, trivalent, or of higher number of valencies) CAR comprising: (a) an extracellular ligand-binding domain comprising a first binding moiety specifically binding to a first epitope of an antigen (such as a tumor antigen, e.g., GPC3, CD20), and a second binding moiety specifically binding to a second epitope of the antigen; (b) a transmembrane domain; and (c) an intracellular signaling domain. In some embodiments, the first epitope and the second epitope are different. In some embodiments, the first epitope and the second epitope are the same. In some embodiments, the first binding moiety is an sdAb and the second binding moiety is derived from a human antibody (e.g., a scFv). In some embodiments, the multivalent CAR specifically binds to two different epitopes on an antigen. In some embodiments, the multivalent CAR specifically binds to three or more different epitopes on an antigen. In some embodiments, the CAR described herein is a multivalent (such as bivalent, trivalent, or of higher number of valencies) CAR comprising: (a) an extracellular ligand-binding domain comprising a first sdAb specifically binding to a first epitope of an antigen (such as a tumor antigen, e.g., GPC3, CD20), and a second sdAb specifically binding to a second epitope of the antigen; (b) a transmembrane domain; and (c) an intracellular signaling domain. In some embodiments, the first epitope and the second epitope are different. In some embodiments, the first epitope and the second epitope are the same. In some embodiments, the CAR described herein is a multivalent (such as bivalent, trivalent, or of higher number of valencies) CAR comprising: (a) an extracellular ligand-binding domain comprising a first sdAb specifically binding to a first antigen (such as a tumor antigen, e.g., GPC3, CD20), and a second sdAb specifically binding to a second antigen; (b) a transmembrane domain; and (c) an intracellular signaling domain. In some embodiments, the first antigen and the second antigen are different. In some embodiments, the first antigen and the second antigen are the same. In some embodiments, the antigen is selected from the group consisting of CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Claudin18.2, Glycolipid F77, PD-L1, PD-L2, and any combination thereof. In some embodiments, the antigen is GPC3. In some embodiments, the antigen is CD20.

In some embodiments, the CAR described herein is a multivalent (such as bivalent, trivalent, or of higher number of valencies) CAR comprising: (a) an extracellular ligand-binding domain comprising a first sdAb specifically binding to a first epitope of GPC3 (“anti-GPC3 sdAb1” or “anti-GPC3 V_{H}H1”), and a second sdAb specifically binding to a second epitope of GPC3 (“anti-GPC3 sdAb2” or “anti-GPC3 V_{H}H2”); (b) a transmembrane domain; and (c) an intracellular signaling domain. In some embodiments, anti-GPC3 sdAb1 and anti-GPC3 sdAb2 are the same. In some embodiments, anti-GPC3 sdAb1 and anti-GPC3 sdAb2 are different.

### Extracellular Ligand-Binding Domain

The extracellular ligand-binding domain of the CARs described herein comprises one or more (such as any one of 1, 2, 3, 4, 5, 6 or more) binding moieties, such as sdAbs. In some embodiments, the one or more binding moieties are antibodies or antigen-binding fragments thereof. In some embodiments, the one or more binding moieties are derived from four-chain antibodies. In some embodiments, the one or more binding moieties are derived from camelid antibodies. In some embodiments, the one or more binding moieties are derived from human antibodies. In some embodiments, the one or more binding moieties are selected from the group consisting of a Camel Ig, Ig NAR, Fab fragments, Fab′ fragments, F(ab)′2 fragments, F(ab)′3 fragments, Fv, single chain Fv antibody (scFv), bis-scFv, (scFv)₂, minibody, diabody, triabody, tetrabody, disulfide stabilized Fv protein (dsFv), and single-domain antibody (sdAb, nanobody). In some embodiments, the one or more binding moieties are sdAbs (e.g., anti-BCMA sdAbs). In some embodiments, the one or more binding moieties are non-antibody binding proteins, such as polypeptide ligands or engineered proteins that bind to an antigen. In some embodiments, the one or more binding moieties comprise at least one domain derived from a ligand or the extracellular domain of a receptor, wherein the ligand or receptor is a cell surface antigen. In some embodiments, the ligand or receptor is derived from a molecule selected from the group consisting of NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL or BAFF, which can bind to BCMA. In some embodiments, the receptor is derived from an Fc binding domain, such as an extracellular domain of an Fc receptor. In some embodiments, the Fc receptor is an Fcγ receptor (FcγR). In some embodiments, the FcγR is selected from the group consisting of CD16A (FcγRIIIa), CD16B (FcγRIIIb), CD64A, CD64B, CD64C, CD32A, and CD32B. The binding moieties can be fused to each other directly via peptide bonds, or via peptide linkers.

In some embodiments, the extracellular ligand-binding domain of the CAR is a scFv that targets a tumor antigen. In some embodiments, the extracellular ligand-binding domain of the CAR is an anti-GPC3 scFv. In some embodiments, the anti-GPC3 scFv comprises a) a heavy chain variable region (V_{H}) comprising a VH-CDR1, a VH-CDR2, and a VH-CDR3, respectively comprising the amino acid sequences of a CDR1, a CDR2, and a CDR3 within a V_{H} chain region having the sequence set forth in any one of SEQ ID NOs: 80, and b) a light chain variable region (V_{L}) comprising a VL-CDR1, a VL-CDR2, and a VL-CDR3, respectively comprising the amino acid sequences of a CDR1, a CDR2, and a CDR3 within a V_{L} chain region having the sequence set forth in any one of SEQ ID NOs: 81. In some embodiments, the anti-GPC3 scFv comprises a) a heavy chain variable region (V_{H}) comprising a VH-CDR1 comprising amino acid sequence set forth in SEQ ID NO: 82, a VH-CDR2 comprising amino acid sequence set forth in SEQ ID NO: 83, and a VH-CDR3 comprising amino acid sequence set forth in SEQ ID NO: 84; and b) a light chain variable region (V_{L}) comprising a VL-CDR1 comprising amino acid sequence set forth in SEQ ID NO: 85, a VL-CDR2 comprising amino acid sequence set forth in SEQ ID NO: 86, and a VL-CDR3 comprising amino acid sequence set forth in SEQ ID NO: 87. In some embodiments, the anti-GPC3 scFv comprises a heavy chain variable region (V_{H}) comprising the amino acid sequence set forth in SEQ ID NO: 80, or a variant having at least about 80% (such as at least about any one of 80%, 85%, 90%, 95%, 96%, 97%, 98%, or 99%) sequence identity; and/or a light chain variable region (V_{L}) comprising the amino acid sequence set forth in SEQ ID NO: 81, or a variant having at least about 80% (such as at least about any one of 80%, 85%, 90%, 95%, 96%, 97%, 98%, or 99%) sequence identity.

In some embodiments, the anti-GPC3 scFv comprises the amino acid sequence of SEQ ID NO: 58, or a variant having at least about 80% (such as at least about any one of 80%, 85%, 90%, 95%, 96%, 97%, 98%, or 99%) sequence identity. In some embodiments, the anti-GPC3 scFv comprises the amino acid sequence of SEQ ID NO: 58.

Single-Domain Antibodies (sdAbs)

In some embodiments, the CAR comprises an extracellular ligand-binding domain comprising one or more sdAbs. The sdAbs may be of the same of different origins, and of the same or different sizes. Exemplary sdAbs include, but are not limited to, heavy chain variable domains from heavy-chain only antibodies (e.g., V_{H}H or V_{NAR}), binding molecules naturally devoid of light chains, single domains (such as V_{H} or V_{L}) derived from conventional 4-chain antibodies, humanized heavy-chain only antibodies, human sdAbs produced by transgenic mice or rats expressing human heavy chain segments, and engineered domains and single domain scaffolds other than those derived from antibodies. Any sdAbs known in the art or developed by the inventors, including the sdAbs described in PCT/CN2017/096938 and PCT/CN2016/094408 (the contents of which are incorporated herein by reference in their entirety), may be used to construct the CARs described herein. Exemplary structures of CARs are shown in FIGS. 15A-15D of PCT/CN2017/096938. The sdAbs may be derived from any species including, but not limited to mouse, rat, human, camel, llama, lamprey, fish, shark, goat, rabbit, and bovine. Single-domain antibodies contemplated herein also include naturally occurring sdAb molecules from species other than Camelidae and sharks.

In some embodiments, the sdAb is derived from a naturally occurring single-domain antigen-binding molecule known as heavy chain antibody devoid of light chains (also referred herein as “heavy chain only antibodies”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a V_{H}H to distinguish it from the conventional V_{H} of four chain immunoglobulins. Such a V_{H}H molecule can be derived from antibodies raised in Camelidae species, for example, camel, llama, vicuna, dromedary, alpaca and guanaco. Other species besides Camelidae may produce heavy chain molecules naturally devoid of light chain, and such V_{H}Hs are within the scope of the present application.

V_{H}H molecules from Camelids are about 10 times smaller than IgG molecules. They are single polypeptides and can be very stable, resisting extreme pH and temperature conditions. Moreover, they can be resistant to the action of proteases which is not the case for conventional 4-chain antibodies. Furthermore, in vitro expression of V_{H}H produces high yield, properly folded functional V_{H}Hs. In addition, antibodies generated in Camelids can recognize epitopes other than those recognized by antibodies generated in vitro through the use of antibody libraries or via immunization of mammals other than Camelids (see, for example, WO9749805). As such, multispecific or multivalent CARs comprising one or more V_{H}H domains may interact more efficiently with targets than multispecific or multivalent CARs comprising antigen binding fragments derived from conventional 4-chain antibodies. Since V_{H}Hs are known to bind into ‘unusual’ epitopes such as cavities or grooves, the affinity of CARs comprising such V_{H}Hs may be more suitable for therapeutic treatment than conventional multispecific polypeptides.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) V_{H} domain from a conventional 4-chain antibody by one or more of the amino acid residues that occur at the corresponding position(s) in a V_{H}H domain of a heavy chain antibody. This can be performed in a manner known per se, which will be clear to the skilled person, for example based on the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678, Davies and Riechmann FEBS Letters 339: 285-290, 1994; Davies and Riechmann Protein Engineering 9 (6): 531-537, 1996; Riechmann J. Mol. Biol. 259: 957-969, 1996; and Riechmann and Muyldermans J. Immunol. Meth. 231: 25-38, 1999).

In some embodiments, the sdAb is a human sdAb produced by transgenic mice or rats expressing human heavy chain segments. See, e.g., US20090307787A1, U.S. Pat. No. 8,754,287, US20150289489A1, US20100122358A1, and WO2004049794. In some embodiments, the sdAb is affinity matured.

In some embodiments, naturally occurring V_{H}H domains against a particular antigen or target, can be obtained from (naïve or immune) libraries of Camelid V_{H}H sequences. Such methods may or may not involve screening such a library using said antigen or target, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from (naïve or immune) V_{H}H libraries may be used, such as V_{H}H libraries obtained from (naïve or immune) V_{H}H libraries by techniques such as random mutagenesis and/or CDR shuffling, as for example described in WO 00/43507.

In some embodiments, the sdAbs are generated from conventional four-chain antibodies. See, for example, EP 0 368 684, Ward et al. (Nature 1989 Oct. 12; 341 (6242): 544-6), Holt et al., Trends Biotechnol., 2003, 21(11):484-490; WO 06/030220; and WO 06/003388.

The various binding moieties (such as sdAbs, ligand/receptor domains) in the multispecific or multivalent CARs described herein may be fused to each other via peptide linkers such as any of the peptide linkers described in Section II. In some embodiments, the binding moieties (such as sdAbs, ligand/receptor domains) are directly fused to each other without any peptide linkers. The peptide linkers connecting different binding moieties (such as sdAbs, ligand/receptor domains) may be the same or different. Different domains of the CARs may also be fused to each other via peptide linkers.

Each peptide linker in a CAR may have the same or different length and/or sequence depending on the structural and/or functional features of the sdAbs and/or the various domains (e.g., ligand/receptor domains). Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. For example, in a multivalent or multispecific CAR described herein that comprise sdAbs directed against a multimeric antigen, the length and flexibility of the peptide linkers are preferably such that it allows each sdAb in the multivalent CAR to bind to the antigenic determinant on each of the subunits of the multimer. In some embodiments, a short peptide linker may be disposed between the transmembrane domain and the intracellular signaling domain of a CAR. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids. In some embodiments, the peptide linker comprises the amino acid sequence of any of SEQ ID NOs: 57 and 61-65.

The peptide linker may have a naturally occurring sequence, or a non-naturally occurring sequence. For example, a sequence derived from the hinge region of heavy chain only antibodies may be used as the linker. See, for example, WO1996/34103.

### Transmembrane Domain

The CARs of the present application comprise a transmembrane domain that can be directly or indirectly fused to the extracellular ligand-binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDIIa, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11 d, ITGAE, CD103, ITGAL, CDIIa, LFA-1, ITGAM, CD1 Ib, ITGAX, CD11 c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of α, β, or ζ chain of the T-cell receptor, CD3ζ, CD3ε, CD4, CD5, CD8α, CD9, CD16, CD22, CD27, CD28, CD33, CD37, CD45, CD64, CD80, CD86, CD134, CD137 (4-1BB), CD152, CD154, and PD-1. In some embodiments, the transmembrane domain is derived from CD8α. In some embodiments, the transmembrane domain is derived from CD28.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

### Intracellular Signaling Domain

The CARs of the present application comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, CD3γ, CD3ε, CD3δ, FcRγ (FCER1G), FcRβ (Fc Epsilon RIb), CD5, CD22, CD79a, CD79b, CD66d, Fc gamma RIIa, DAP10, and DAP12.

In some embodiments, the primary intracellular signaling domain is derived from CD3ζ. In some embodiments, the intracellular signaling domain consists of the cytoplasmic signaling domain of CD3ζ. In some embodiments, the primary intracellular signaling domain is a cytoplasmic signaling domain of wildtype CD3ζ.

### Co-Stimulatory Signaling Domain

Many immune effector cells (e.g., T cells) require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains, for example, two copies of the co-stimulatory signaling domain of CD28 or CD137 (4-1BB). In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3ζ) and one or more co-stimulatory signaling domains (e.g., 4-1BB). In some embodiments, the one or more co-stimulatory signaling domains and the primary intracellular signaling domain (such as cytoplasmic signaling domain of CD3ζ) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3ζ). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, IL1A/TNFSF15, TNF-alpha, and TNF RII/TNFRSFlB); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domain is derived from a co-stimulatory molecule selected from the group consisting of CARD11, CD2 (LFA-2), CD7, CD27, CD28, CD30, CD40, CD54 (ICAM-1), CD134 (OX40), CD137 (4-1BB), CD162 (SELPLG), CD258 (LIGHT), CD270 (HVEM, LIGHTR), CD276 (B7-H3), CD278 (ICOS), CD279 (PD-1), CD319 (SLAMF7), LFA-1 (lymphocyte function-associated antigen-1), NKG2C, CDS, GITR, BAFFR, NKp80 (KLRF1), CD160, CD19, CD4, IPO-3, BLAME (SLAMF8), LTBR, LAT, GADS, SLP-76, PAG/Cbp, NKp44, NKp30, NKp46, NKG2D, CD83, CD150 (SLAMF1), CD152 (CTLA-4), CD223 (LAG3), CD273 (PD-L2), CD274 (PD-L1), DAP10, TRIM, ZAP70, a ligand that specifically binds with CD83, and any combination thereof. In some embodiments, the one or more co-stimulatory signaling domain is derived from a co-stimulatory molecule selected from the group consisting of CD27, CD28, 4-1BB, OX40, CD30, CD40, CD3, LFA-1, CD2, CD7, LIGHT, NKG2C, B7-H3 and ligands that specially bind to CD83.

In some embodiments, the intracellular signaling domain in the CAR of the present application comprises a co-stimulatory signaling domain derived from 4-1BB (CD137). In some embodiments, the intracellular signaling domain comprises a cytoplasmic signaling domain of CD3ζ and a co-stimulatory signaling domain of 4-1BB.

In some embodiments, the intracellular signaling domain in the CAR of the present application comprises a co-stimulatory signaling domain derived from CD28. In some embodiments, the intracellular signaling domain comprises a cytoplasmic signaling domain of CD3ζ and a co-stimulatory signaling domain of CD28.

In some embodiments, the intracellular signaling domain in the CAR of the present application comprises a co-stimulatory signaling domain of CD28 and a co-stimulatory signaling domain of CD137. In some embodiments, the intracellular signaling domain comprises a cytoplasmic signaling domain of CD3ζ a co-stimulatory signaling domain of CD28, and a co-stimulatory signaling domain of CD137. In some embodiments, the intracellular signaling domain comprises a polypeptide comprising from the N-terminus to the C-terminus: a co-stimulatory signaling domain of CD28, a co-stimulatory signaling domain of CD137, and a cytoplasmic signaling domain of CD3ζ.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Hinge

The CARs of the present application may comprise a hinge domain that is located between the C-terminus of the extracellular ligand-binding domain and the N-terminus of the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain of the effector molecule can be used.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the CARs described herein. In some embodiments, the hinge domain is at least a portion of a hinge domain of a naturally occurring protein and confers flexibility to the chimeric receptor. In some embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (G×S)ₙ linker, wherein x and n, independently can be an integer between 3 and 12, including 3, 4, 5, 6, 7, 8, 9, 10, 11, 12, or more.

### Signal Peptide

The CARs of the present application may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain. In some embodiments, the signal peptide is derived from CD8α.

ACTR is a chimeric protein that combines the Fc receptor (CD16) with the signal transduction domains (4-1BB/CD3ζ). Engineered T cells bearing the ACTR can bind to a monoclonal antibody which then acts as a bridge to the tumor cells.

In some embodiments, the functional exogenous receptor is a chimeric receptor comprising (a) an extracellular ligand-binding domain that comprises at least one domain derived from a ligand or the extracellular domain of a receptor, wherein the ligand or receptor is a cell surface antigen (e.g., NKG2D, BCMA, IL-3, IL-13); (b) a transmembrane domain; and (c) an intracellular signaling domain.

In some embodiments, the extracellular ligand-binding domain comprises at least one domain derived from a ligand of BCMA, e.g., APRIL or BAFF. In some embodiments, the extracellular ligand-binding domain comprises an antigen-binding fragment (e.g., sdAb) that specifically recognizes one or more epitopes of BCMA.

### Engineered TCRs

In some embodiments, the functional exogenous receptor is an engineered TCR (e.g., an engineered TCR specifically recognizing a tumor antigen, or the tumor antigen-MHC complex) comprising an extracellular ligand-binding domain comprising a Vα and a Vβ derived from a wild type TCR together specifically recognizing an antigen (such as any of the antigens described herein, e.g., tumor antigen), wherein the Vα, the Vβ, or both, comprise a mutation in one or more CDRs relative to the wild type TCR. In some embodiments, the mutation leads to amino acid substitutions, such as conservative amino acid substitutions. In some embodiments, the engineered TCR binds to the same cognate peptide-MHC bound by the wild type TCR. In some embodiments, the engineered TCR binds to the same cognate peptide-MHC with higher affinity compared to that bound by the wild type TCR. In some embodiments, the engineered TCR binds to the same cognate peptide-MHC with lower affinity compared to that bound by the wild type TCR. In some embodiments, the engineered TCR binds to a non-cognate peptide-MHC not bound by the wild type TCR. In some embodiments, the engineered TCR is a single chain TCR (scTCR). In some embodiments, the engineered TCR is a dimeric TCR (dTCR). In some embodiments, the wild type TCR binds HLA-A2. In some embodiments, the engineered TCR further comprises an intracellular signaling domain, such as a primary intracellular signaling domain derived from CD3ζ.

In some embodiments, the engineered TCR comprises an extracellular ligand-binding domain comprising a Vα and a Vβ derived from a wild type TCR together specifically recognizing a tumor antigen or a tumor antigen-MHC complex, wherein the Vα, the Vβ, or both, comprise a mutation in one or more CDRs relative to the wild type TCR. In some embodiments, the engineered anti-tumor antigen TCR has higher binding affinity to the tumor antigen than the wildtype anti-tumor antigen TCR. In some embodiments, the engineered TCR further comprises an intracellular signaling domain, such as a primary intracellular signaling domain derived from CD3ζ.

### IV. Engineered Cells

The present application provides engineered cells that comprise any of the fusion proteins, any of the nucleic acids and/or any of the vectors described herein.

In some embodiments, the engineered cell comprises a) a fusion protein (such as any of the fusion proteins described herein), and b) a functional exogenous receptor (such as any of the functional exogenous receptors described herein). In some embodiments, both the fusion protein and the functional exogenous receptor are expressed on the surface of the engineered cell.

In some embodiments, there is provided an engineered cell (e.g., an immune cell, e.g., a T cell, e.g., a CD8 T cell) comprising (or expressing) a fusion protein comprising: a) a first polypeptide comprising i) a first extracellular domain comprising an extracellular domain of one of TGFβR1 or TGFβR2, ii) a first transmembrane domain, and iii) a first intracellular domain comprising an intracellular domain of one of IL-12Rβ1 or IL-23R; and b) a second polypeptide comprising i) a second extracellular domain comprising an extracellular domain of the other of TGFβR1 or TGFβR2, ii) a second transmembrane domain, and iii) a second intracellular domain comprising an intracellular domain of the other of IL-12Rβ1 or IL-23R. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising an extracellular domain of TGFβR1, ii) a first transmembrane domain comprising a transmembrane domain of TGFβR1, iii) a first intracellular domain comprising an intracellular domain of IL-23R; and b) a second polypeptide comprising i) a second extracellular domain comprising an extracellular domain of TGFβR2, ii) a second transmembrane domain comprising a transmembrane domain of TGFβR2, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising an extracellular domain of TGFβR2, ii) a first transmembrane domain comprising a transmembrane domain of TGFβR2, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of TGFβR1, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising an extracellular domain of TGFβR1, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR2, ii) a second transmembrane domain comprising a transmembrane domain of IL-12Rβ1, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising the amino acid sequence of SEQ ID NO: 3, ii) a first transmembrane domain comprising the amino acid sequence of SEQ ID NO: 5, iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 6, and iii) a second intracellular domain comprising SEQ ID NO: 16. In some embodiments, the first polypeptide further comprises a linker (e.g., a membrane proximal sequence) between the first transmembrane domain and the first intracellular domain, optionally the linker comprises the amino acid sequence of SEQ ID NO: 9. In some embodiments, the second polypeptide further comprises a linker (e.g., a membrane proximal sequence) between the second transmembrane domain and the second intracellular domain, optionally the linker comprises the amino acid sequence of SEQ ID NO: 10. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising the amino acid sequence of SEQ ID NO: 4, ii) a first transmembrane domain comprising the amino acid sequence of SEQ ID NO: 6, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 5, and iii) a second intracellular domain comprising the amino acid sequence of SEQ ID NO: 16. In some embodiments, the first polypeptide further comprises a linker (e.g., a membrane proximal sequence) between the first transmembrane domain and the first intracellular domain, optionally the linker comprises the amino acid sequence of SEQ ID NO: 10. In some embodiments, the second polypeptide further comprises a linker (e.g., a membrane proximal sequence) between the second transmembrane domain and the second intracellular domain, optionally the linker comprises the amino acid sequence of SEQ ID NO: 9. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising the amino acid sequence of SEQ ID NO: 3, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 8, and iii) a second intracellular domain comprising the amino acid sequence of SEQ ID NO: 16. In some embodiments, the first polypeptide further comprises a linker (e.g., a membrane proximal sequence) between the first extracellular domain and the first transmembrane domain, optionally the linker comprises the amino acid sequence of SEQ ID NO: 11. In some embodiments, the second polypeptide further comprises a linker (e.g., a membrane proximal sequence) between the second extracellular domain and the second transmembrane domain, optionally the linker comprises the amino acid sequence of SEQ ID NO: 12. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising i) a first extracellular domain comprising the amino acid sequence of SEQ ID NO: 4, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 8, and iii) a second intracellular domain comprising the amino acid sequence of SEQ ID NO: 16. In some embodiments, the first polypeptide further comprises a linker (e.g., a membrane proximal sequence) between the first extracellular domain and the first transmembrane domain, optionally the linker comprises the amino acid sequence of SEQ ID NO: 11. In some embodiments, the second polypeptide further comprises a linker (e.g., a membrane proximal sequence) between the second extracellular domain and the second transmembrane domain, optionally the linker comprises the amino acid sequence of SEQ ID NO: 12. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) a fusion protein comprising a) a first polypeptide comprising, from N-terminus to C-terminus, i) a first extracellular domain comprising the amino acid sequence of SEQ ID NO: 4, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising: a) a first polypeptide comprising i) a first extracellular domain comprising an extracellular domain of one of TGFβR1 or TGFβR2, ii) a first transmembrane domain, and iii) a first intracellular domain comprising an intracellular domain of one of IL-12Rβ1 or IL-23R; and b) a second polypeptide comprising i) a second extracellular domain comprising an extracellular domain of the other of TGFβR1 or TGFβR2, ii) a second transmembrane domain, and iii) a second intracellular domain comprising an intracellular domain of the other of IL-12Rβ1 or IL-23R; and 2) a functional exogenous receptor comprising an extracellular ligand-binding domain and optionally an intracellular signaling domain. In some embodiments, the functional exogenous receptor is selected from the group consisting of: an engineered T cell receptor (TCR), a chimeric antigen receptor (CAR), a T cell antigen coupler (TAC) or a portion thereof. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1; 2) a functional exogenous receptor comprising a chimeric antigen receptor (e.g., a chimeric antigen receptor that recognizes a tumor antigen, e.g., an anti-GPC3 CAR, e.g., an anti-CD20 CAR). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1; 2) a functional exogenous receptor comprising an engineered TCR (e.g., an engineered TCR that recognizes a tumor antigen). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1; 2) a functional exogenous receptor comprising a T cell antigen coupler (TAC). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising a) a first polypeptide comprising, from N-terminus to C-terminus, i) a first extracellular domain comprising the amino acid sequence of SEQ ID NO: 4, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16; and 2) a functional exogenous receptor comprising a chimeric antigen receptor (e.g., a chimeric antigen receptor that recognizes a tumor antigen, e.g., an anti-GPC3 CAR, e.g., an anti-CD20 CAR). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising a) a first polypeptide comprising, from N-terminus to C-terminus, i) a first extracellular domain comprising the amino acid sequence of SEQ ID NO: 4, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16; and 2) a functional exogenous receptor comprising an engineered TCR (e.g., an engineered TCR that recognizes a tumor antigen). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises (or expresses) 1) a fusion protein comprising a) a first polypeptide comprising, from N-terminus to C-terminus, i) a first extracellular domain comprising the amino acid sequence of SEQ ID NO: 4, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16; and 2) a functional exogenous receptor comprising a T cell antigen coupler (TAC). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises a nucleic acid (or a vector comprising the nucleic acid) comprising a nucleic acid sequence set forth in any of SEQ ID NOs: 28-33 (e.g., SEQ ID NOs: 31-33, e.g., SEQ ID NO: 33). In some embodiments, the engineered cell further comprises a functional exogenous receptor (e.g., a CAR, e.g., an engineered TCR) or a nucleic acid sequence encoding the same. In some embodiments, the nucleic acid comprising a nucleic acid sequence set forth in any of SEQ ID NOs: 28-33 further comprises the nucleic acid sequence encoding the functional exogenous receptor (e.g., a CAR, e.g., an engineered TCR). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

In some embodiments, the engineered cell (e.g., an immune cell, e.g., a T cell, e.g., a CD8 T cell) comprises a nucleic acid comprising 1) a nucleic acid sequence set forth in SEQ ID NO: 33, and 2) a nucleic acid sequence encoding a CAR that recognizes a tumor antigen (e.g., an anti-GPC3 CAR, e.g., an anti-CD20 CAR, e.g., a CAR comprising the amino acid sequence of SEQ ID NO: 36 or 89). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell is an immune cell. In some embodiments, the engineered cell is selected from a group consisting of T cell, NK cell, peripheral blood mononuclear cell (PBMC), hematopoietic stem cell, pluripotent stem cell, an embryonic stem cell, and a combination thereof. In some embodiments, the engineered cell is a T cell. In some embodiments, the T cell is a gamma delta T cell. In some embodiments, the T cell is an alpha beta T cell. In some embodiments, the T cell is an endogenous TCR-deficient T cell. In some embodiments, the engineered cell is a CD8+ T cell. In some embodiments, the engineered cell is a CD4+ T cell. In some embodiments, a signal mediated via IL-23 receptor complex intracellular domain is transmitted from extracellular domain to intracellular domain of the fusion protein upon binding of TGFβ.

### V. Methods of Producing Engineered Cells

One aspect of the present invention provides methods of producing any of the engineered T cells described above. The method generally involves introducing into a cell (e.g., a T cell, referred to herein as “a precursor cell”) a nucleic acid comprising a nucleic acid sequence encoding any of the fusion proteins described herein. In some embodiments, the nucleic acid further comprises a second nucleic acid sequence encoding a functional exogenous receptor (such as an engineered TCR, a CAR, or an ACTR described herein). In some embodiments, the method further comprises introducing into the precursor cell a second nucleic acid comprising a nucleic acid sequence encoding a functional exogenous receptor (such as an engineered TCR, a CAR, or an ACTR described herein). In some embodiments, the precursor cell is an immune cell. In some embodiments, the precursor cell is selected from a group consisting of T cell, NK cell, peripheral blood mononuclear cell (PBMC), hematopoietic stem cell, pluripotent stem cell, an embryonic stem cell, and a combination thereof.

In some embodiments, the precursor cell (e.g., the precursor T cell) are derived from the blood, bone marrow, lymph, or lymphoid organs, are cells of the immune system, such as cells of the innate or adaptive immunity. In some aspects, the cells are human cells.

In some embodiments, the precursor (.e.g, the precursor T cells) are derived from cell lines, e.g., T cell lines. The cells in some embodiments are obtained from a xenogeneic source, for example, from mouse, rat, non-human primate, and pig.

In some embodiments, the precursor T cells are CD4+/CD8−, CD4−/CD8+, CD4+/CD8+, CD4−/CD8−, or combinations thereof. In some embodiments, the T cell is a natural killer T (NKT) cell. In some embodiments, the precursor T cell is an engineered T cell, such as any of the functional exogenous receptor (e.g., engineered TCR or CAR) described herein. In some embodiments, the precursor T cells produce IL-2, TFN, and/or TNF upon expressing the functional exogenous receptor (e.g., engineered TCR, CAR) described herein and binding to the target cells, such as tumor cells expressing a tumor antigen (e.g., GPC3). In some embodiments, the CD8+ T cells lyse antigen-specific target cells upon expressing the functional exogenous receptor (e.g., engineered TCR, CAR) described herein and binding to the target cells.

In some embodiments, the precursor cells (e.g., the precursor T cells) are differentiated from a stem cell, such as a hematopoietic stem cell, a pluripotent stem cell, an iPS, or an embryonic stem cell.

In some embodiments, the fusion protein and/or the functional exogenous receptor (e.g., engineered TCR, CAR) are introduced to the precursor cells (e.g., the precursor T cells) by transfecting any one of the nucleic acids or any one of the vectors (e.g., non-viral vectors and viral vectors such as lentiviral vectors) described herein. In some embodiments, the functional exogenous receptor (e.g., engineered TCR, CAR) is introduced to the precursor cells (e.g., the precursor T cells) by inserting proteins into the cell membrane while passing cells through a microfluidic system, such as CELL SQUEEZE® (see, for example, U.S. Patent Application Publication No. 20140287509).

Methods of introducing vectors (e.g., viral vectors) or isolated nucleic acids into a mammalian cell are known in the art. The vectors described herein can be transferred into a cell (e.g., a T cell) by physical, chemical, or biological methods.

Physical methods for introducing the vector (e.g., viral vectors) into a cell (e.g., a T cell) include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001) Molecular Cloning: A Laboratory Manual, Cold Spring Harbor Laboratory, New York. In some embodiments, the vector (e.g., viral vector) is introduced into the cell by electroporation.

Biological methods for introducing the vector into a cell (e.g., a T cell) include the use of DNA and RNA vectors. Viral vectors have become the most widely used method for inserting genes into mammalian, e.g., human cells.

Chemical means for introducing the vector (e.g., viral vector) into a cell (e.g., a T cell) include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the fusion proteins and/or functional exogenous receptors (e.g., engineered TCR, CAR) described herein may be prepared by a conventional method (e.g., in vitro transcription) and then introduced into the cell (e.g., the T cell) via known methods such as mRNA electroporation. See, e.g., Rabinovich et al., Human Gene Therapy 17:1027-1035.

In some embodiments, the transduced or transfected cell is propagated ex vivo after introduction of the vector or isolated nucleic acid. In some embodiments, the transduced or transfected cell is cultured to propagate for at least about any of 1 day, 2 days, 3 days, 4 days, 5 days, 6 days, 7 days, 10 days, 12 days, or 14 days. In some embodiments, the transduced or transfected cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding any of the fusion proteins and/or functional exogenous receptors (e.g., engineered TCR, CAR) described herein in the engineered cells (e.g., engineered T cells), include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots), Fluorescence-activated cell sorting (FACS), or Magnetic-activated cell sorting (MACS) (also see Example section).

Thus in some embodiments, there is provided a method of producing an engineered cell (e.g., an engineered T cell, an allogeneic T cell, or an endogenous TCR-deficient T cell), comprising: introducing into a precursor cell a nucleic acid comprising a nucleic acid sequence encoding a fusion protein described herein. In some embodiments, the nucleic acid further comprises a second nucleic acid sequence encoding any of the functional exogenous receptor comprising an extracellular ligand-binding domain and an intracellular signaling domain (e.g., engineered TCR, CAR). In some embodiments, the precursor cell comprises a second nucleic acid encoding a functional exogenous receptor comprising an extracellular ligand-binding domain and an intracellular signaling domain (e.g., engineered TCR, CAR). In some embodiments, the method further comprises introducing into the precursor T cell a second nucleic acid encoding a functional exogenous receptor comprising an extracellular ligand-binding domain and an intracellular signaling domain. In some embodiments, the first nucleic acid and the second nucleic acid are introduced into the T cell sequentially. Thus in some embodiments, there is provided a method of producing an engineered cell (e.g., a T cell, an allogeneic T cell, or an endogenous TCR-deficient T cell), comprising: introducing into a precursor cell a nucleic acid encoding a) a fusion protein (such as any of the fusion proteins described herein, and b) a functional exogenous receptor (such as any of the functional exogenous receptor described herein. In some embodiments, there is provided a method of producing an engineered cell (e.g., a T cell, an allogeneic T cell, or an endogenous TCR-deficient T cell), comprising: introducing into a precursor cell a first nucleic acid encoding a fusion protein, then introducing into the precursor cell a second nucleic acid encoding a functional exogenous receptor comprising an extracellular ligand-binding domain and an intracellular signaling domain. In some embodiments, the first nucleic acid and the second nucleic acid are introduced into the T cell simultaneously. In some embodiments, the first nucleic acid and the second nucleic acid are on separate vectors. In some embodiments, the first nucleic acid and the second nucleic acid are on the same vector. In some embodiments, the first nucleic acid and the second nucleic acid are operably linked to different promoters. In some embodiments, the first nucleic acid is upstream of the second nucleic acid. In some embodiments, the first nucleic acid is downstream of the second nucleic acid.

In some embodiments, there is provided a method of producing an engineered cell (e.g., an engineered T cell, an engineered allogeneic T cell or an engineered endogenous TCR-deficient T cell), comprising introducing into a precursor cell a vector (e.g., viral vector such as a lentiviral vector) from upstream to downstream: a promoter (e.g., EF1-α), a first nucleic acid encoding the functional exogenous receptor (e.g., an engineered TCR or a CAR), and a second nucleic acid encoding a fusion protein as described herein. In some embodiments, the first nucleic acid and the second nucleic acid are separated by a third nucleic acid encoding a multicistronic element (e.g., a 2A self-cleaving peptide selected from the group consisting of T2A, P2A, E2A, or F2A).

In some embodiments, there is provided a method of producing an engineered cell (e.g., an engineered T cell, an engineered allogeneic T cell or an engineered endogenous TCR-deficient T cell), comprising introducing into a precursor cell a vector (e.g., viral vector such as a lentiviral vector) from upstream to downstream: a promoter (e.g., EF1-α), a first nucleic acid encoding a fusion protein such as any of the fusion proteins described herein, and a second nucleic acid encoding the functional exogenous receptor (e.g., an engineered TCR or a CAR). In some embodiments, the first nucleic acid and the second nucleic acid are separated by a third nucleic acid encoding a multicistronic element (e.g., a 2A self-cleaving peptide selected from the group consisting of T2A, P2A, E2A, or F2A).

In some embodiments, the engineered cell is a T cell, and the endogenous TCR locus in the T cell is modified by a CRISPR-Cas system, thereby generating an endogenous TCR-deficient T cell. See e.g., WO 2020/020359.

In some embodiments, the methods described herein further comprise removing alpha beta T cells or enriching gamma delta T cells.

In some embodiments, the promoter is selected from the group consisting of a Rous Sarcoma Virus (RSV) promoter, a Simian Virus 40 (SV40) promoter, a cytomegalovirus immediate early gene promoter (CMV IE), an elongation factor 1 alpha promoter (EF1-α), a phosphoglycerate kinase-1 (PGK) promoter, a ubiquitin-C (UBQ-C) promoter, a cytomegalovirus enhancer/chicken beta-actin (CAG) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter, an NFAT promoter, a TETON® promoter, and an NFκB promoter. In some embodiments, the promoter is EF1-α.

In some embodiments, the multicistronic element comprises P2A, T2A, E2A, F2A, BmCPV 2A, BmIFV 2A, IRES, (GS)ₙ, (GSGGS)ₙ, (GGGS)ₙ, (GGGGS)ₙ, or a combination thereof, wherein n is an integer of at least one.

In some embodiments, the vector is a viral vector. In some embodiments, the viral vector selected from the group consisting of adenoviral vector, adeno-associated virus vector, retroviral vector, vaccinia vector, lentiviral vector, herpes simplex viral vector, and derivatives thereof. In some embodiments, the vector is a non-viral vector, such as episomal expression vector, Enhanced Episomal Vector (EEV), PiggyBac Transposase Vector, or Sleeping Beauty (SB) transposon system. In some embodiments, the functional exogenous receptor is an engineered TCR. In some embodiments, the functional exogenous receptor is a non-TCR receptor, such as CAR (e.g., anti-antigen CAR, ligand/receptor CAR, ACTR).

In some embodiments, the method further comprises formulating the engineered cells expressing the fusion protein with at least one pharmaceutically acceptable carrier. In some embodiments, the method further comprises administering to an individual an effective amount of the engineered cells expressing the fusion protein, or an effective amount of the pharmaceutical formulation comprising the engineered cells (e.g., T cells) expressing the fusion protein and at least one pharmaceutically acceptable carrier. In some embodiments, the individual has cancer. In some embodiments, the individual is a human.

### Source of Cells, Cell Preparation and Culture

Prior to expansion and genetic modification of the cells (e.g., T cells), a source of cells is obtained from an individual. For example, immune cells (e.g., T cells) can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of cell lines that have desired cells (e.g., T cells) available in the art, may be used. In some embodiments, the cells (e.g., the T cells) can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, the cell (the T cell) is provided from an umbilical cord blood bank, a peripheral blood bank, or derived from an induced pluripotent stem cell (iPSC), multipotent and pluripotent stem cell, or a human embryonic stem cell. In some embodiments, the cells (e.g., the T cells) are derived from cell lines. The cells (e.g., the T cells) in some embodiments are obtained from a xenogeneic source, for example, from mouse, rat, non-human primate, and pig. In some embodiments, the cells (e.g., the T cells) are human cells. In some aspects, the cells (e.g., the T cells) are primary cells, such as those isolated directly from a subject and/or isolated from a subject and frozen. In some embodiments, the cells include one or more subsets of T cells, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some cases, the cell (e.g., the T cell) is allogeneic in reference to one or more intended recipients.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of cells (e.g., T cells) and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present application is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Whether prior to or after genetic modification of the T cells with the fusion protein or exogenous receptor (e.g., engineered TCR, CAR) described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the T cells are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, the methods include assessing expression of one or more markers on the surface of the modified cells or cells to be engineered. In one embodiment, the methods include assessing surface expression of TCR or CD3R, for example, by affinity-based detection methods such as by flow cytometry. In some aspects, where the method reveals surface expression of the antigen or other marker, the gene encoding the antigen or other marker is disrupted or expression otherwise repressed for example, using the methods described herein.

### VI. Pharmaceutical Compositions

Further provided by the present application are pharmaceutical compositions comprising any one of the engineered cells (e.g., engineered T cells) expressing a fusion protein described herein and/or a functional exogenous receptor (e.g., engineered TCR, CAR such as anti-BCMA CAR) described herein, and a pharmaceutically acceptable carrier. Pharmaceutical compositions can be prepared by mixing a plurality of engineered cells with optional pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1 to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/mL to about 1.0 mg/mL, preferably about 0.07 mg/mL to about 0.2 mg/mL.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly (2-hydroxyethyl-methacrylate), or poly(vinylalcohol)), polylactides (U.S. Pat. No. 3,773,919), copolymers of L-glutamic acid and. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions described herein may also contain more than one active compound or agent as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

### VII. Methods of Treatment

The present application further provides methods of treating a disease or condition in an individual, comprising administering to the individual a pharmaceutical composition comprising an effective amount of engineered cells described herein. In some embodiments, the disease or condition is associated with immunosuppression. In some embodiments, the diseased tissue has a higher expression level of TGFβ (e.g., a higher expression of TGFβ mRNA or TGFβ protein) than a corresponding tissue in an individual without the disease or condition. In some embodiments, the diseased tissue has a higher expression level of TGFβR (e.g., a higher expression of TGFβR1/TGFβR2 mRNA or TGFβR1/TGFβR2 protein) than a corresponding tissue in an individual without the disease or condition. In some embodiments, the disease or condition is a cancer. In some embodiments, the cancer is a solid tumor. In some embodiments, the disease or condition is an infectious disease or a condition associated with an infection.

The present application also provides methods of reducing an immunosuppression signal in a diseased tissue in an individual, comprising administering to the individual a pharmaceutical composition comprising an effective amount of engineered cells described herein. In some embodiments, the reducing the immunosuppression signal comprises decreasing signaling through TGFβR. In some embodiments, the diseased tissue has a higher expression level of TGFβ (e.g., a higher expression of TGFβ mRNA or TGFβ protein) than a corresponding tissue in an individual without the disease or condition. In some embodiments, the diseased tissue has a higher expression level of TGFβR (e.g., a higher expression of TGFβR1/TGFβR2 mRNA or TGFβR1/TGFβR2 protein) than a corresponding tissue in an individual without the disease or condition. In some embodiments, the disease or condition is a cancer. In some embodiments, the cancer is a solid tumor. In some embodiments, the disease or condition is an infectious disease or a condition associated with an infection.

In some embodiments, the method further comprises assessing the level of TGFβ or TGFβ receptor (e.g., TGFβR1 and/or TGFβR2) in diseased tissue prior to the treatment. In some embodiments, the method further comprises selecting an individual for treatment based upon the level of TGFβ or TGFβ receptor (e.g., TGFβR1 and/or TGFβR2) in diseased tissue. In some embodiments, the individual is selected for treatment when the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the individual is selected for treatment when the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual).

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising an extracellular domain of TGFβR1, ii) a first transmembrane domain comprising a transmembrane domain of TGFβR1, iii) a first intracellular domain comprising an intracellular domain of IL-23R; and b) a second polypeptide comprising i) a second extracellular domain comprising an extracellular domain of TGFβR2, ii) a second transmembrane domain comprising a transmembrane domain of TGFβR2, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cells further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising an extracellular domain of TGFβR2, ii) a first transmembrane domain comprising a transmembrane domain of TGFβR2, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of TGFβR1, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cells further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising an extracellular domain of TGFβR1, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR2, ii) a second transmembrane domain comprising a transmembrane domain of IL-12Rβ1, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cells further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cells further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) 1) a fusion protein comprising a) a first polypeptide comprising i) a first extracellular domain comprising an extracellular domain of TGFβR2, ii) a first transmembrane domain comprising a transmembrane domain of IL-23R, and iii) a first intracellular domain comprising an intracellular domain of IL-23R; and b) a second polypeptide comprising i) a second extracellular domain comprising an extracellular domain of TGFβR1, ii) a second transmembrane domain comprising a transmembrane domain of IL-12Rβ1, and iii) a second intracellular domain comprising an intracellular domain of IL-12Rβ1; and 2) a CAR that targets a tumor antigen (e.g., GPC3 or BCMA). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising the amino acid sequence of SEQ ID NO: 3, ii) a first transmembrane domain comprising the amino acid sequence of SEQ ID NO: 5, iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 6, and iii) a second intracellular domain comprising SEQ ID NO: 16. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising the amino acid sequence of SEQ ID NO: 4, ii) a first transmembrane domain comprising the amino acid sequence of SEQ ID NO: 6, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 5, and iii) a second intracellular domain comprising the amino acid sequence of SEQ ID NO: 16. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising the amino acid sequence of SEQ ID NO: 3, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 8, and iii) a second intracellular domain comprising the amino acid sequence of SEQ ID NO: 16. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising i) a first extracellular domain comprising the amino acid sequence of SEQ ID NO: 4, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 8, and iii) a second intracellular domain comprising the amino acid sequence of SEQ ID NO: 16. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises (or expresses) a fusion protein comprising a) a first polypeptide comprising, from N-terminus to C-terminus, i) a first extracellular domain comprising the amino acid sequence of SEQ ID NO: 4, ii) a first linker comprising the amino acid sequence of SEQ ID NO: 11, iii) a first transmembrane domain comprising the amino acid sequence of SEQ ID NO: 7, and iv) a first intracellular domain comprising the amino acid sequence of SEQ ID NO: 15; and b) a second polypeptide comprising, from N-terminus to C-terminus, i) a second extracellular domain comprising the amino acid sequence of SEQ ID NO: 3, ii) a second linker comprising the amino acid sequence of SEQ ID NO: 12, ii) a second transmembrane domain comprising the amino acid sequence of SEQ ID NO: 8, and iv) a second intracellular domain comprising SEQ ID NO: 16. In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell further comprises a functional exogenous receptor (e.g., a CAR, e.g., an engineered TCR) or a nucleic acid sequence encoding the same. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises a nucleic acid comprising a nucleic acid sequence set forth in any of SEQ ID NOs: 28-33 (e.g., SEQ ID NOs: 31-33, e.g., SEQ ID NO: 33). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the engineered cell further comprises a functional exogenous receptor (e.g., a CAR, e.g., an engineered TCR) or a nucleic acid sequence encoding the same. In some embodiments, the nucleic acid comprising a nucleic acid sequence set forth in any of SEQ ID NOs: 28-33 further comprises the nucleic acid sequence encoding the functional exogenous receptor (e.g., a CAR, e.g., an engineered TCR). In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, there is provided a method of treating a disease or condition (e.g., a cancer, a solid tumor, a hematological cancer, or an infectious disease) in an individual, comprising administering a composition (e.g., a pharmaceutical composition) comprising an effective amount of engineered cells (e.g., an immune cell, e.g., a T cell) that comprises a nucleic acid comprising 1) a nucleic acid sequence set forth in SEQ ID NO: 33, and 2) a nucleic acid sequence encoding a CAR that recognizes a tumor antigen (e.g., an anti-GPC3 CAR, e.g., an anti-CD20 CAR, e.g., a CAR comprising the amino acid sequence of SEQ ID NO: 36 or 89). In some embodiments, the engineered cell is an allogeneic cell (e.g., allogeneic T cell). In some embodiments, the engineered cell is an autologous cell (e.g., autologous T cell). In some embodiments, the engineered cell is a gamma delta T cell. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, engineered cells are parentally (e.g., intravenously) administered into the individual. In some embodiments, the individual is a human.

In some embodiments, the engineered cells are autologous to the individual.

In some embodiments, the engineered cells are allogeneic to the individual.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old).

In some embodiments, the diseased tissue exhibits a high level of immunosuppression. In some embodiments, the diseased tissue has a TGFβ level (e.g., an average TGFβ level) at least about 10%, 20%, 30%, 40%, 50%, 60%, 70%, 80%, or 90% higher than that in a reference tissue (e.g., a corresponding tissue in a healthy individual). In some embodiments, the diseased tissue has a TGFβR level (e.g., an average TGFβR1 and/or an average TGFβR2 level) at least about 10%, 20%, 30%, 40%, 50%, 60%, 70%, 80%, or 90% higher than that in a reference tissue (e.g., a corresponding tissue in a healthy individual). In some embodiments, the diseased tissue has a PD-1 or PD-L1 level (e.g., an average PD-1 or PD-L1 level) at least about 10%, 20%, 30%, 40%, 50%, 60%, 70%, 80%, or 90% higher than that in a reference tissue (e.g., a corresponding tissue in a healthy individual).

Administration of the pharmaceutical compositions may be carried out in any convenient manner, including by injection, ingestion, transfusion, implantation or transplantation. The compositions may be administered to a patient transarterially, subcutaneously, intradermally, intratumorally, intranodally, intramedullary, intramuscularly, intravenously, or intraperitoneally. In some embodiments, the pharmaceutical composition is administered systemically. In some embodiments, the pharmaceutical composition is administered to an individual by infusion, such as intravenous infusion. Infusion techniques for immunotherapy are known in the art (see, e.g., Rosenberg et al., New Eng. J. of Med. 319: 1676 (1988)). In some embodiments, the pharmaceutical composition is administered to an individual by intradermal or subcutaneous injection. In some embodiments, the compositions are administered by intravenous injection. In some embodiments, the compositions are injected directly into a tumor, or a lymph node. In some embodiments, the pharmaceutical composition is administered locally to a site of tumor, such as directly into tumor cells, or to a tissue having tumor cells.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

In some embodiments, wherein the pharmaceutical composition comprises any one of the engineered cells (e.g., engineered T cells) expressing the fusion protein and/or the functional exogenous receptor (e.g., engineered TCR, CAR such as anti-GPC3 CAR) described herein, the pharmaceutical composition is administered at a dosage of at least about any of 10⁴, 10⁵, 10⁶, 10⁷, 10⁸, or 10⁹ cells/kg of body weight of the individual. In some embodiments, the pharmaceutical composition is administered at a dosage of any of about 10⁴ to about 10⁵, about 10⁵ to about 10⁶, about 10⁶ to about 10⁷, about 10⁷ to about 10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight of the individual. In some embodiments, the pharmaceutical composition is administered at a dose of at least about any 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷ cells/kg or more. In some embodiments, the pharmaceutical composition is administered at a dose of about 3×10⁵ to about 7×10⁶ cells/kg, or about 3×10⁶ cells/kg.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

Moreover, dosages may be administered by one or more separate administrations, or by continuous infusion. In some embodiments, the pharmaceutical composition is administered in split doses, such as about any one of 2, 3, 4, 5, or more doses. In some embodiments, the split doses are administered over about a week. In some embodiments, the dose is equally split. In some embodiments, the split doses are about 20%, about 30% and about 50% of the total dose. In some embodiments, the interval between consecutive split doses is about 1 day, 2 days, 3 days or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, the disease or condition is a cancer. In some embodiments, the disease or condition is a solid tumor. Examples of cancers that may be treated by the methods described herein include, but are not limited to, adenocortical carcinoma, agnogenic myeloid metaplasia, anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, lip and oral cavity cancer, oral cancer, liver cancer, lung cancer (e.g., small cell lung cancer, non-small cell lung cancer, adenocarcinoma of the lung, and squamous carcinoma of the lung), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal carcinoma, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, pulmonary lymphangiomyomatosis, rectal cancer, renal carcinoma, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thyroid cancer, tuberous sclerosis, urethral cancer, vaginal cancer, vulvar cancer, Wilms' tumor, abnormal vascular proliferation associated with phakomatoses, edema (such as that associated with brain tumors), and Meigs' syndrome.

In some embodiments, the engineered cells exhibit decreased TGFβ downstream pathway signaling (e.g., phosphorylation of Smad2) by at least 10%, 20%, 30%, 40%, 50%, 60%, 70%, 80% or 90% as compared to corresponding cells without the fusion protein upon exposure to TGFβ. In some embodiments, the engineered cells exhibit increased IL-23R downstream signaling (e.g., phosphorylation of Stat3 and/or Stat4) by at least 10%, 20%, 30%, 40%, 50%, 60%, 70%, 80% or 90% as compared to corresponding cells without the fusion protein upon exposure to TGFβ.

In some embodiments, the engineered cells exhibit increased cell viability by at least 10%, 20%, 30%, 40%, 50%, 60%, 70%, 80% or 90% as compared to corresponding cells without the fusion protein upon exposure to TGFβ. In some embodiments, the engineered cells exhibit a lower PD-1 expression (e.g., at least 10%, 20%, 30%, 40%, 50%, 60%, 70%, 80% or 90% lower) than corresponding cells without the fusion protein upon exposure to TGFβ (e.g., repeated or continuous exposure to TGFβ, e.g., an over 100-hour, 200-hour, or 300-hour exposure to TGFβ). In some embodiments, the engineered cells exhibit an increased proliferation (e.g., by at least 10%, 20%, 30%, 40%, 50%, 60%, 70%, 80% or 90%) than corresponding cells without the fusion protein upon exposure to TGFβ (e.g., repeated or continuous exposure to TGFβ, e.g., an over 100-hour, 200-hour, or 300-hour exposure to TGFβ). In some embodiments, the engineered cells exhibit an increased cytotoxicity (e.g., by at least 10%, 20%, 30%, 40%, 50%, 60%, 70%, 80% or 90%) than corresponding cells without the fusion protein upon exposure to TGFβ (e.g., repeated or continuous exposure to TGFβ, e.g., an over 100-hour, 200-hour, or 300-hour exposure to TGFβ).

In some embodiments, the engineered cells exhibit better anti-tumor effect in vivo. In some embodiments, the engineered cells exhibit a tumor volume reduction at least 10%, 20%, 30%, 40%, 50%, 60%, 70%, 80% or 90% more than the corresponding cells without the fusion protein upon exposure to TGFβ.

In some embodiments, the treatment effect comprises causing an objective clinical response in the individual. In some embodiments, Stringent Clinical Response (sCR) is obtained in the individual. In some embodiments, the treatment effect comprises causing disease remission (partial or complete) in the individual. In some the clinical remission is obtained after no more than about any one of 6 months, 5 months, 4 months, 3 months, 2 months, 1 months or less after the individual receives the pharmaceutical composition. In some embodiments, the treatment effect comprises preventing relapse or disease progression of the cancer in the individual. In some embodiments, the relapse or disease progression is prevented for at least about 6 months, 1 year, 2 years, 3 years, 4 years, 5 years or more. In some embodiments, the treatment effect comprises prolonging survival (such as disease free survival) in the individual. In some embodiments, the treatment effect comprises improving quality of life in an individual. In some embodiments, the treatment effect comprises inhibiting growth or reducing the size of a solid or lymphatic tumor.

In some embodiments, the size of the solid or lymphatic tumor is reduced for at least about 10% (including for example at least about any of 20%, 30%, 40%, 60%, 70%, 80%, 90%, or 100%). In some embodiments, a method of inhibiting growth or reducing the size of a solid or lymphatic tumor in an individual is provided. In some embodiments, the treatment effect comprises inhibiting tumor metastasis in the individual. In some embodiments, at least about 10% (including for example at least about any of 20%, 30%, 40%, 60%, 70%, 80%, 90%, or 100%) metastasis is inhibited. Metastasis can be assessed by any known methods in the art, such as by blood tests, bone scans, x-ray scans, CT scans, PET scans, and biopsy.

In some embodiments, the engineered cell compositions of the invention are administered in combination with a second, third, or fourth agent (including, e.g., an antineoplastic agent, a growth inhibitory agent, a cytotoxic agent, or a chemotherapeutic agent) to treat diseases or disorders (e.g., a cancer, e.g., a solid tumor). In some embodiments, the methods of treating an infectious disease described herein further comprises administering to the individual an anti-infectious disease agent. In some embodiments, the second anti-infectious agent is administered simultaneously with the engineered cells. In some embodiments, the second anti-infectious agent is administered sequentially with (e.g., prior to or after) the administration of the engineered cells.

### VIII. Kits and Articles of Manufacture

Further provided are kits, unit dosages, and articles of manufacture comprising any one of the engineered cells (e.g., engineered T cells) expressing a fusion protein and/or a functional exogenous receptor (e.g., an engineered TCR or a CAR, e.g., an anti-GPC3 CAR, e.g., an anti-CD20 CAR) described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as a disease or condition associated with immunosuppression) as described herein, or reducing reducing an immunosuppression signal in a diseased tissue in an individual, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples described herein are not intended to represent that the experiments below are all or the only experiments performed. Efforts have been made to ensure accuracy with respect to numbers used (for example, amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1. Generation of T Cells Expressing the TGFβ Receptor and IL-23 Receptor Fusion Protein (TGB23)

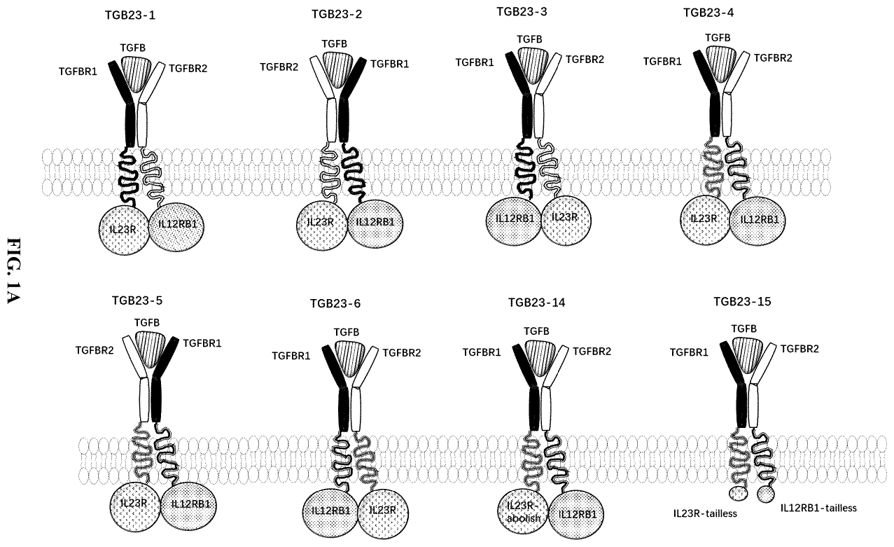

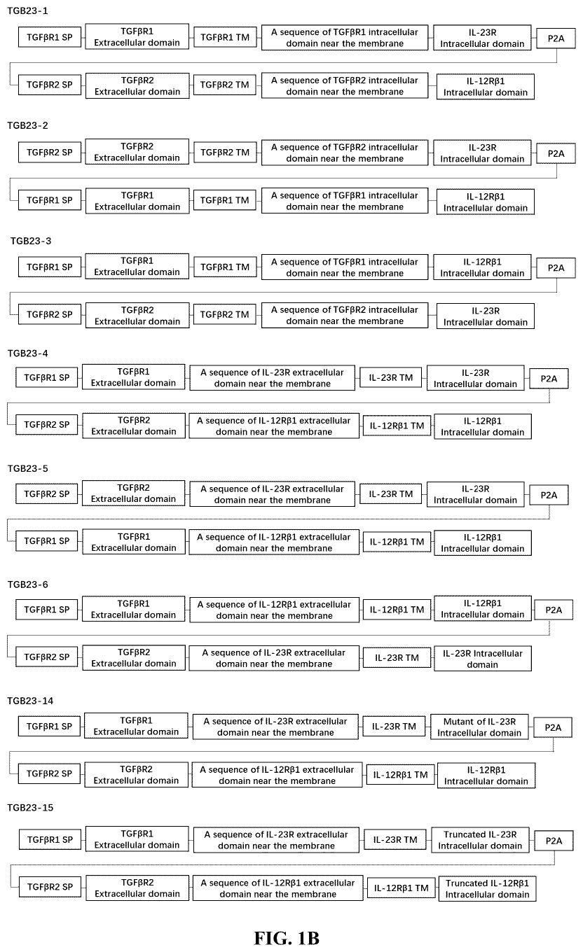

The structures of the exemplary TGFβ receptor and IL-23 receptor fusion proteins (herein after named as TGB23) are shown in FIG. 1A and FIG. 1B. In order to convert a TGFβ receptor (TGFβR) signal to induce IL-23 receptor (IL-23R) signal after exposure to TGFβ, the intracellular domains of TGFβ receptor 1 (TGFβR1) and TGFβ receptor 2 (TGFβR2) were replaced with an IL-23R intracellular domain or an IL-12Rβ1 intracellular domain, respectively. Wild type TGFβR (wtTGFβR) with human natural isoforms of TGFβR1 (SEQ ID NO: 66) and TGFβR2 (SEQ ID NO: 70) was also generated.

### 1. Preparation of Lentiviral Expression Vectors Containing TGB23 Fusion Proteins

Nucleotide sequences of TGB23 fusion protein were cloned into lentiviral expression vector at cloning site 5′-EcoRI and 3′-XbaI. The lentivirus packaging plasmid mixture containing pMDLg.pRRE, pRSV-REV and pMD2.G were pre-mixed with the vectors expressing TGB23 constructs shown in FIG. 1 at a pre-optimized ratio with polyetherimide, and then incubated at 25° C. for 5 min. HEK293T cells were added into the transduction mix. Afterwards, cells were incubated overnight in a cell incubator with 5% CO₂ at 37° C. The supernatants were collected after centrifuged at 4° C. and 3000 g for 15 min, sequentially concentrated by ultra-centrifugation. The supernatants were discarded and the virus pellets were rinsed with RPMI 1640 medium. The viruses were liquated properly, and stored at −80° C. The virus titer was determined by a titration method via transduction of Chinese hamster ovarian cell line.

### 2. T Cell Purification and Activation

Human T cells were isolated from PBMCs (TPCS #A19K470047) using Pan T cell isolation kit (Miltenyi #130-096-535), following manufacturer's protocol as described below. Cell number was determined and cell suspension was centrifuged at 300 g for 10 minutes, then the cell pellet was re-suspended in 40 μL buffer containing total 10⁷ cells. 10 μL of Pan T Cell Biotin-Antibody Cocktail was added per 10⁷ total cells, mixed thoroughly and incubated for 5 minutes in the 4° C. 30 μL of buffer and 20 μL of Pan T Cell MicroBead Cocktail was then added per 10⁷ cells, mixed well and incubated for 10 minutes in the 4° C. A minimum of 500 μL was required for magnetic separation. For magnetic separation, an LS column was placed in the magnetic field of a suitable MACS Separator. The column was prepared by rinsing with 3 mL of buffer. The cell suspension was then applied onto the column, and flow-through containing the unlabeled cells was collected, which represented the enriched T cell fractions. The column was then washed with 3 mL of buffer for collecting unlabeled cells that pass through. These unlabeled cells were combined with the flow-through from previous step. The pooled enriched T cells were centrifuged and re-suspended in RPMI 1640 medium with 10% FBS and 20 ng/mL TGF-β1 (R&D #240-B-010).

The purified T cells were subsequently pre-activated for 48 hours with T Cell Activation/Expansion Kit (Miltenyi #130-091-441) according to manufacturer's protocol in which anti-CD2/CD3/CD28 MACSiBead particles were added at a bead-to-cell ratio 1:2.

### 3. T Cell Transduction and TGF-β Receptor Detection

The pre-activated T cells were re-suspended at 1×10⁶ cells/mL in 24-well plates and then transduced with lentivirus vector expressing TGB23 constructs (multiplicity of infection=10) and incubated overnight. On the next day, 1.5 mL RPMI 1640 medium containing 10% FBS and 20 ng/mL TGFβ1 was added to the transduced cells.

The expressions of TGB23 constructs were measured by FACS. Cells were re-suspended with 100 μL DPBS containing TGF-β RII antibody (Miltenyi Biotec #130-115-024) and incubated for 30 min at 4° C. Un-transduced T cells (UnT) were used as a negative control. As shown in FIG. 2, the positive rates of TGB23-1, TGB23-2, TGB23-3, TGB23-4, TGB23-5, TGB23-6, wtTGFβR (wild type TGFβR), TGB23-14, and TGB23-15 were 8.06%, 3.16%, 6.30%, 5.01%, 9.47%, 46.5%, 6.78%, 4.37%, and 2.76%. The results indicate that the TGB23-6 fusion protein/construct has the best potency of expression compared to the other constructs.

### 4. Cell Viability of T Cells Expressing TGB23

0.5×10⁶ T cells expressing TGB23 were cultured with RPMI 1640 medium containing 10% FBS and 20 ng/mL TGFβ1 after transduction. TGB23 T cells were re-suspended, and 20 μL cell suspensions were taken for Trypan Blue staining. The cell viability was recorded and cell number was counted by T4 (Nexcelom) cell counter on day 3, day 4, day 5 after transduction.

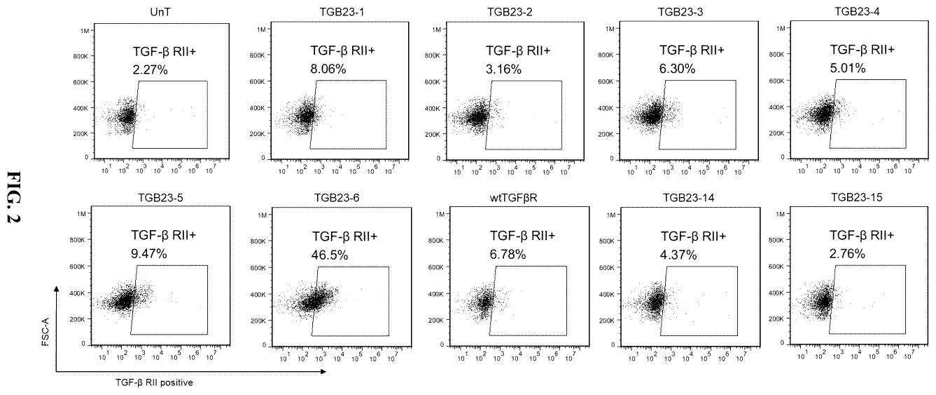

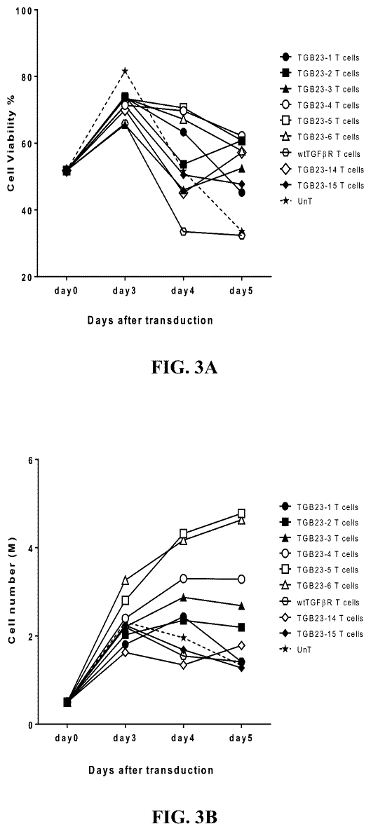

The cell viability and the cell number of UnT and TGB23 T cells are shown in FIGS. 3A-3B. Five days after transduction, the treatment of TGFβ1 led to the decrease in the cell viability of UnT (from 51.8% to 33.6%) and wtTGFβR T cells (from 51.8% to 32.4%), respectively. TGB23 T cells have capability to counteract the function of TGFβ1 at different extent. Among them, TGB23-2, TGB23-4, TGB23-5, TGB23-6 and TGB23-14 had higher viability on day 5 than that on day 0.5 days after transduction and culture in the presence of TGFβ 1, T cell proliferation was also inhibited (FIG. 3B). The total number of UnT and wtTGFβR T cells increased slightly. Although TGB23-14 (with a mutant of IL-23R intracellular domain non-functional, SEQ ID NO:26) and TGB23-15 (with truncated IL-23R and truncated IL-12Rβ1 intracellular domain both non-functional, SEQ ID NO:27) constructs could counteract the function of TGFβ1 at different extent in theory, the absence of the function from intracellular signaling domain provided little help to T cell proliferation, which could be observed from the similar cell number as UnT and wtTGFβR T cells, 5 days after transduction. T cells transduced with TGB23-2, TGB23-3, TGB23-4, TGB23-5 and TGB23-6 increased by more than 4 folds from day 0 to day 5. These results suggest that these TGB23 T cells bound to TGFβ, and the signaling through intracellular IL23 receptor provided T cell a pro-proliferation signal. Among them, TGB23-5 and TGB23-6 have the most potent to promote T cell proliferation in the presence of TGFβ1.

### Example 2. Generation of CAR-T Cells Expressing TGB-23 Fusion Proteins

To verify that TGB23 constructs provide CAR-T cells benefits against target cells under existence of TGFβ, GPC3 CAR-T cells co-expressing TGB23-6 fusion proteins were produced.

### 1. Preparation of Lentiviral Expression Vectors Containing Chimeric Antigen Receptors (CAR) and TGB23-6

The CAR backbone comprising the polypeptide from N-terminus to the C-terminus: a CD8α hinge domain (SEQ ID NO: 40), a CD8α transmembrane domain (SEQ ID NO: 41), a CD137 co-stimulatory signaling domain (SEQ ID NO: 42), and a CD3ζ intracellular signaling domain (SEQ ID NO: 43), was chemically synthesized and cloned into a pre-modified lentiviral vector (pLSINK-BBzBB) downstream and operably linked to a constitutive hEF1α promoter for in vitro transcription. To express anti-GPC3 scFv, multi-cloning sites (MCS) in the vector allowed insertion of a nucleic acid sequence comprising a Kozak sequence (SEQ ID NO: 59) operably linked to a nucleic acid sequence encoding a CD8α signal peptide (SEQ ID NO: 39) fused to the N-terminus of an anti-GPC3 scFv fragment into the CAR backbone vector, upstream and operably linked to the CAR backbone sequence. The nucleic acid sequence encoding the CD8α signal peptide and the anti-GPC3 scFv fragment was chemically synthesized and cloned into pLSINK-BBzBB CAR backbone via the EcoRI (5′-GAATTC-3′, SEQ ID NO:44) and SpeI (5′-ACTAGT-3′, SEQ ID NO:45) restriction sites by molecular cloning techniques known in the art. To co-express TGB23-6 protein, TGB23-6 sequence was also chemically synthesized and linked to the C-terminus of a CD3ζ intracellular signaling domain at cloning sites the HpaI (5′-GTTAAC-3′, SEQ ID NO:46) and MluI (5′-ACGCGT-3′, SEQ ID NO:47), which is separated by a peptide 2A linker (e.g., a P2A linker, SEQ ID NO: 48 or 60). The amino acid sequence of the anti-GPC3 CAR (named huLIC19309b CAR) described above is shown as SEQ ID NO: 36. The expressing sequence of the fusion polypeptide (named huLIC19309bT CAR) comprising anti-GPC3 CAR and TGB23-6 is shown as SEQ ID NO: 37 (amino acid sequence) and SEQ ID NO: 38 (nucleic acid sequence).

Lentiviral expression vectors expressing huLIC19309b CAR and huLIC19309bT CAR were prepared. The lentivirus packaging plasmid mixture containing pMDLg.pRRE, pRSV-REV and pMD2.G were pre-mixed with the vectors expressing CAR constructs at a pre-optimized ratio with polyetherimide, and then following the standard protocol as described in Example 1.

### 2. T Cell Purification and Activation

Human T cells were isolated from PBMCs (TPCS #A19Z284097) using Pan T cell isolation kit (Miltenyi #130-096-535), following manufacturer's protocol, see Example 1. The pooled enriched T cells were obtained and re-suspended in TexMACS™ GMP medium with 300 IU/mL IL-2.

The purified T cells were subsequently pre-activated for 48 hours with MACS GMP T cell TransAct™ kit (Miltenyi #170-076-156) according to manufacturer's protocol in which anti-CD3/CD28 MACSiBead particles were added at a bead-to-cell ratio of 40 μL/million.

### 3. CAR-T Preparation

The pre-activated T cells were re-suspended at 1×10⁶ cells in 24-well plates and then transduced with CAR-expressing lentivirus (multiplicity of infection=5) overnight. Then the transduced T cells were centrifuged at 300 g for 10 minutes and cultured with RPMI 1640 medium, 10% FBS and 300 IU/mL IL-2. Un-transduced T cells (UnT) were used as the negative control.

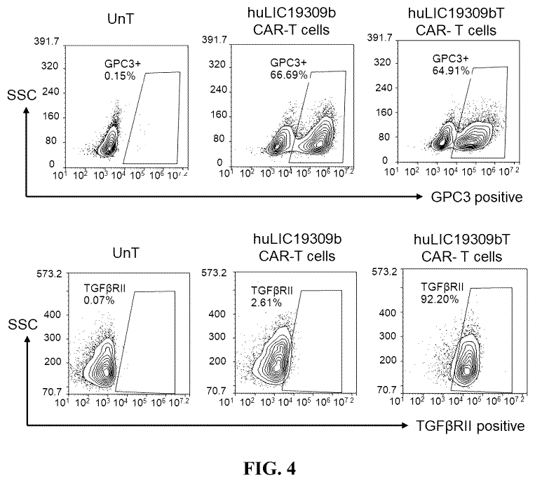

The cell surface expression of GPC3 CAR and TGFβRII were measured by FACS. The gates of GPC3 CAR and TGFβRII in CAR positive cells were bounded by cell population and TGFβRII isotype control, respectively. The CAR-T cells or UnT cells were re-suspended with 100 μL DPBS containing GPC3 protein-His tag (Acrobiosystems #GP3-H52H4) and incubated for 40 min at 4° C. Then the cells were washed with DPBS and re-suspended with 100 μL DPBS containing FITC-anti His tag (Genscript #A01620) and PE-anti TGFβRII antibody (Miltenyi #130-115-024), and incubated for 30 min at 4° C. As shown in FIG. 4, the CAR positive rates of huLIC19309b CAR-T cells and huLIC19309bT CAR-T cells were 66.69% and 64.91%. The expression levels of TGFβRII in CAR positive cells of UnT, huLIC19309b CAR-T cells and huLIC19309bT CAR-T cells were 0.07%, 2.61% and 92.20%, respectively.

### Example 3. The Expression Levels of pSmad2, pStat3 and pStat4 in CAR-T Cells Expressing TGB23 Treated with TGFβ1

TGFβ1 ligation to a tetrameric complex containing two units of TGFβR1 and TGFβR2 induces Smad2 phosphorylation to propagate an immunosuppressive signal to the cell nucleus (WO2018094244A1). Thus, phosphor-Smad2 (pSmad2) expression is used to interrogate TGFβ signaling pathway activation. The cellular response to IL-23 is initiated by receptor dimerization and phosphorylation of Stat3 and Stat4. Thus, pStat3 and pStat4 expression are used to assess IL-23 receptor signaling pathway activation referring to Robinson RT. IL12Rβ1: the cytokine receptor that we used to know. Cytokine. 2015 February; 71(2):348-59.

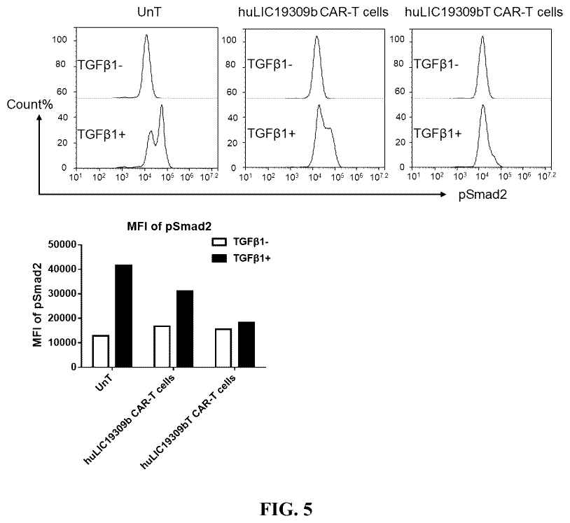

The CAR-T cells and UnT cells were treated with 20 ng/mL recombinant human TGFβ1 (R&D #240-B-002) for 4 hours, and then the cells were measured by FACS for pSmad2. The CAR-T cells or UnT cells were fixed with paraformaldehyde and permeabilized with Tween-20 for 10 minutes. The cells were re-suspended with 100 μL DPBS containing GPC3 protein-His tag and pSmad2 antibody (Cell signaling technology #18338) for 40 min at 4° C. The cells were washed with DPBS and re-suspended with 100 μL DPBS containing PE-anti His tag antibody (Biolegend #362603) and anti-rabbit IgG-Alexa Fluor 488 antibody (Thermo Fisher #A11034) for 30 min at 4° C., then detected by FACS. As shown in FIG. 5, after being treated with 20 ng/mL TGFβ1, the phosphorylation of Smad2 in UnT or huLIC19309b CAR-T cells obviously increased compared to that in huLIC19309bT CAR-T cells. The median fluorescence intensity (MFI) of pSmad2 in UnT, huLIC19309b CAR-T cells and huLIC19309bT CAR-T cells treated with 20 ng/mL TGFβ1 were 41565, 30972 and 18142, respectively. The data demonstrate that the expression of TGB23 constructs renders CAR-T cells insensitive to TGFβ immunosuppressive signaling.

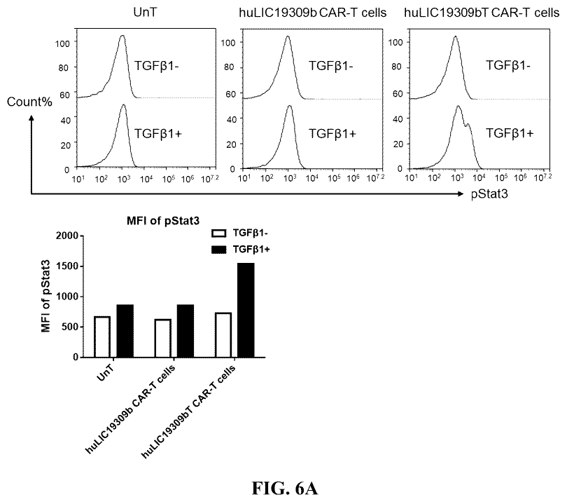

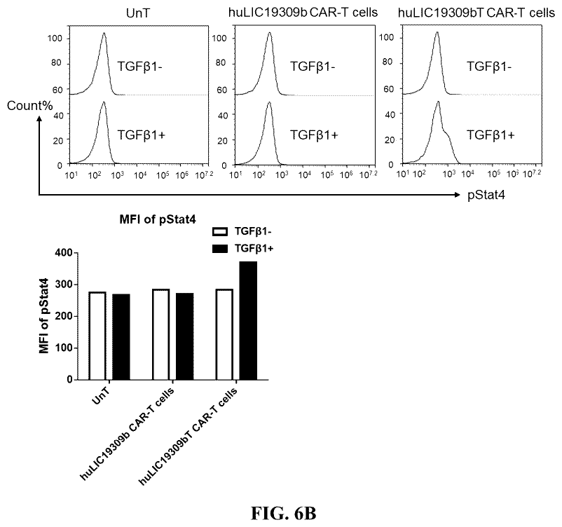

After being treated with 20 ng/mL TGFβ 1 for 4 hours, the cells were measured by FACS using pStat3 antibody (Biolegend #651004) and pStat4 antibody (Thermo Fisher #17-9044-42). As shown in FIG. 6A and FIG. 6B, the phosphorylation of Stat3 and Stat4 in huLIC19309bT CAR-T cells obviously increased after being treated with 20 ng/mL TGFβ 1 compared to that in UnT and huLIC19309b CAR-T cells. The MFI of pStat3 in UnT, huLIC19309b CAR-T cells and huLIC19309bT CAR-T cells treated with 20 ng/mL TGFβ1 were 854, 858 and 1543, respectively (FIG. 6A). The MFI of pStat4 in UnT, huLIC19309b CAR-T cells and huLIC19309bT CAR-T cells treated with 20 ng/mL TGFβ1 were 267, 271 and 370, respectively (FIG. 6B). The data validate that the converted TGFβ signal can induce IL-23 receptor signaling in CAR-T cells expressing TGB23 constructs.

These results show that huLIC19309bT CAR-T cells expressing anti-GPC3 CAR and TGB23 fusion protein could inhibit the phosphorylation of Smad2 and promote the phosphorylation of Stat3 and Stat4 under the stimulation of TGFβ1, which indicates that huLIC19309bT CAR-T cells can convert TGFβ signaling into intracellular IL-23 signaling and cause the response in downstream. To summarize, the results above firmly show that TGB23-6 construct converts a TGFβ mediated inhibitory signal to an IL23 mediated pro-proliferation signal in CAR-T cells, which grants CAR-T cells more potency to kill target cells (e.g., tumor cells) in the presence of TGFβ.

### Example 4. Cell Viability of CAR-T Cells Expressing TGB23-6 after being Treated with TGFβ1

3.5 million CAR positive cells were cultured in 6-well plates with RPMI 1640 medium, 10% FBS and 20 ng/mL TGFβ1 7 days after transduction. Cell viability and CAR positive cells were detected by cell counter and FACS every two days.

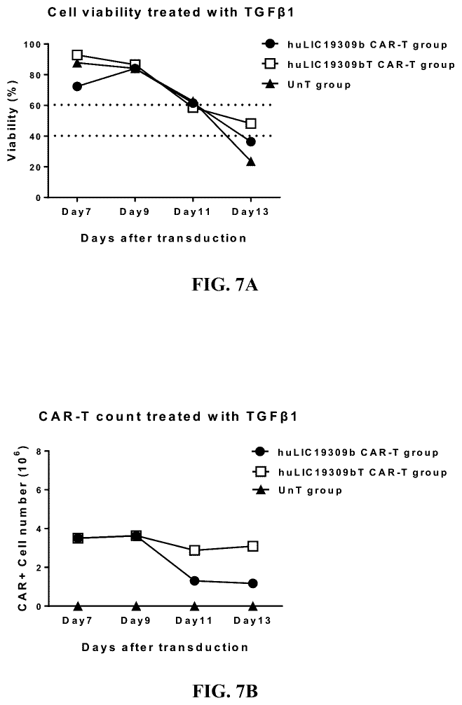

As shown in FIG. 7A and FIG. 7B, the cell viability and the number of CAR positive cells of UnT group and huLIC19309b CAR-T group decreased significantly compared with huLAb19309bT CAR-T group. The cell viability of UnT, huLIC19309b CAR-T cells and huLIC19309bT CAR-T cells were 23.6%, 36.4% and 48.2% on Day13 (FIG. 7A). CAR positive cells number of huLIC19309b CAR-T group showed a downward trend, while the cell number of huLIC19309bT CAR-T group was maintained (FIG. 7B). These results suggest that huLIC19309bT CAR-T cells expressing anti-GPC3 CAR and TGB23-6 structure can survive better than huLIC19309b CAR-T cells expressing anti-GPC3 CAR only in the presence of TGFβ1.

### Example 5. The Re-Challenge Model in CAR-T Cells Expressing TGB-23

### 1. The Re-Challenge Model Preparation

To evaluate the persistence and exhaustion of CAR-T cells in vitro, CAR-T cells repeat challenge model (“re-challenge model”) was set up. CAR-T cells were constantly stimulated by GPC3 positive tumor cells for several rounds to gain an exhaustion phenotype of CAR-T cells. There were two groups of treatment in re-challenge assay: huLIC19309b CAR-T group (the initial CAR-T cells were huLIC19309b CAR-T cells) and huLIC19309bT CAR-T group (the initial CAR-T cells were huLIC19309bT CAR-T cells). As the first round (Round1), the CAR-T cells were co-cultured with PLCPRF5 cells (ATCC #CRL-8024) expressing GPC3 at effector: target (E/T) ratio of 1:1 overnight in the presence of 5 ng/mL TGFβ1. The expressions of PD1 of CD4+CAR+ cells and of CD8+CAR+ cells were detected by FACS. Cells were re-suspended in fresh medium (RPMI 1640 medium, 10% FBS, 5 ng/mL TGFβ1 and 300 IU/mL IL2) after centrifuged at 300 g for 10 minutes and cultured for another two days. The viability, count, CAR positive ratio and killing potency of cells were analyzed. The cells were then reused for the same treatments as Round1, totally for another four rounds. The CAR-T cells without TGFβ1 treatment were used as control. PLCPRF5 cell number were adjusted at each round according to the number of CAR-T cells after each round of target cells stimulation.

### 2. The PD1 Expression in Re-Challenge Model

The PD1 expression in CAR-T cells after co-culture with PLCPRF5 cells overnight was detected by FACS. The cells collected from each stimulation round were re-suspended with 100 μL DPBS containing GPC3 protein-His tag (Acrobiosystems #GP3-H52H4) and incubated for 40 min at 4° C. Then the cells were washed with DPBS and re-suspended with 100 μL DPBS containing FITC-anti His tag antibody (Genscript #A01620), CD4 antibody (Biolegend #357410), CD8 antibody (Biolegend #344710) and PD1 antibody (Mitenyi #130-117-694), and incubated for 30 min at 4° C.

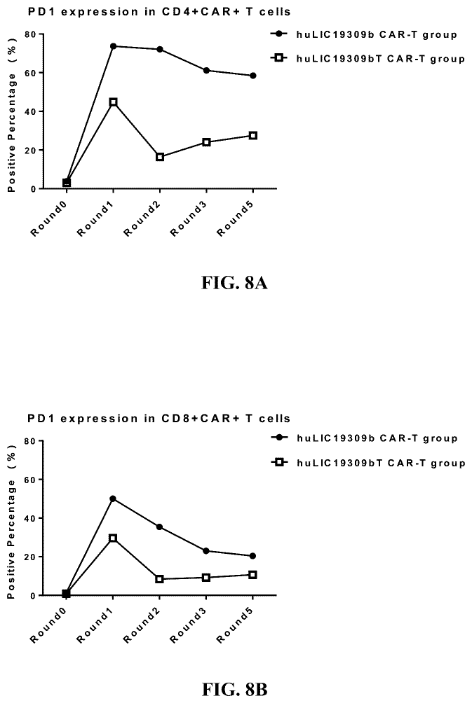

The levels of PD1 expressed in CAR positive subset of huLIC19309b CAR-T group increased significantly from 3.9% to 73.7%, 72.1%, 61.1% and 58.5% (CD4+CAR+ T cells), and from 1.4% to 50.0%, 35.4%, 23.0% and 20.4% (CD8+CAR+ T cells) (FIG. 8) during the 5 rounds experiments. In contrast, the PD1 expression in CAR positive subset of huLIC19309bT CAR-T group increased to 27.5% (CD4+CAR+ T cells) and increased to 10.7% (CD8+CAR+ T cells) at Round 5 (FIGS. 8A and 8B). PD1 expression of huLIC19309bT CAR-T cells was lower than that of huLIC19309b CAR-T cells in re-challenge model, indicating that less TGB23 CAR-T cells were exhausted as compared to CAR-T cells not transduced with TGB23.

### 3. The Percentage of CAR Positive T Cells in Re-Challenge Mode

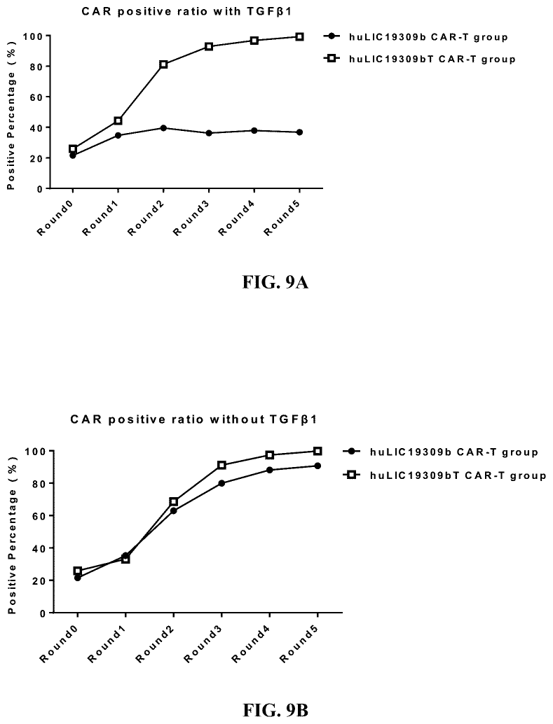

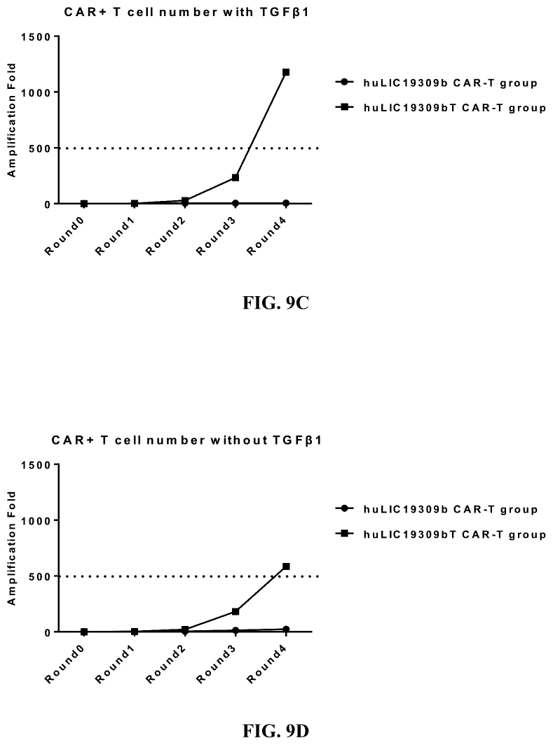

The percentage of CAR positive T cells after co-culture with PLCPRF5 cells was detected by FACS at the end of each stimulation round. The results are shown in FIGS. 9A and 9B. In the presence of 5 ng/mL TGFβ1, the percentage of CAR positive T cells of huLIC19309b CAR-T group increased from 21.6% to 36.8% during the re-challenge model assay. In comparison, the percentage of CAR positive T cells of huLIC19309bT CAR-T group increased from 25.9% to 99.3%. In the absence of TGFβ 1, the percentage of CAR positive T cells increased almost similarly between the two groups, specifically, from 21.6% to 90.7% (huLIC19309b CAR-T group) and from 25.9% to 99.8% (huLIC19309bT CAR-T group) respectively. These results indicate that TGFβ impeded the proliferative potency of CAR-T on killing target cells, while co-expression of TGB23-6 construct in CAR-T cells helped CAR-T cells to conquer the inhibitory function of TGFβ and promote expansion of the CAR-T while killing target cells.

The counts of CAR-T cells after co-culture with PLCPRF5 cells were detected by T4 Cell Counter with Trypan blue staining at end of each round. The 20 μL cells suspension was mixed with 20 μL Trypan blue, pipetted into the disposable counting chamber and analyzed by Cellometer T4. The amplification fold of CAR-T cells was calculated according to the total number of T cells and the percentage of CAR positive cells.

As shown in FIG. 9C and FIG. 9D, the amplification fold of huLIC19309b CAR-T cells increased from 1 to 5.8 folds in the presence of 5 ng/mL TGFβ1 and from 1 to 23 folds in the absence of TGFβ1. On the contrary, the amplification fold of huLIC19309bT CAR-T cells increased from 1 to 1177 folds in the presence of 5 ng/mL TGFβ1 and from 1 to 586 folds in the absence of TGFβ1. These results indicate that co-expression of TGB23-6 in CAR-T cells not only conquered the TGFβ inhibitory effect, but also further promoted the proliferation of the CAR-T cells in the presence of TGFβ and serial stimulation of antigen.

### 4. The Cytotoxicity Assay in Re-Challenge Model

The cytotoxicity of CAR-T cells was assessed at the end of each rounds in CAR-T re-challenge assay at total T cells: target cells ratio of 1:1 by One-glo luminescent luciferase assay. GPC3 positive human hepatocellular carcinoma (HCC) cell lines Hep3B2.1-7.Luc (ATCC #HB-8064) and PLCPRF5-Luc (ATCC #CRL-8024) were used as target cells. The target cells (2×10³/well each) were seeded in 384-well plates and then incubated with the cells collected at the end of each rounds at the indicated E/T ratio for 24 hours. Then the One-glo luminescent luciferase assay reagents (Promega #E6110) were added to each well and remaining luminescence was detected with microplate reader. Since luciferase is expressed only in tumor cells, the remaining luminescence correlates directly to the number of viable target cells in the well. The maximum and minimum luciferase activity were obtained by adding UnT cells and medium, respectively. The specific cytotoxicity was calculated by the formula: Specific Cytotoxicity %=100%×(1−(RLUsample−RLUmin)/(RLUmax−RLUmin).

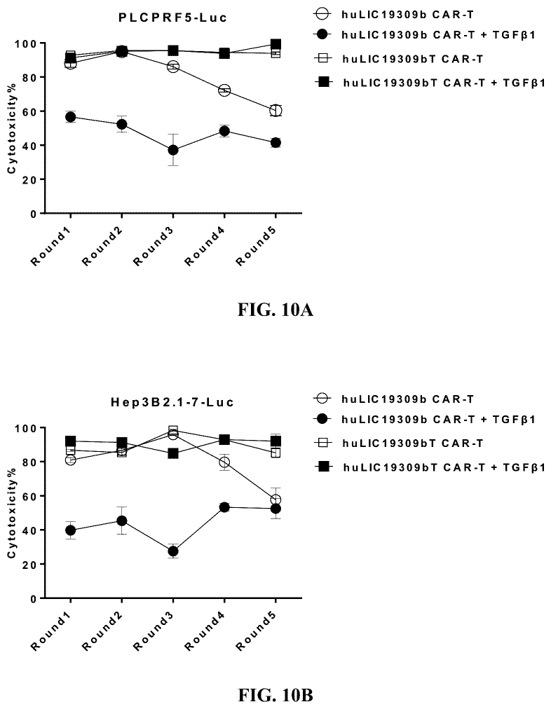

As shown in FIG. 10A, huLIC19309b CAR-T group treated without TGFβ1 in re-challenge model showed decreased cytotoxicity on PLCPRF5-Luc at the 1:1 ratio (from 88.1%±2.4% to 60.4%±3.1%), while in the presence of 5 ng/mL TGFβ1, the cytotoxicity potency decreased further, from 56.6%±3.3% to 41.5%±2.6%. In comparison, the cytotoxicity of huLIC19309bT CAR-T group on PLCPRF5.Luc cells exceeded 80% during the experiment, regardless of TGFβ1. The huLIC19309bT CAR-T group also showed higher cytotoxicity on PLCPRF5.Luc cells (from 92.8%±0.5% to 94.0%±0.7% without TGFβ1 and from 91.2%±2.1% to 99.4%±1.2% with TGFβ1) from Round1 to Round5. The similar tendency was repeated using another target cells (Hep3B2.1-7-Luc cells, FIG. 10B). These results indicate that CAR-T cells (huLIC19309bT) co-expressing TGB23-6 construct obtained not only resistance against TGFβ1 mediated inhibitory signaling, but also better persistence against stimulation induced senescence.

### Example 6. The Real Time Cellular Analysis (RTCA) Assay in CAR-T Cells Expressing TGB23-6

The real time cytotoxicity potency in CAR-T cells co-cultured with Hep3B2.1-7 cells was measured by RTCA assay. The Hep3B2.1-7 cells (4×10³/well each) were seeded in 96-well plates and detected by RTCA analyzer overnight. Then the target cells were incubated with CAR-T or UnT cells at the E/T ratio of 1:20, during which the cytotoxicity potency of T cells was detected in real time. The cell index indicates the activity, adhesion and number of the target cells.

As shown in FIG. 11, the cell index of Hep3B2.1-7 cells co-cultured with UnT cells increased over time significantly and the cell index at the end of the experiment was 13.8. The CAR-T cells (huLIC19309b CAR-T cells and huLIC19309bT CAR-T cells) showed cytotoxicity on Hep3B2.1-7 cells at different extent with the extension of time. The cell indexes of Hep3B2.1-7 cells co-cultured with huLIC19309b CAR-T cells and huLIC19309bT CAR-T cells were 8.3 and 2.1 at the end, respectively. These results suggest that huLIC19309bT CAR-T cells co-expressing TGB23-6 construct inhibited the Hep3B2.1-7 cells proliferation more efficiently than expressing CAR alone, especially during later co-culture stage. The results demonstrate CAR-T cells transduced with TGB23-6 exhibited durable cytotoxicity against target cells.

### Example 7. The Anti-Tumor Effect of CAR-T Cells Expressing TGB-23 In Vivo

In vivo anti-tumor efficacy of CAR-T cells was evaluated in NCG mouse xenograft model. For the subcutaneous xenograft model, NCG mice aged 6-7 weeks were inoculated with Hep3B2.1-7 cells subcutaneously in the right lower back (3.7×10⁶ cells/mouse). When the average tumor volume approached to 150 mm³ (day 6 after the xenograft inoculation), mice were randomized into 5 groups and treated with huLIC19309b CAR-T cells (0.3 M or 0.8 M cells/mouse), huLIC19309bT CAR-T cells (0.3 M or 0.8 M cells/mouse) and UnT cells (0.8 M cells/mouse), respectively, by tail vein injection. Tumor size was measured with digital calipers twice per week. Mice would be sacrificed early when the tumor volume reached more than 2000 mm³. Tumor volume was calculated according to the following formula:

$$Tumor volume = \frac{( length ) \times ( width ) 2}{2} .$$

The amplification of T cells in mouse blood was also detected by FACS on the day 7, 14, 21 and 28 after the adoptive transfer, respectively.

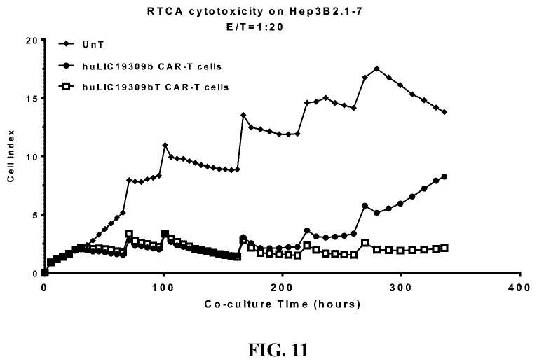

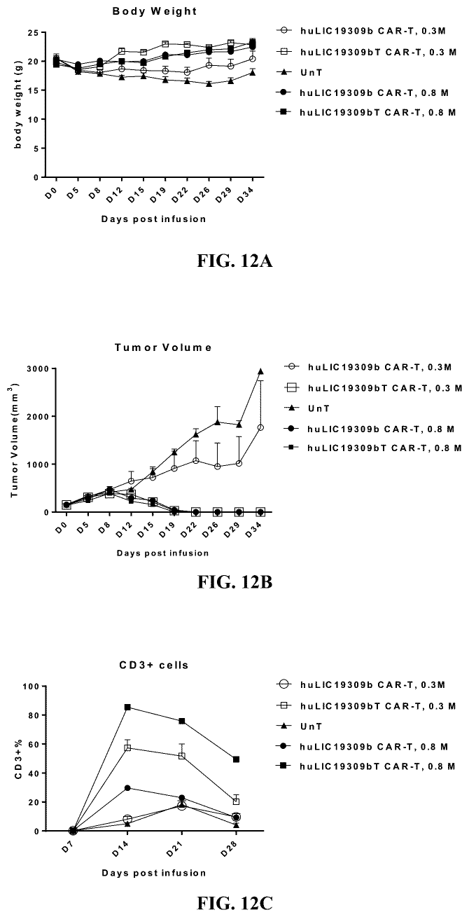

As shown in FIG. 12A, the body weights of these mice were measured twice per week and no mouse bodyweight loss was observed in all groups whether 0.3 M dosage or 0.8 M dosage during this experiment except UnT group. The body weights in UnT group decreased 12.2% (18.0 g at day 34 vs 20.5 g at day 0) on day 34, due to the heavy tumor burden.

As shown in FIG. 12B, at the dosage of 0.3 million (M)/mouse, compared to the UnT group, huLAb19309b CAR-T group showed a 27% tumor reduction in tumor size (911.4 mm³ vs 1248.5 mm³) while the mice administered with huLAb19309bT CAR-T cells exhibited about 97% reduction in tumor size (29.0 mm³ vs 1248.5 mm³) on day 19. After day 19, the mice treated with huLIC19309bT CAR-T cells had almost tumor free, which suggested huLIC19309bT CAR-T cells had better therapeutic potential than huLIC19309b CAR-T cells at lower dosage (0.3 M/mouse). When the dosage was increased to 0.8 M/mouse, mice administered with huLIC19309bT CAR-T cells exhibited an obvious inhibitory effect on tumor growth with a growth inhibition rate of 81.4% in comparison with UnT (156.6 mm³ vs 843.8 mm³) on day 15, and similar tumor regression were observed in the huLIC19309b CAR-T cells treatment group (244.1 mm³ vs 843.8 mm³). The mice treated with huLIC19309bT CAR-T cells and huLIC19309b CAR-T cells had almost tumor free on day 19 and day 22, respectively.

The amplification of T cells in mouse blood was also detected by FACS on the day 7, 14, 21 and 28 after the adoptive transfer. The mouse blood was centrifuged at 500 g for 10 min to separate plasma and blood cells, then the blood cells were incubated with red blood cell lysis buffer for 10 min. After lysis, the cells were washed with DPBS and stained with CD3 antibody (Biolegend #300316). As shown in FIG. 12C, T cells of the mice in huLIC19309bT CAR-T cells group exhibited significant expansion on day 14 (57.4% CD3 positive cells) and reduced subsequently at 0.3 M dosage, while there were no obvious expansions in the mice treated with huLIC19309b CAR-T cells (8.1% CD3 positive cells) and UnT (5.1% CD3 positive cells) on day 14. In addition, huLIC19309b CAR-T cells and huLIC19309bT CAR-T cells showed similar anti-tumor potency at 0.8 M dosage, but huLIC19309bT CAR-T cells exhibited a better expansion than huLIC19309b CAR-T cells (85.5% CD3 positive cells vs. 29.7% CD3 positive cells) on day 14.

### Example 8. Generation of LCAR-UL186S T Cells Expressing TGB23

### 1. Construction of LCAR-UL186S and TGB23-6 Expression Vectors

pLVX-Puro vector purchased from Clontech was digested with ClaI and EcoRI restriction enzymes, and the original CMV promoter was replaced with a human EF1α promoter (GenBank: J04617.1) to obtain the pLVX-hEF1α vector. Gene of LCAR-UL186S (SIV Nef_M116-IRES-CD8α SP-CD20 scFv (Leu16)-CD8α Hinge-CD8α TM-4-1BB-ITAM010, SEQ ID NO: 90) disclosed in PCT patent applications No. PCT/CN2020/112181 and PCT/CN2020/112182 was cloned into pLVX-hEF1α to form a recombinant CD20 CAR (SEQ ID NO: 89) expression plasmid, named as M1439. Gene of TGB23-6 was cloned into pLVX-hEF1α to form a recombinant TGB23-6 expression plasmid, named as M1647. The recombinant expression vectors M1439 and M1647 were individually mixed with psPAX2 and pMD2.G helper plasmids in a certain proportion, and co-transfected into HEK 293T cells. 60 hours after transfection, the cell culture supernatant containing the viral vector was collected and centrifuged at 4° C. and 3000 g for 5 min. After the supernatant was filtered through a 0.45 μm filter, the 500 KD hollow fiber membrane column tangential flow technique was used to further concentrate to prepare a lentivirus concentrate, which was stored at −80° C. for later use.

### 2. Preparation of LCAR-UL186S T Cells and LCAR-UL186S+TGB23-6 T Cells

T lymphocytes were purified from peripheral blood mononuclear cells (PBMC) purchased from TPCS company, using Pan T Cell Isolation Kit (Miltenyi Biotech). The purified T cells were activated by CD3/CD28 magnetic beads, and then cultured in a 37° C., 5% CO₂ incubator for 24 hours. Subsequently, T cells were transduced with above mentioned virus vectors to produce LCAR-UL186S T cells (transduced with M1439 vector) or LCAR-UL186S+TGB23-6 T cells (transduced with M1439 and M1647 vectors). After expansion in several days, TCRαβ negative LCAR-UL186S T cells and LCAR-UL186S+TGB23-6 T cells were enrichened using TCRαβ sorting kit (TCRα/β-Biotin, CliniMACS, 6190221004; Anti-Biotin Reagent, CliniMACS, 6190312010). 5×10⁵ cell suspensions each from final products were aspirated, centrifuged at room temperature to discard the supernatant, and then resuspended in DPBS. 1 μL Anti-human CD5-PE-CY7 (Biolegend, Cat. No. 364008), 1 μL Alex Fluor 488 Anti-CAR (LUCAR)-20S (antibody against CD20 CAR produced by Genscript, order No. LGBUADAb-1), 1 μL APC anti-human TCR α/β antibody (Biolegend, Cat. No.: B259839), and 1 μL PE anti-human TGF-β RII Antibody (Miltenyi Biotech, Cat. No.: 130115024) were added to the cells, and incubated at 4° C. for 30 min. The cells were resuspended twice with 1 mL DPBS, centrifuged at room temperature to discard the supernatant, and finally the cells were resuspended in DPBS for flow cytometry to detect the positive rates of CD20 CAR, TCR αβ, and TGB23-6.

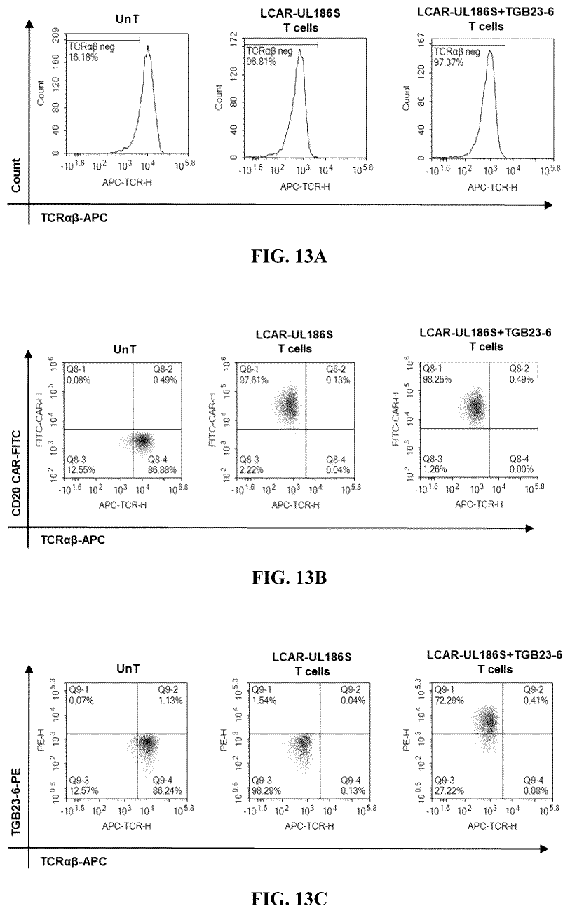

As shown in FIGS. 13A-13C, TCRαβ negative ratios of LCAR-UL186S T cells and LCAR-UL186S+TGB23-6 T cells after sorting and enrichment are 96.81% and 97.37%, respectively. Both TCRαβ negative and CD20 CAR positive ratios of LCAR-UL186S T cells was 97.61% and that of LCAR-UL186S+TGB23-6 T cells was 98.25%. Both TCRαβ negative and TGB23-6 positive ratios of LCAR-UL186S T cells was 1.54% and that of LCAR-UL186S+TGB23-6 T cells was 72.29%, indicating the successful expression of TGB23-6. Un-transduced T cells (UnT) were used as a negative control.

### Example 9. Specific Killing Activity of LCAR-UL186S T Cells Expressing TGB23

To further study the specific killing ability of LCAR-UL186S T cells and LCAR-UL186S+TGB23-6 T cells to CD20 positive cells (lymphoma cells Raji). LCAR-UL186S T cells and LCAR-UL186S+TGB23-6 T cells were mixed individually with Raji cells in Corning 384 well plates at effector-target ratio of 20:1, 10:1, or 5:1, and incubated for 12 h-18 h. 25 μL of One-Glo reagent (TAKARA, Cat. No. B6120) was added to each well, and then the fluorescence value of Luciferase was detected by a microplate reader (TECAN, spark 10M). The cytotoxic effect of T lymphocytes against target cells in each group was calculated by the formula: Killing efficiency=1−[(detection sample value-min value)/max value].

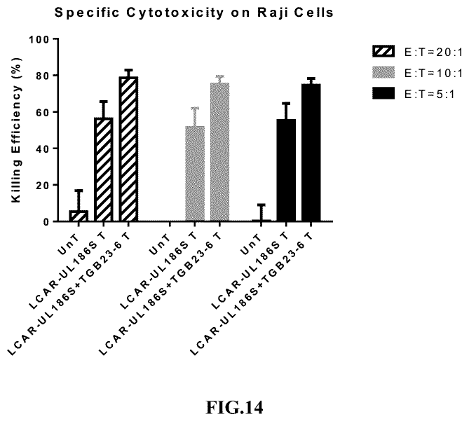

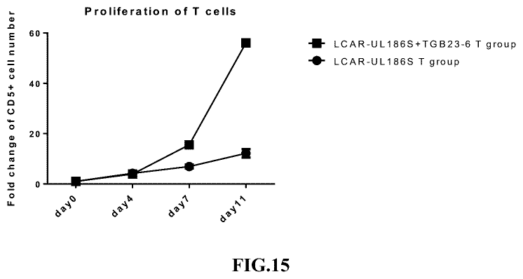

As shown in FIG. 14, at the ratios of effector-targets of 20:1, 10:1 and 5:1, compared with UnT cells, LCAR-UL186S T cells and LCAR-UL186S+TGB23-6 T cells could effectively kill target cells Raji (P<0.05). There is no significant difference in cytotoxicity between LCAR-UL186S T and LCAR-UL186S+TGB23-6 T groups (P>0.05), suggesting that in vitro TGB23-6 dose not affect the specificity of LCAR-UL186S to target cells.

### Example 10. Proliferation of LCAR-UL186S T Cells Expressing TGB23 after Multiple Rounds of Target Cell Stimulation

As mentioned above, LCAR-UL186S T cells and LCAR-UL186S+TGB23-6 T cells were produced. The cell density of Raji was adjusted to 1×10⁶ cells/mL. Raji cells were treated with 20 g/mL mitomycin at 37° C. for 3 h, washed three times with 10 mL DPBS, and resuspended in the culture medium. The treated Raji cells were mixed and co-cultured with LCAR-UL186S T cells or LCAR-UL186S+TGB23-6 T cells at a ratio of 1:1. LCAR-UL186S T cells and LCAR-UL186S+TGB23-6 T cells were stimulated repeatedly with Raji at the E/T ratio of 1:1 on day4 and day7 after the first stimulation. The cells were harvested at day0, day4, day7 and day11. Cell numbers were counted with K2 automated cell counter counting, and the expression of CD5 was detected by flow cytometry. T lymphocytes counts were calculated via multiplying total cell number by positive ratio of CD5 and the proliferation curves with the fold change of CD5+ cell number based on day 0 were drawn.

As shown in FIG. 15, after three rounds of stimulation of Raji cells in vitro, LCAR-UL186S T cells expanded about 12 folds, while LCAR-UL186S+TGB23-6 T cells expanded about 56 folds. LCAR-UL186S+TGB23-6 T cells expanded over 4 times more than LCAR-UL186S T cells (P<0.05), suggesting that TGB23-6 is able to significantly improve the proliferation ability of LCAR-UL186S T cells in vitro.

### SEQUENCE LISTING

SEQ ID NO: 1. signal peptide of TGFβR1
MEAAVAAPRPRLLLLVLAAAAAAAAALLPGATA
SEQ ID NO: 2. signal peptide of TGFβR2
MGRGLLRGLWPLHIVLWTRIAS
SEQ ID NO: 3. extracellular domain of TGFβR1
LQCFCHLCTKDNFTCVTDGLCFVSVTETTDKVIHNSMCIAEIDLIPRDRPFVCAPSSKTGS
VTTTYCCNQDHCNKIELPTTVKSSPGLGPVEL
SEQ ID NO: 4. extracellular domain of TGFβR2
TIPPHVQKSVNNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGETFFMCSCSSD
ECNDNIIFSEEYNTSNPDLLLVIFQ
SEQ ID NO: 5. transmembrane domain of TGFβR1
AAVIAGPVCFVCISLMLMVYI
SEQ ID NO: 6. transmembrane domain of TGFβR2
VTGISLLPPLGVAISVIIIFY
SEQ ID NO: 7. transmembrane domain of IL-23R
LLLGMIVFAVMLSILSLIGIF
SEQ ID NO: 8. transmembrane domain of IL-12Rβ1
WLIFFASLGSFLSILLVGVLGYLGL
SEQ ID NO: 9. The sequence of TGFβR1 intracellular domain near the membrane
CHNRTV
SEQ ID NO: 10. The sequence of TGFβR2 intracellular domain near the membrane
CYRVNRQ
SEQ ID NO: 11. The sequence of IL-23R extracellular domain near the membrane
VPQVTSKAFQHDTWNSGLTVASISTGHLTSDNRGDIG
SEQ ID NO: 12. The sequence of IL-12Rβ1 extracellular domain near the membrane
VSD
SEQ ID NO: 13. intracellular domain of TGFβR1
CHNRTVIHHRVPNEEDPSLDRPFISEGTTLKDLIYDMTTSGSGSGLPLLVQRTIARTIVLQ
ESIGKGRFGEVWRGKWRGEEVAVKIFSSREERSWFREAEIYQTVMLRHENILGFIAADN
KDNGTWTQLWLVSDYHEHGSLFDYLNRYTVTVEGMIKLALSTASGLAHLHMEIVGTQ
GKPAIAHRDLKSKNILVKKNGTCCIADLGLAVRHDSATDTIDIAPNHRVGTKRYMAPEV
LDDSINMKHFESFKRADIYAMGLVFWEIARRCSIGGIHEDYQLPYYDLVPSDPSVEEMR
KVVCEQKLRPNIPNRWQSCEALRVMAKIMRECWYANGAARLTALRIKKTLSQLSQQEG
IKM
SEQ ID NO: 14. intracellular domain of TGFβR2
CYRVNRQQKLSSTWETGKTRKLMEFSEHCAIILEDDRSDISSTCANNINHNTELLPIELDT
LVGKGRFAEVYKAKLKQNTSEQFETVAVKIFPYEEYASWKTEKDIFSDINLKHENILQFL
TAEERKTELGKQYWLITAFHAKGNLQEYLTRHVISWEDLRKLGSSLARGIAHLHSDHTP
CGRPKMPIVHRDLKSSNILVKNDLTCCLCDFGLSLRLDPTLSVDDLANSGQVGTARYMA
PEVLESRMNLENVESFKQTDVYSMALVLWEMTSRCNAVGEVKDYEPPFGSKVREHPCV
ESMKDNVLRDRGRPEIPSFWLNHQGIQMVCETLTECWDHDPEARLTAQCVAERFSELE
HLDRLSGRSCSEEKIPEDGSLNTTK
SEQ ID NO: 15. intracellular domain of IL-23R
NRSFRTGIKRRILLLIPKWLYEDIPNMKNSNVVKMLQENSELMNNNSSEQVLYVDPMIT
EIKEIFIPEHKPTDYKKENTGPLETRDYPQNSLFDNTTVVYIPDLNTGYKPQISNFLPEGSH
LSNNNEITSLTLKPPVDSLDSGNNPRLQKHPNFAFSVSSVNSLSNTIFLGELSLILNQGECS
SPDIQNSVEEETTMLLENDSPSETIPEQTLLPDEFVSCLGIVNEELPSINTYFPQNILESHEN
RISLLEK
SEQ ID NO: 16. intracellular domain of IL-12Rβ1
NRAARHLCPPLPTPCASSAIEFPGGKETWQWINPVDFQEEASLQEALVVEMSWDKGERT
EPLEKTELPEGAPELALDTELSLEDGDRCKAKM
SEQ ID NO: 17. mutant of IL-23R intracellular domain
NRSFRTGIKRRILLLIPKWLFEDIPNMKNSNVVKMLQENSELMNNNSSEQVLFVDPMITE
IKEIFIPEHKPTDFKKENTGPLETRDFPQNSLFDNTTVVFIPDLNTGFKPQISNFLPEGSHLS
NNNEITSLTLKPPVDSLDSGNNPRLQKHPNFAFSVSSVNSLSNTIFLGELSLILNQGECSSP
DIQNSVEEETTMLLENDSPSETIPEQTLLPDEFVSCLGIVNEELPSINTFFPQNILESHENRIS
LLEK
SEQ ID NO: 18. truncated intracellular domain of IL-23R
NRSFRTGIKRR
SEQ ID NO: 19. truncated intracellular domain of IL-12Rβ1
NRAAR
SEQ ID NO: 20. TGB23-1 amino acid
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELAAVIAGPVCFVCISLMLMVYICHNRTVNRSFRTGIKRRILLLIPKWLYEDIPNM
KNSNVVKMLQENSELMNNNSSEQVLYVDPMITEIKEIFIPEHKPTDYKKENTGPLETRDY
PQNSLFDNTTVVYIPDLNTGYKPQISNFLPEGSHLSNNNEITSLTLKPPVDSLDSGNNPRL
QKHPNFAFSVSSVNSLSNTIFLGELSLILNQGECSSPDIQNSVEEETTMLLENDSPSETIPEQ
TLLPDEFVSCLGIVNEELPSINTYFPQNILESHENRISLLEKATNFSLLKQAGDVEENPGPM
GRGLLRGLWPLHIVLWTRIASTIPPHVQKSVNNDMIVTDNNGAVKFPQLCKFCDVRFST
CDNQKSCMSNCSITSICEKPQEVCVAVWRKNDENITLETVCHDPKLPYHDFILEDAASPK
CIMKEKKKPGETFFMCSCSSDECNDNIIFSEEYNTSNPDLLLVIFQVTGISLLPPLGVAISVI
IIFYCYRVNRQNRAARHLCPPLPTPCASSAIEFPGGKETWQWINPVDFQEEASLQEALVV
EMSWDKGERTEPLEKTELPEGAPELALDTELSLEDGDRCKAKM
SEQ ID NO: 21. TGB23-2 amino acid
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVTGISLLPPLGVAIS
VIIIFYCYRVNRQNRSFRTGIKRRILLLIPKWLYEDIPNMKNSNVVKMLQENSELMNNNS
SEQVLYVDPMITEIKEIFIPEHKPTDYKKENTGPLETRDYPQNSLFDNTTVVYIPDLNTGY
KPQISNFLPEGSHLSNNNEITSLTLKPPVDSLDSGNNPRLQKHPNFAFSVSSVNSLSNTIFL
GELSLILNQGECSSPDIQNSVEEETTMLLENDSPSETIPEQTLLPDEFVSCLGIVNEELPSIN
TYFPQNILESHENRISLLEKATNFSLLKQAGDVEENPGPMEAAVAAPRPRLLLLVLAAAA
AAAAALLPGATALQCFCHLCTKDNFTCVTDGLCFVSVTETTDKVIHNSMCIAEIDLIPRD
RPFVCAPSSKTGSVTTTYCCNQDHCNKIELPTTVKSSPGLGPVELAAVIAGPVCFVCISL
MLMVYICHNRTVNRAARHLCPPLPTPCASSAIEFPGGKETWQWINPVDFQEEASLQEAL
VVEMSWDKGERTEPLEKTELPEGAPELALDTELSLEDGDRCKAKM
SEQ ID NO: 22. TGB23-3 amino acid
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELAAVIAGPVCFVCISLMLMVYICHNRTVNRAARHLCPPLPTPCASSAIEFPGGK
ETWQWINPVDFQEEASLQEALVVEMSWDKGERTEPLEKTELPEGAPELALDTELSLEDG
DRCKAKMATNFSLLKQAGDVEENPGPMGRGLLRGLWPLHIVLWTRIASTIPPHVQKSV
NNDMIVTDNNGAVKFPQLCKFCDVRFSTCDNQKSCMSNCSITSICEKPQEVCVAVWRK
NDENITLETVCHDPKLPYHDFILEDAASPKCIMKEKKKPGETFFMCSCSSDECNDNIIFSE
EYNTSNPDLLLVIFQVTGISLLPPLGVAISVIIIFYCYRVNRQNRSFRTGIKRRILLLIPKWL
YEDIPNMKNSNVVKMLQENSELMNNNSSEQVLYVDPMITEIKEIFIPEHKPTDYKKENTG
PLETRDYPQNSLFDNTTVVYIPDLNTGYKPQISNFLPEGSHLSNNNEITSLTLKPPVDSLD
SGNNPRLQKHPNFAFSVSSVNSLSNTIFLGELSLILNQGECSSPDIQNSVEEETTMLLENDS
PSETIPEQTLLPDEFVSCLGIVNEELPSINTYFPQNILESHENRISLLEK
SEQ ID NO: 23. TGB23-4 amino acid
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELVPQVTSKAFQHDTWNSGLTVASISTGHLTSDNRGDIGLLLGMIVFAVMLSILS
LIGIFNRSFRTGIKRRILLLIPKWLYEDIPNMKNSNVVKMLQENSELMNNNSSEQVLYVD
PMITEIKEIFIPEHKPTDYKKENTGPLETRDYPQNSLFDNTTVVYIPDLNTGYKPQISNFLP
EGSHLSNNNEITSLTLKPPVDSLDSGNNPRLQKHPNFAFSVSSVNSLSNTIFLGELSLILNQ
GECSSPDIQNSVEEETTMLLENDSPSETIPEQTLLPDEFVSCLGIVNEELPSINTYFPQNILE
SHFNRISLLEKATNFSLLKQAGDVEENPGPMGRGLLRGLWPLHIVLWTRIASTIPPHVQK
SVNNDMIVTDNNGAVKFPQLCKFCDVRFSTCDNQKSCMSNCSITSICEKPQEVCVAVW
RKNDENITLETVCHDPKLPYHDFILEDAASPKCIMKEKKKPGETFFMCSCSSDECNDNIIF
SEEYNTSNPDLLLVIFQVSDWLIFFASLGSFLSILLVGVLGYLGLNRAARHLCPPLPTPCA
SSAIEFPGGKETWQWINPVDFQEEASLQEALVVEMSWDKGERTEPLEKTELPEGAPELA
LDTELSLEDGDRCKAKM
SEQ ID NO: 24. TGB23-5 amino acid
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVPQVTSKAFQHDT
WNSGLTVASISTGHLTSDNRGDIGLLLGMIVFAVMLSILSLIGIFNRSFRTGIKRRILLLIPK
WLYEDIPNMKNSNVVKMLQENSELMNNNSSEQVLYVDPMITEIKEIFIPEHKPTDYKKE
NTGPLETRDYPQNSLFDNTTVVYIPDLNTGYKPQISNFLPEGSHLSNNNEITSLTLKPPVD
SLDSGNNPRLQKHPNFAFSVSSVNSLSNTIFLGELSLILNQGECSSPDIQNSVEEETTMLLE
NDSPSETIPEQTLLPDEFVSCLGIVNEELPSINTYFPQNILESHENRISLLEKATNFSLLKQA
GDVEENPGPMEAAVAAPRPRLLLLVLAAAAAAAAALLPGATALQCFCHLCTKDNFTCV
TDGLCFVSVTETTDKVIHNSMCIAEIDLIPRDRPFVCAPSSKTGSVTTTYCCNQDHCNKIE
LPTTVKSSPGLGPVELVSDWLIFFASLGSFLSILLVGVLGYLGLNRAARHLCPPLPTPCAS
SAIEFPGGKETWQWINPVDFQEEASLQEALVVEMSWDKGERTEPLEKTELPEGAPELAL
DTELSLEDGDRCKAKM
SEQ ID NO: 25. TGB23-6 amino acid
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELVSDWLIFFASLGSFLSILLVGVLGYLGLNRAARHLCPPLPTPCASSAIEFPGGK
ETWQWINPVDFQEEASLQEALVVEMSWDKGERTEPLEKTELPEGAPELALDTELSLEDG
DRCKAKMATNFSLLKQAGDVEENPGPMGRGLLRGLWPLHIVLWTRIASTIPPHVQKSV
NNDMIVTDNNGAVKFPQLCKFCDVRFSTCDNQKSCMSNCSITSICEKPQEVCVAVWRK
NDENITLETVCHDPKLPYHDFILEDAASPKCIMKEKKKPGETFFMCSCSSDECNDNIIFSE
EYNTSNPDLLLVIFQVPQVTSKAFQHDTWNSGLTVASISTGHLTSDNRGDIGLLLGMIVF
AVMLSILSLIGIFNRSFRTGIKRRILLLIPKWLYEDIPNMKNSNVVKMLQENSELMNNNSS
EQVLYVDPMITEIKEIFIPEHKPTDYKKENTGPLETRDYPQNSLFDNTTVVYIPDLNTGYK
PQISNFLPEGSHLSNNNEITSLTLKPPVDSLDSGNNPRLQKHPNFAFSVSSVNSLSNTIFLG
ELSLILNQGECSSPDIQNSVEEETTMLLENDSPSETIPEQTLLPDEFVSCLGIVNEELPSINT
YFPQNILESHFNRISLLEK
SEQ ID NO: 26. TGB23-14 amino acid
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELVPQVTSKAFQHDTWNSGLTVASISTGHLTSDNRGDIGLLLGMIVFAVMLSILS
LIGIFNRSFRTGIKRRILLLIPKWLFEDIPNMKNSNVVKMLQENSELMNNNSSEQVLFVDP
MITEIKEIFIPEHKPTDFKKENTGPLETRDFPQNSLFDNTTVVFIPDLNTGFKPQISNFLPEG
SHLSNNNEITSLTLKPPVDSLDSGNNPRLQKHPNFAFSVSSVNSLSNTIFLGELSLILNQGE
CSSPDIQNSVEEETTMLLENDSPSETIPEQTLLPDEFVSCLGIVNEELPSINTFFPQNILESHF
NRISLLEKATNFSLLKQAGDVEENPGPMGRGLLRGLWPLHIVLWTRIASTIPPHVQKSVN
NDMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSEE
YNTSNPDLLLVIFQVSDWLIFFASLGSFLSILLVGVLGYLGLNRAARHLCPPLPTPCASSAI
EFPGGKETWQWINPVDFQEEASLQEALVVEMSWDKGERTEPLEKTELPEGAPELALDTE
LSLEDGDRCKAKM
SEQ ID NO: 27. TGB23-15 amino acid
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELVPQVTSKAFQHDTWNSGLTVASISTGHLTSDNRGDIGLLLGMIVFAVMLSILS
LIGIFNRSFRTGIKRRATNFSLLKQAGDVEENPGPMGRGLLRGLWPLHIVLWTRIASTIPP
HVQKSVNNDMIVTDNNGAVKFPQLCKFCDVRFSTCDNQKSCMSNCSITSICEKPQEVCV
AVWRKNDENITLETVCHDPKLPYHDFILEDAASPKCIMKEKKKPGETFFMCSCSSDECN
DNIIFSEEYNTSNPDLLLVIFQVSDWLIFFASLGSFLSILLVGVLGYLGLNRAAR
SEQ ID NO: 28. TGB23-1 nucleic acid
ATGGAAGCCGCCGTGGCCGCTCCCAGACCTCGACTGCTGCTGCTGGTGCTGGCCGCT
GCCGCCGCTGCCGCCGCTGCTCTGCTGCCTGGCGCCACAGCCCTGCAGTGCTTCTGT
CACCTGTGCACCAAGGACAACTTTACATGCGTGACCGATGGCCTGTGCTTCGTGTCC
GTGACAGAGACAACAGACAAGGTGATCCACAATTCTATGTGCATCGCCGAGATCGA
CCTGATCCCAAGGGATCGCCCCTTCGTGTGCGCACCAAGCTCCAAGACCGGAAGCG
TGACCACAACCTACTGCTGTAACCAGGATCACTGCAATAAGATCGAGCTGCCAACA
ACCGTGAAGTCTAGCCCTGGCCTGGGACCAGTGGAGCTGGCAGCCGTGATCGCCGG
CCCCGTGTGCTTCGTGTGCATCAGCCTGATGCTGATGGTGTACATCTGTCACAACAG
GACAGTGAATCGGTCCTTCAGAACCGGCATCAAGCGGAGAATCCTGCTGCTGATCC
CAAAGTGGCTGTATGAGGACATCCCCAACATGAAGAACTCTAATGTGGTGAAGATG
CTGCAGGAGAATAGCGAGCTGATGAACAATAACTCCTCTGAGCAGGTGCTGTATGT
GGACCCAATGATCACAGAGATCAAGGAGATCTTTATCCCCGAGCACAAGCCTACCG
ATTACAAGAAGGAGAACACAGGCCCCCTGGAGACACGGGACTATCCCCAGAACAGC
CTGTTTGATAATACAACCGTGGTGTACATCCCCGATCTGAACACCGGCTATAAGCCT
CAGATCTCCAATTTCCTGCCAGAGGGCAGCCACCTGTCCAATAACAATGAGATCACA
TCTCTGACCCTGAAGCCCCCTGTGGACTCTCTGGATAGCGGCAACAATCCCAGACTG
CAGAAGCACCCTAATTTCGCCTTTTCCGTGAGCTCCGTGAACTCTCTGAGCAATACC
ATCTTCCTGGGCGAGCTGTCTCTGATCCTGAACCAGGGCGAGTGCTCTAGCCCTGAC
ATCCAGAATAGCGTGGAGGAGGAGACAACCATGCTGCTGGAGAACGACTCCCCTTC
TGAGACAATCCCAGAGCAGACCCTGCTGCCAGATGAGTTCGTGAGCTGTCTGGGCAT
CGTGAATGAGGAGCTGCCCAGCATCAACACATACTTTCCTCAGAATATCCTGGAGTC
TCACTTCAACAGGATCAGCCTGCTGGAGAAGGCCACCAATTTCAGCCTGCTGAAGC
AGGCAGGAGATGTGGAGGAGAACCCAGGACCAATGGGAAGGGGCCTGCTGAGGGG
CCTGTGGCCTCTGCACATCGTGCTGTGGACACGCATCGCCAGCACCATCCCACCCCA
CGTGCAGAAGTCCGTGAACAATGACATGATCGTGACCGATAACAATGGCGCCGTGA
AGTTTCCCCAGCTGTGCAAGTTCTGTGACGTGCGGTTTTCCACATGCGATAACCAGA
AGTCCTGCATGTCTAATTGTAGCATCACCTCCATCTGCGAGAAGCCTCAGGAGGTGT
GCGTGGCCGTGTGGAGAAAGAACGACGAGAATATCACCCTGGAGACAGTGTGCCAC
GATCCCAAGCTGCCTTACCACGACTTCATCCTGGAGGATGCCGCCAGCCCTAAGTGT
ATCATGAAGGAGAAGAAGAAGCCAGGCGAGACATTCTTCATGTGCTCCTGTTCCTCT
GACGAGTGCAACGATAATATCATCTTTTCCGAGGAGTATAACACATCTAATCCTGAC
CTGCTGCTGGTGATCTTCCAGGTGACCGGAATCTCCCTGCTGCCTCCACTGGGAGTG
GCCATCTCTGTGATCATCATCTTCTACTGTTATAGGGTGAACCGCCAGAATAGGGCA
GCAAGACACCTGTGCCCACCTCTGCCTACACCATGTGCAAGCTCCGCCATCGAGTTT
CCAGGAGGAAAGGAGACATGGCAGTGGATCAACCCTGTGGACTTCCAGGAGGAGGC
CAGCCTGCAGGAGGCCCTGGTGGTGGAGATGTCCTGGGATAAGGGCGAGAGAACAG
AGCCACTGGAGAAGACCGAGCTGCCAGAGGGCGCCCCCGAACTGGCACTGGATACT
GAACTGAGCCTGGAGGACGGGGATAGATGTAAAGCAAAAATG
SEQ ID NO: 29. TGB23-2 nucleic acid
ATGGGAAGAGGACTGCTGAGAGGACTGTGGCCCCTGCATATCGTGCTGTGGACTCG
CATCGCTAGTACAATCCCCCCCCATGTGCAGAAGTCCGTGAACAATGACATGATCGT
GACCGATAACAATGGCGCCGTGAAGTTTCCCCAGCTGTGCAAGTTCTGTGACGTGCG
GTTTTCTACATGCGATAACCAGAAGAGCTGCATGTCCAATTGTTCTATCACCAGCAT
CTGCGAGAAGCCTCAGGAGGTGTGCGTGGCCGTGTGGAGAAAGAACGACGAGAATA
TCACCCTGGAGACAGTGTGCCACGATCCCAAGCTGCCTTACCACGACTTCATCCTGG
AGGATGCCGCCTCTCCTAAGTGTATCATGAAGGAGAAGAAGAAGCCAGGCGAGACA
TTCTTCATGTGCAGCTGTAGCTCCGACGAGTGCAACGATAACATCATCTTTTCTGAG
GAGTACAACACAAGCAATCCCGACCTGCTGCTGGTGATCTTCCAGGTGACCGGAAT
CAGCCTGCTGCCACCTCTGGGAGTGGCCATCTCCGTGATCATCATCTTTTACTGTTAT
AGGGTGAACCGCCAGAATCGGAGCTTCAGAACCGGCATCAAGCGGAGAATCCTGCT
GCTGATCCCAAAGTGGCTGTACGAGGATATCCCCAACATGAAGAACTCTAATGTGGT
GAAGATGCTGCAGGAGAATAGCGAGCTGATGAACAATAACTCTAGCGAGCAGGTGC
TGTATGTGGACCCAATGATCACAGAGATCAAGGAGATCTTTATCCCAGAGCACAAG
CCCACCGATTACAAGAAGGAGAACACAGGCCCCCTGGAGACACGGGACTATCCCCA
GAACAGCCTGTTTGATAATACCACAGTGGTGTACATCCCCGACCTGAACACAGGCTA
TAAGCCTCAGATCAGCAATTTCCTGCCAGAGGGCTCCCACCTGTCTAATAACAATGA
GATCACATCCCTGACCCTGAAGCCACCCGTGGACAGCCTGGATTCCGGCAACAATCC
CAGGCTGCAGAAGCACCCTAATTTCGCCTTTTCCGTGTCCTCTGTGAACTCTCTGAGC
AATACCATCTTCCTGGGCGAGCTGTCCCTGATCCTGAACCAGGGCGAGTGCAGCTCC
CCTGACATCCAGAACAGCGTGGAGGAGGAGACAACAATGCTGCTGGAGAACGACTC
CCCTTCTGAGACAATCCCAGAGCAGACCCTGCTGCCAGATGAGTTTGTGAGCTGTCT
GGGCATCGTGAATGAGGAGCTGCCCTCCATCAACACATACTTCCCTCAGAATATCCT
GGAGTCCCACTTTAACCGGATCTCTCTGCTGGAGAAGGCCACCAATTTCTCCCTGCT
GAAGCAGGCAGGAGATGTGGAGGAGAACCCAGGACCTATGGAGGCCGCCGTGGCC
GCCCCAAGGCCCCGCCTGCTGCTGCTGGTGCTGGCAGCCGCCGCAGCAGCAGCAGC
CGCCCTGCTGCCTGGCGCCACAGCCCTGCAGTGCTTCTGTCACCTGTGCACCAAGGA
CAACTTTACATGCGTGACCGATGGCCTGTGCTTCGTGTCTGTGACAGAGACAACAGA
CAAGGTGATCCACAATAGCATGTGCATCGCCGAGATCGACCTGATCCCTCGGGATA
GACCCTTCGTGTGCGCCCCATCTAGCAAGACCGGCAGCGTGACCACAACCTACTGCT
GTAACCAGGATCACTGCAATAAGATCGAGCTGCCAACAACCGTGAAGTCCTCTCCA
GGCCTGGGACCTGTGGAGCTGGCAGCAGTGATCGCCGGCCCCGTGTGCTTCGTGTGC
ATCTCCCTGATGCTGATGGTGTATATCTGTCACAACAGGACAGTGAATAGGGCAGCA
AGGCACCTGTGCCCTCCACTGCCTACCCCATGTGCAAGCTCCGCCATCGAGTTTCCA
GGAGGAAAGGAGACATGGCAGTGGATCAACCCTGTGGACTTCCAGGAGGAGGCCTC
CCTGCAGGAGGCCCTGGTGGTGGAGATGTCTTGGGATAAGGGCGAGCGCACAGAGC
CTCTGGAGAAGACCGAGCTGCCAGAGGGCGCCCCCGAACTGGCACTGGATACTGAA
CTGTCCCTGGAAGACGGAGATAGATGTAAAGCAAAAATG
SEQ ID NO: 30. TGB23-3 nucleic acid
ATGGAAGCTGCTGTCGCCGCTCCCCGACCTAGACTGCTGCTGCTGGTGCTGGCCGCC
GCCGCTGCTGCCGCCGCCGCTCTGCTGCCCGGCGCCACAGCCCTGCAGTGCTTCTGT
CACCTGTGCACCAAGGACAACTTTACATGCGTGACCGATGGCCTGTGCTTCGTGTCT
GTGACAGAGACAACAGACAAGGTGATCCACAATAGCATGTGCATCGCCGAGATCGA
CCTGATCCCAAGGGATCGCCCCTTCGTGTGCGCACCAAGCTCCAAGACCGGAAGCG
TGACCACAACCTACTGCTGTAACCAGGATCACTGCAATAAGATCGAGCTGCCAACA
ACCGTGAAGTCTAGCCCTGGCCTGGGACCAGTGGAGCTGGCAGCCGTGATCGCCGG
CCCCGTGTGCTTCGTGTGCATCTCCCTGATGCTGATGGTGTATATCTGTCACAACAGG
ACAGTGAATAGGGCAGCAAGACACCTGTGCCCACCTCTGCCAACCCCATGTGCATC
CTCTGCCATCGAGTTTCCTGGCGGCAAGGAGACATGGCAGTGGATCAACCCAGTGG
ACTTCCAGGAGGAGGCCAGCCTGCAGGAGGCCCTGGTGGTGGAGATGTCCTGGGAT
AAGGGCGAGCGGACAGAGCCACTGGAGAAGACCGAGCTGCCAGAGGGAGCACCTG
AGCTGGCCCTGGACACAGAGCTGTCCCTGGAGGACGGCGATAGATGCAAGGCCAAG
ATGGCCACCAACTTTTCTCTGCTGAAGCAGGCCGGCGATGTGGAGGAGAATCCTGGC
CCAATGGGAAGGGGCCTGCTGAGGGGCCTGTGGCCCCTGCACATCGTGCTGTGGAC
AAGAATCGCCTCCACCATCCCACCCCACGTGCAGAAGTCTGTGAACAATGACATGA
TCGTGACCGATAACAATGGCGCCGTGAAGTTCCCTCAGCTGTGCAAGTTCTGTGACG
TGAGGTTTAGCACATGCGATAACCAGAAGAGCTGCATGTCCAATTGTTCTATCACCA
GCATCTGCGAGAAGCCACAGGAGGTGTGCGTGGCCGTGTGGCGCAAGAACGACGAG
AATATCACCCTGGAGACAGTGTGCCACGATCCCAAGCTGCCTTACCACGACTTTATC
CTGGAGGATGCCGCCTCCCCAAAGTGTATCATGAAGGAGAAGAAGAAGCCCGGCGA
GACATTCTTCATGTGCTCTTGTAGCTCCGACGAGTGCAACGATAACATCATCTTTTCT
GAGGAGTACAACACAAGCAATCCTGACCTGCTGCTGGTGATCTTCCAGGTGACCGG
CATCTCTCTGCTGCCTCCACTGGGCGTGGCCATCAGCGTGATCATCATCTTTTACTGT
TATAGGGTGAACCGCCAGAATCGGTCCTTCAGAACCGGCATCAAGCGGAGAATCCT
GCTGCTGATCCCCAAGTGGCTGTACGAGGATATCCCTAACATGAAGAACTCCAATGT
GGTGAAGATGCTGCAGGAGAACAGCGAGCTGATGAACAATAACTCTAGCGAGCAGG
TGCTGTATGTGGACCCCATGATCACAGAGATCAAGGAGATCTTCATCCCAGAGCACA
AGCCCACCGATTACAAGAAGGAGAACACAGGCCCCCTGGAGACACGGGACTATCCC
CAGAACAGCCTGTTTGATAATACAACCGTGGTGTACATCCCTGACCTGAACACAGGC
TATAAGCCTCAGATCTCTAATTTCCTGCCAGAGGGCTCCCACCTGTCTAATAACAAT
GAGATCACAAGCCTGACCCTGAAGCCCCCTGTGGACAGCCTGGATTCCGGCAACAA
TCCCAGACTGCAGAAGCACCCTAACTTCGCCTTTTCCGTGTCCTCTGTGAACTCTCTG
AGCAATACCATCTTTCTGGGCGAGCTGAGCCTGATCCTGAACCAGGGCGAGTGCAG
CTCCCCCGACATCCAGAACAGCGTGGAGGAGGAGACAACCATGCTGCTGGAGAATG
ACTCCCCTTCTGAGACAATCCCAGAGCAGACCCTGCTGCCCGATGAGTTCGTGAGCT
GTCTGGGCATCGTGAACGAGGAGCTGCCTTCCATCAATACATACTTCCCACAGAACA
TCCTGGAGTCCCACTTCAACAGAATCTCACTGCTGGAAAAG
SEQ ID NO: 31. TGB23-4 nucleic acid
ATGGAAGCTGCCGTCGCTGCTCCCCGCCCTCGCCTGCTGCTGCTGGTCCTGGCTGCC
GCCGCTGCTGCCGCTGCTGCCCTGCTGCCCGGCGCCACAGCCCTGCAGTGCTTCTGT
CACCTGTGCACCAAGGACAACTTTACATGCGTGACCGATGGCCTGTGCTTCGTGTCT
GTGACAGAGACAACAGACAAGGTGATCCACAATAGCATGTGCATCGCCGAGATCGA
CCTGATCCCAAGGGATCGCCCCTTCGTGTGCGCACCAAGCTCCAAGACCGGATCCGT
GACCACAACCTACTGCTGTAACCAGGACCACTGTAATAAGATCGAGCTGCCCACAA
CCGTGAAGTCTAGCCCAGGCCTGGGACCAGTGGAGCTGGTGCCTCAGGTGACCAGC
AAGGCCTTTCAGCACGATACATGGAACTCCGGCCTGACCGTGGCCTCTATCAGCACA
GGCCACCTGACCTCCGACAATCGGGGCGATATCGGCCTGCTGCTGGGCATGATCGTG
TTCGCCGTGATGCTGTCTATCCTGAGCCTGATCGGCATCTTCAACCGGAGCTTTAGA
ACCGGCATCAAGCGGAGAATCCTGCTGCTGATCCCCAAGTGGCTGTACGAGGACAT
CCCTAACATGAAGAACTCTAATGTGGTGAAGATGCTGCAGGAGAATAGCGAGCTGA
TGAACAATAACTCCTCTGAGCAGGTGCTGTATGTGGACCCCATGATCACAGAGATCA
AGGAGATCTTTATCCCTGAGCACAAGCCAACCGATTACAAGAAGGAGAACACAGGC
CCCCTGGAGACACGGGACTATCCTCAGAACTCCCTGTTTGATAATACAACCGTGGTG
TACATCCCTGATCTGAACACCGGCTATAAGCCACAGATCAGCAATTTCCTGCCCGAG
GGCTCCCACCTGTCTAATAACAATGAGATCACATCCCTGACCCTGAAGCCCCCTGTG
GACAGCCTGGATTCCGGCAACAATCCTCGCCTGCAGAAGCACCCAAATTTCGCCTTT
AGCGTGAGCTCCGTGAACTCCCTGTCTAATACCATCTTCCTGGGCGAGCTGTCCCTG
ATCCTGAACCAGGGCGAGTGCTCTAGCCCAGACATCCAGAACAGCGTGGAGGAGGA
GACAACCATGCTGCTGGAGAACGACAGCCCATCCGAGACAATCCCCGAGCAGACCC
TGCTGCCCGATGAGTTTGTGAGCTGTCTGGGCATCGTGAATGAGGAGCTGCCTTCCA
TCAACACATACTTTCCACAGAATATCCTGGAGAGCCACTTCAACCGGATCTCCCTGC
TGGAGAAGGCCACCAATTTCTCCCTGCTGAAGCAGGCAGGAGATGTGGAGGAGAAC
CCAGGACCTATGGGAAGGGGCCTGCTGAGAGGCCTGTGGCCCCTGCACATCGTGCT
GTGGACAAGAATCGCCTCCACCATCCCACCCCACGTGCAGAAGTCTGTGAACAATG
ACATGATCGTGACCGATAACAATGGCGCCGTGAAGTTCCCTCAGCTGTGCAAGTTCT
GTGACGTGAGGTTTAGCACATGCGATAACCAGAAGTCTTGCATGAGCAATTGTTCCA
TCACCTCTATCTGCGAGAAGCCACAGGAGGTGTGCGTGGCCGTGTGGCGCAAGAAC
GACGAGAATATCACCCTGGAGACAGTGTGCCACGATCCTAAGCTGCCATATCACGA
CTTTATCCTGGAGGATGCCGCCTCTCCAAAGTGTATCATGAAGGAGAAGAAGAAGC
CCGGCGAGACATTCTTTATGTGCAGCTGTTCCTCTGACGAGTGCAACGATAATATCA
TCTTCTCTGAGGAGTACAACACCAGCAATCCAGACCTGCTGCTGGTGATCTTTCAGG
TGAGCGATTGGCTGATCTTCTTTGCCAGCCTGGGCTCCTTCCTGTCTATCCTGCTGGT
GGGCGTGCTGGGATATCTGGGCCTGAACAGGGCAGCAAGGCACCTGTGCCCTCCAC
TGCCAACACCCTGTGCCAGCTCCGCCATCGAGTTTCCTGGCGGCAAGGAGACATGGC
AGTGGATCAATCCAGTGGACTTCCAGGAGGAGGCCTCCCTGCAGGAGGCCCTGGTG
GTGGAGATGTCTTGGGATAAGGGCGAGAGGACAGAGCCTCTGGAGAAGACCGAGCT
GCCCGAGGGCGCCCCTGAACTGGCACTGGATACTGAACTGAGCCTGGAGGATGGAG
ATAGATGTAAAGCAAAGATG
SEQ ID NO: 32. TGB23-5 nucleic acid
ATGGGCAGGGGGCTGCTGCGGGGGCTGTGGCCTCTGCATATTGTGCTGTGGACTCGG
ATTGCTTCAACTATTCCTCCTCATGTCCAGAAGTCTGTGAACAATGACATGATCGTG
ACCGATAACAATGGCGCCGTGAAGTTTCCCCAGCTGTGCAAGTTCTGTGACGTGAGG
TTTAGCACATGCGATAACCAGAAGTCTTGCATGAGCAATTGTTCCATCACCTCTATC
TGCGAGAAGCCTCAGGAGGTGTGCGTGGCCGTGTGGCGCAAGAACGACGAGAATAT
CACCCTGGAGACAGTGTGCCACGATCCTAAGCTGCCATACCACGACTTTATCCTGGA
GGATGCCGCCAGCCCAAAGTGTATCATGAAGGAGAAGAAGAAGCCCGGCGAGACA
TTCTTCATGTGCTCCTGTAGCTCCGACGAGTGCAACGATAATATCATCTTCAGCGAG
GAGTATAACACATCCAATCCTGACCTGCTGCTGGTGATCTTTCAGGTGCCACAGGTG
ACCTCCAAGGCCTTCCAGCACGATACATGGAACTCTGGCCTGACCGTGGCCTCTATC
AGCACAGGCCACCTGACCTCCGACAATCGGGGCGATATCGGCCTGCTGCTGGGCAT
GATCGTGTTTGCCGTGATGCTGAGCATCCTGTCCCTGATCGGCATCTTCAACAGGAG
CTTTCGCACCGGCATCAAGCGGAGAATCCTGCTGCTGATCCCCAAGTGGCTGTACGA
GGACATCCCTAACATGAAGAACAGCAATGTGGTGAAGATGCTGCAGGAGAACAGCG
AGCTGATGAACAATAACTCTAGCGAGCAGGTGCTGTATGTGGACCCAATGATCACA
GAGATCAAGGAGATCTTCATCCCCGAGCACAAGCCTACCGATTACAAGAAGGAGAA
CACAGGCCCCCTGGAGACACGGGACTATCCTCAGAACTCCCTGTTTGATAATACCAC
AGTGGTGTACATCCCTGATCTGAACACAGGCTATAAGCCACAGATCTCCAATTTCCT
GCCCGAGGGCTCTCACCTGAGCAATAACAATGAGATCACATCTCTGACCCTGAAGC
CCCCTGTGGACTCCCTGGATTCTGGCAACAATCCTAGACTGCAGAAGCACCCAAATT
TCGCCTTTAGCGTGTCCTCTGTGAACTCCCTGTCTAATACCATCTTCCTGGGCGAGCT
GTCTCTGATCCTGAACCAGGGCGAGTGCAGCTCCCCTGACATCCAGAATAGCGTGG
AGGAGGAGACAACAATGCTGCTGGAGAACGACAGCCCATCCGAGACAATCCCCGA
GCAGACCCTGCTGCCAGATGAGTTCGTGAGCTGTCTGGGCATCGTGAATGAGGAGCT
GCCTTCTATCAACACATACTTCCCACAGAATATCCTGGAGTCTCACTTTAACAGGAT
CAGCCTGCTGGAGAAGGCCACCAATTTCAGCCTGCTGAAGCAGGCAGGAGACGTGG
AGGAGAACCCTGGACCAATGGAGGCCGCCGTGGCCGCCCCCAGGCCTCGCCTGCTG
CTGCTGGTGCTGGCAGCCGCCGCAGCAGCAGCAGCCGCCCTGCTGCCAGGAGCAAC
AGCCCTGCAGTGCTTCTGTCACCTGTGCACCAAGGACAACTTTACATGCGTGACCGA
TGGCCTGTGCTTCGTGAGCGTGACAGAGACAACAGATAAGGTGATCCACAATTCCAT
GTGCATCGCCGAGATCGACCTGATCCCACGGGATAGACCCTTCGTGTGCGCCCCATC
TAGCAAGACCGGCTCCGTGACCACAACCTACTGCTGTAACCAGGACCACTGTAATA
AGATCGAGCTGCCCACAACCGTGAAGTCCTCTCCTGGCCTGGGACCAGTGGAGCTG
GTGAGCGATTGGCTGATCTTCTTTGCCAGCCTGGGCTCCTTCCTGTCTATCCTGCTGG
TGGGCGTGCTGGGATATCTGGGCCTGAACAGGGCAGCAAGACACCTGTGCCCACCA
CTGCCAACACCATGTGCAAGCTCCGCCATCGAGTTTCCAGGAGGAAAGGAGACATG
GCAGTGGATCAATCCTGTGGACTTCCAGGAGGAGGCCTCTCTGCAGGAGGCCCTGGT
GGTGGAGATGAGCTGGGATAAGGGCGAGCGCACAGAGCCCCTGGAGAAGACCGAG
CTGCCCGAGGGCGCCCCTGAACTGGCACTGGATACTGAACTGAGCCTGGAAGATGG
CGATAGGTGTAAAGCAAAAATG
SEQ ID NO: 33. TGB23-6 nucleic acid
ATGGAAGCCGCTGTGGCCGCTCCCCGACCTCGACTGCTGCTGCTGGTGCTGGCTGCC
GCTGCTGCTGCCGCTGCCGCTCTGCTGCCTGGCGCCACAGCCCTGCAGTGCTTCTGT
CACCTGTGCACCAAGGACAACTTTACATGCGTGACCGATGGCCTGTGCTTCGTGAGC
GTGACAGAGACAACAGACAAGGTGATCCACAATTCCATGTGCATCGCCGAGATCGA
CCTGATCCCAAGGGATCGCCCCTTCGTGTGCGCACCAAGCTCCAAGACCGGCTCCGT
GACCACAACCTACTGCTGTAACCAGGACCACTGTAATAAGATCGAGCTGCCCACAA
CCGTGAAGTCTAGCCCAGGCCTGGGACCAGTGGAGCTGGTGAGCGATTGGCTGATC
TTCTTTGCCTCTCTGGGCAGCTTCCTGTCCATCCTGCTGGTGGGCGTGCTGGGATATC
TGGGCCTGAACAGGGCAGCAAGACACCTGTGCCCACCTCTGCCCACACCTTGTGCCT
CCTCTGCCATCGAGTTTCCTGGCGGCAAGGAGACATGGCAGTGGATCAATCCAGTGG
ACTTCCAGGAGGAGGCCTCCCTGCAGGAGGCCCTGGTGGTGGAGATGTCTTGGGAT
AAGGGCGAGAGGACAGAGCCACTGGAGAAGACCGAGCTGCCTGAGGGAGCACCAG
AGCTGGCCCTGGACACAGAGCTGTCTCTGGAGGACGGCGATCGCTGCAAGGCCAAG
ATGGCCACCAACTTTAGCCTGCTGAAGCAGGCCGGCGATGTGGAGGAGAATCCTGG
ACCAATGGGAAGGGGCCTGCTGAGAGGCCTGTGGCCTCTGCACATCGTGCTGTGGA
CACGGATCGCCTCTACCATCCCACCCCACGTGCAGAAGAGCGTGAACAATGACATG
ATCGTGACCGATAACAATGGCGCCGTGAAGTTCCCTCAGCTGTGCAAGTTCTGTGAC
GTGCGGTTTAGCACATGCGATAACCAGAAGTCTTGCATGAGCAATTGTTCCATCACC
TCTATCTGCGAGAAGCCACAGGAGGTGTGCGTGGCCGTGTGGAGAAAGAACGACGA
GAATATCACCCTGGAGACAGTGTGCCACGATCCTAAGCTGCCATACCACGACTTTAT
CCTGGAGGATGCCGCCTCTCCCAAGTGTATCATGAAGGAGAAGAAGAAGCCTGGCG
AGACATTCTTCATGTGCAGCTGTAGCTCCGACGAGTGTAACGATAATATCATCTTCA
GCGAGGAGTATAACACATCCAATCCAGACCTGCTGCTGGTGATCTTTCAGGTGCCCC
AGGTGACCAGCAAGGCCTTCCAGCACGATACATGGAACTCCGGCCTGACCGTGGCC
TCTATCAGCACAGGCCACCTGACCTCCGACAATAGGGGCGATATCGGCCTGCTGCTG
GGCATGATCGTGTTTGCCGTGATGCTGAGCATCCTGTCCCTGATCGGCATCTTCAAC
AGGAGCTTTCGCACCGGCATCAAGCGGAGAATCCTGCTGCTGATCCCTAAGTGGCTG
TACGAGGACATCCCAAACATGAAGAACTCTAATGTGGTGAAGATGCTGCAGGAGAA
TAGCGAGCTGATGAACAATAACTCTAGCGAGCAGGTGCTGTATGTGGACCCCATGA
TCACAGAGATCAAGGAGATCTTCATCCCCGAGCACAAGCCTACCGATTACAAGAAG
GAGAACACAGGCCCCCTGGAGACACGGGACTATCCTCAGAACTCCCTGTTTGATAA
TACAACCGTGGTGTACATCCCTGATCTGAACACCGGCTATAAGCCACAGATCAGCA
ATTTCCTGCCCGAGGGCTCTCACCTGAGCAATAACAATGAGATCACATCCCTGACCC
TGAAGCCTCCAGTGGACTCCCTGGATTCTGGCAACAATCCTCGCCTGCAGAAGCACC
CAAACTTCGCCTTTAGCGTGTCCTCTGTGAACTCCCTGTCTAATACCATCTTTCTGGG
CGAGCTGTCCCTGATCCTGAACCAGGGCGAGTGCAGCTCCCCCGACATCCAGAACA
GCGTGGAGGAGGAGACAACCATGCTGCTGGAGAATGACAGCCCATCCGAGACAATC
CCCGAGCAGACCCTGCTGCCCGATGAGTTCGTGTCCTGTCTGGGCATCGTGAACGAG
GAGCTGCCTTCTATCAATACATACTTCCCTCAGAACATCCTGGAGAGCCACTTCAAT
AGAATTAGCCTGCTGGAGAAA
SEQ ID NO: 34. TGB23-14 nucleic acid
ATGGAAGCTGCCGTGGCTGCTCCAAGACCTAGACTGCTGCTGCTGGTGCTGGCCGCC
GCCGCCGCCGCTGCCGCTGCCCTGCTGCCCGGCGCCACAGCCCTGCAGTGCTTCTGT
CACCTGTGCACCAAGGACAACTTTACATGCGTGACCGATGGCCTGTGCTTCGTGTCT
GTGACAGAGACAACAGACAAGGTGATCCACAATAGCATGTGCATCGCCGAGATCGA
CCTGATCCCAAGGGATCGCCCCTTCGTGTGCGCACCAAGCTCCAAGACCGGCTCCGT
GACCACAACCTACTGCTGTAACCAGGACCACTGTAATAAGATCGAGCTGCCCACAA
CCGTGAAGTCTAGCCCAGGCCTGGGACCAGTGGAGCTGGTGCCTCAGGTGACCAGC
AAGGCCTTTCAGCACGATACATGGAACTCCGGCCTGACCGTGGCCTCTATCAGCACA
GGCCACCTGACCTCCGACAATCGGGGCGATATCGGCCTGCTGCTGGGCATGATCGTG
TTCGCCGTGATGCTGTCTATCCTGAGCCTGATCGGCATCTTCAACCGGAGCTTTAGA
ACCGGCATCAAGCGGAGAATCCTGCTGCTGATCCCCAAGTGGCTGTTTGAGGACATC
CCTAACATGAAGAACTCTAATGTGGTGAAGATGCTGCAGGAGAATAGCGAGCTGAT
GAACAATAACTCCTCTGAGCAGGTGCTGTTCGTGGACCCCATGATCACAGAGATCAA
GGAGATCTTTATCCCTGAGCACAAGCCAACCGATTTCAAGAAGGAGAACACAGGCC
CCCTGGAGACACGGGACTTCCCTCAGAACTCCCTGTTTGATAATACAACCGTGGTGT
TCATCCCTGATCTGAACACCGGCTTCAAGCCACAGATCAGCAATTTTCTGCCCGAGG
GCTCCCACCTGTCTAATAACAATGAGATCACATCCCTGACCCTGAAGCCCCCTGTGG
ACAGCCTGGATTCCGGCAACAATCCTCGCCTGCAGAAGCACCCAAATTTCGCCTTTA
GCGTGAGCTCCGTGAACTCCCTGTCTAATACCATCTTTCTGGGCGAGCTGTCCCTGAT
CCTGAACCAGGGCGAGTGCTCTAGCCCAGACATCCAGAACAGCGTGGAGGAGGAGA
CAACCATGCTGCTGGAGAACGACAGCCCATCCGAGACAATCCCCGAGCAGACCCTG
CTGCCCGATGAGTTCGTGAGCTGTCTGGGCATCGTGAATGAGGAGCTGCCTTCCATC
AACACATTCTTTCCACAGAATATCCTGGAGAGCCACTTTAACCGGATCTCCCTGCTG
GAGAAGGCCACCAATTTCTCCCTGCTGAAGCAGGCAGGAGATGTGGAGGAGAACCC
AGGACCTATGGGAAGGGGCCTGCTGAGAGGCCTGTGGCCCCTGCACATCGTGCTGT
GGACAAGAATCGCCTCCACCATCCCACCCCACGTGCAGAAGTCTGTGAACAATGAC
ATGATCGTGACCGATAACAATGGCGCCGTGAAGTTCCCTCAGCTGTGCAAGTTCTGT
GACGTGAGGTTTAGCACATGCGATAACCAGAAGTCTTGCATGAGCAATTGTTCCATC
ACCTCTATCTGCGAGAAGCCACAGGAGGTGTGCGTGGCCGTGTGGCGCAAGAACGA
CGAGAATATCACCCTGGAGACAGTGTGCCACGATCCTAAGCTGCCATATCACGACTT
TATCCTGGAGGATGCCGCCTCTCCAAAGTGTATCATGAAGGAGAAGAAGAAGCCCG
GCGAGACATTCTTTATGTGCAGCTGTTCCTCTGACGAGTGCAACGATAATATCATCT
TCTCTGAGGAGTACAACACCAGCAATCCAGACCTGCTGCTGGTGATCTTTCAGGTGA
GCGATTGGCTGATCTTCTTTGCCAGCCTGGGCTCCTTCCTGTCTATCCTGCTGGTGGG
CGTGCTGGGATATCTGGGCCTGAACAGGGCAGCAAGGCACCTGTGCCCTCCACTGC
CAACACCCTGTGCCAGCTCCGCCATCGAGTTTCCTGGCGGCAAGGAGACATGGCAG
TGGATCAATCCAGTGGACTTCCAGGAGGAGGCCTCCCTGCAGGAGGCCCTGGTGGT
GGAGATGTCTTGGGATAAGGGCGAGAGGACAGAGCCTCTGGAGAAGACCGAGCTGC
CCGAGGGCGCCCCTGAGCTGGCACTGGACACCGAACTGAGTCTGGAGGATGGCGAC
CGCTGTAAGGCTAAAATG
SEQ ID NO: 35. TGB23-15 nucleic acid
ATGGAAGCCGCCGTCGCCGCTCCCCGCCCAAGACTGCTGCTGCTGGTCCTGGCCGCT
GCCGCTGCTGCCGCCGCCGCACTGCTGCCCGGCGCCACAGCCCTGCAGTGCTTCTGT
CACCTGTGCACCAAGGACAACTTCACCTGCGTGACCGATGGCCTGTGCTTCGTGTCC
GTGACAGAGACAACAGACAAGGTGATCCACAACTCTATGTGCATCGCCGAGATCGA
CCTGATCCCAAGGGATCGCCCCTTCGTGTGCGCACCTAGCTCCAAGACCGGCTCCGT
GACCACAACCTACTGCTGTAACCAGGACCACTGTAATAAGATCGAGCTGCCCACAA
CCGTGAAGTCTAGCCCAGGCCTGGGACCAGTGGAGCTGGTGCCTCAGGTGACCTCT
AAGGCCTTCCAGCACGATACATGGAATAGCGGCCTGACCGTGGCCAGCATCTCCAC
AGGCCACCTGACCTCTGACAACAGGGGCGATATCGGCCTGCTGCTGGGCATGATCG
TGTTTGCCGTGATGCTGTCTATCCTGAGCCTGATCGGCATCTTCAACCGGAGCTTTAG
AACAGGCATCAAGCGGAGAGCCACCAATTTCTCCCTGCTGAAGCAGGCAGGAGATG
TGGAGGAGAACCCTGGACCAATGGGAAGGGGCCTGCTGAGGGGCCTGTGGCCACTG
CACATCGTGCTGTGGACACGGATCGCCAGCACCATCCCCCCTCACGTGCAGAAGTCC
GTGAACAATGACATGATCGTGACCGATAACAATGGCGCCGTGAAGTTTCCACAGCT
GTGCAAGTTCTGTGACGTGCGGTTTAGCACATGCGATAACCAGAAGTCCTGCATGTC
TAATTGTAGCATCACCTCCATCTGCGAGAAGCCACAGGAGGTGTGCGTGGCCGTGTG
GAGAAAGAACGACGAGAATATCACCCTGGAGACAGTGTGCCACGATCCTAAGCTGC
CATACCACGACTTTATCCTGGAGGATGCCGCCTCTCCTAAGTGTATCATGAAGGAGA
AGAAGAAGCCAGGCGAGACATTCTTTATGTGCAGCTGTTCCTCTGACGAGTGCAACG
ATAATATCATCTTCTCCGAGGAGTATAACACCTCTAATCCCGACCTGCTGCTGGTGA
TCTTTCAGGTGTCCGATTGGCTGATCTTCTTTGCCTCTCTGGGCAGCTTCCTGTCCAT
CCTGCTGGTGGGGGTCCTGGGCTACCTGGGGCTGAATCGGGCTGCTCGG
SEQ ID NO: 36. huLIC19309b CAR amino acid
MALPVTALLLPLALLLHAARPDIVMTQSPLSLPVTPGEPASISCRSSQTIVHSNGNTYLEW
YLQKPGQSPQLLIYKVSNRFSGVPDRFSGSGSGTDFTLKISRVEAEDVGVYYCFQVSHVP
YTFGGGTKLEIKGSTSGSGKPGSGEGSTKGQVQLVQSGAEVKKPGASVKVSCKASGYTF
TDYEMHWVRQATGQGLEWMGGIDPETGNTAYSQKFKGRVTMTRNTSISTAYMELSSL
RSEDTAVYYCARYYSFAYWGQGTLVTVSSTSTTTPAPRPPTPAPTIASQPLSLRPEACRP
AAGGAVHTRGLDFACDIYIWAPLAGTCGVLLLSLVITLYCKRGRKKLLYIFKQPFMRPV
QTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQ
GLSTATKDTYDALHMQALPPR
SEQ ID NO: 37. huLIC19309bT CAR amino acid
MALPVTALLLPLALLLHAARPDIVMTQSPLSLPVTPGEPASISCRSSQTIVHSNGNTYLEW
YLQKPGQSPQLLIYKVSNRFSGVPDRFSGSGSGTDFTLKISRVEAEDVGVYYCFQVSHVP
YTFGGGTKLEIKGSTSGSGKPGSGEGSTKGQVQLVQSGAEVKKPGASVKVSCKASGYTF
TDYEMHWVRQATGQGLEWMGGIDPETGNTAYSQKFKGRVTMTRNTSISTAYMELSSL
RSEDTAVYYCARYYSFAYWGQGTLVTVSSTSTTTPAPRPPTPAPTIASQPLSLRPEACRP
AAGGAVHTRGLDFACDIYIWAPLAGTCGVLLLSLVITLYCKRGRKKLLYIFKQPFMRPV
QTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQ
GLSTATKDTYDALHMQALPPRVNGSGATNFSLLKQAGDVEENPGPMEAAVAAPRPRLL
LLVLAAAAAAAAALLPGATALQCFCHLCTKDNFTCVTDGLCFVSVTETTDKVIHNSMCI
AEIDLIPRDRPFVCAPSSKTGSVTTTYCCNQDHCNKIELPTTVKSSPGLGPVELVSDWLIF
FASLGSFLSILLVGVLGYLGLNRAARHLCPPLPTPCASSAIEFPGGKETWQWINPVDFQEE
ASLQEALVVEMSWDKGERTEPLEKTELPEGAPELALDTELSLEDGDRCKAKMATNFSLL
KQAGDVEENPGPMGRGLLRGLWPLHIVLWTRIASTIPPHVQKSVNNDMIVTDNNGAVK
FPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVP
QVTSKAFQHDTWNSGLTVASISTGHLTSDNRGDIGLLLGMIVFAVMLSILSLIGIFNRSFR
TGIKRRILLLIPKWLYEDIPNMKNSNVVKMLQENSELMNNNSSEQVLYVDPMITEIKEIFI
PEHKPTDYKKENTGPLETRDYPQNSLFDNTTVVYIPDLNTGYKPQISNFLPEGSHLSNNN
EITSLTLKPPVDSLDSGNNPRLQKHPNFAFSVSSVNSLSNTIFLGELSLILNQGECSSPDIQ
NSVEEETTMLLENDSPSETIPEQTLLPDEFVSCLGIVNEELPSINTYFPQNILESHENRISLL
EK
SEQ ID NO: 38. huLIC19309bT CAR nucleic acid
atggccctgcctgtgaccgctctgctgctgcctctggctctgctgctgcacgctgcccgccctgacattgtgatgacccagtctcccctgtcc
ctgccagtgacccctggagagccagcctccatctcttgcaggagctcccagaccatcgtgcacagcaacggcaatacatacctggagtgg
tatctgcagaagcccggccagtcccctcagctgctgatctacaaggtgtctaacaggttcagcggagtgccagaccgctttagoggatccg
gatctggcaccgatttcacactgaagatctctagagtggaggccgaggacgtgggcgtgtactattgcttccaggtgagccacgtgccatat
acctttggcggcggcacaaagctggagatcaagggcagcacctccggatctggcaagccaggatccggagagggatctacaaagggac
aggtgcagctggtgcagagcggagcagaggtgaagaagcctggagccagcgtgaaggtgtcctgtaaggcctctggctacaccttcaca
gactatgagatgcactgggtgcggcaggcaaccggacagggcctggagtggatgggaggaatcgacccagagacaggcaacacagcc
tactcccagaagtttaagggccgggtgaccatgacaagaaataccagcatctccacagcctatatggagctgtctagcctgagatctgagga
tacagccgtgtactattgtgctcgctactattcctttgcctactggggacaggggacactggtcaccgtctcatcaactagtaccacgacgc
cagcgccgcgaccaccaacaccggcgcccaccatcgcgtcgcagcccctgtccctgcgcccagaggcgtgccggccagcggcgggggg
cgcagtgcacacgagggggctggacttcgcctgtgatatctacatctgggcgcccttggccgggacttgtggggtccttctcctgtcactgg
ttatcaccctttactgcaaacggggcagaaagaaactcctgtatatattcaaacaaccatttatgagaccagtacaaactactcaagaggaa
gatggctgtagctgccgatttccagaagaagaagaaggaggatgtgaactgagagtgaagttcagcaggagcgcagacgcccccgcgtac
cagcagggccagaaccagctctataacgagctcaatctaggacgaagagaggagtacgatgttttggacaagagacgtggccgggaccc
tgagatggggggaaagccgagaaggaagaaccctcaggaaggcctgtacaatgaactgcagaaagataagatggcggaggcctacagt
gagattgggatgaaaggcgagcgccggaggggcaaggggcacgatggcctttaccagggtctcagtacagccaccaaggacacctac
gacgcccttcacatgcaggccctgccccctcgcgttaacggcagcggagctacaaacttcagtcttctaaaacaggctggtgacgtggagg
agaatcccggccctatggaggccgccgtggcggctcctcggccccgcctcctcctgctggtgctggccgcagctgctgccgccgcggct
gccttgctgcctggcgccaccgcgctgcagtgcttttgtcacctatgcactaaggacaacttcacctgcgtgaccgacggcctgtgcttcgt
gtccgtaacagagactaccgataaagtgattcacaactccatgtgcatcgccgagattgacctgatcccgcgcgaccgccccttcgtctgtg
ctccctcatccaagaccggcagcgtcaccacaacttactgctgcaaccaggaccactgtaacaagatcgagctgcccacaacagtcaaatcc
tctcctggattgggacccgtggagctggtgagtgactggttgatcttctttgcgagcctaggctccttcttaagcatccttcttgtgggcgt
cctgggttacctgggactgaaccgcgctgcgcgccacctgtgcccgcctctgccaaccccctgcgcctcctcggccatcgagttccccgggg
ggaaggagacctggcaatggatcaatccagtagactttcaggaggaggcatcgctgcaggaggctctggtggtggagatgagttgggacaa
gggtgaacggaccgaaccactcgagaaaaccgagctgccagaaggtgcgccggagctggccctggatactgagctttcactggaggac
ggcgacaggtgtaaggccaaaatggccaccaactttagcctgctgaaacaggccggtgatgtggaggagaatccgggcccaatgggcc
gcggcctcctacgtggactgtggcccttgcacatcgtgctttggacccgcatcgcttctaccatcccacctcatgtgcagaagagcgttaata
acgacatgatcgtcactgataacaacggggcggtgaagttccctcagctgtgcaagttctgtgatgtacgcttctctacttgcgacaaccaga
agagttgtatgtctaactgctctattacttctatctgcgagaagccccaggaggtctgcgtggccgtttggaggaagaatgacgagaacatca
ctctggagacagtatgccatgatccgaagctgccctatcatgacttcatcctggaggatgccgcaagccctaagtgcatcatgaaggagaa
gaagaagccgggggagaccttcttcatgtgctcctgctcttccgatgagtgcaacgacaacatcatcttttccgaggagtacaacacttccaa
ccccgatctgctgttggtgattttccaggtgccgcaggtcacctccaaggcgttccagcatgacacctggaacagcggactaaccgtggcta
gcatcagcaccggccacttgacctccgacaaccgcggggacatcggcctgctactcggaatgatcgtgttcgcggtgatgctctccatcct
ctctctcatcggcatcttcaaccgcagcttccgcaccggtattaagaggagaattctgcttctcatccccaaatggttatacgaggacatccc
caacatgaaaaacagcaacgtggtcaagatgctgcaggagaattcggagctgatgaacaataactcatcggagcaggttctgtacgtggacc
cgatgatcacggaaataaaggagatcttcatccctgagcacaagcctaccgactacaagaaagagaataccggcccgcttgagactcgtg
actatcctcagaactctctgttcgacaacacgacggtggtgtacattcccgacttgaacacgggctacaaaccccaaatttccaacttcctgc
ctgagggcagtcacctgtccaacaacaacgaaatcacctccctgaccctgaagccaccggtcgactccttggacageggcaacaaccccc
gactccagaagcaccccaattttgctttttccgtgtcctccgtcaactccctctcaaataccatcttccteggegagctctccctgatcctga
atcagggcgaatgttcgtccccggacatccagaactctgtggaggaggagaccacgatgcttctggagaacgacagcccgtccgagaccatc
cccgagcagacgcttctgcccgacgagttcgtgagttgcctgggcattgtgaacgaggaactgccttctattaacacctacttccctcagaa
catactggagagccactttaaccgtatctcgctattggagaag
SEQ ID NO: 39. CD8α signal peptide
MALPVTALLLPLALLLHAARP
SEQ ID NO: 40. CD8α hinge
TTTPAPRPPTPAPTIASQPLSLRPEACRPAAGGAVHTRGLDFACD
SEQ ID NO: 41. CD8α transmembrane domain
IYIWAPLAGTCGVLLLSLVITLYC
SEQ ID NO: 42. 4-1BB (CD137) co-stimulatory signaling domain
KRGRKKLLYIFKQPFMRPVQTTQEEDGCSCRFPEEEEGGCEL
SEQ ID NO: 43. CD3ζ (CD3z) intracellular signaling domain
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 44. EcoRI restriction site
GAATTC
SEQ ID NO: 45. SpeI restriction site
ACTAGT
SEQ ID NO: 46. HpaI restriction site
GTTAAC
SEQ ID NO: 47. MluI restriction site
ACGCGT
SEQ ID NO: 48. P2A
ATNFSLLKQAGDVEENPGP
SEQ ID NO: 49. TGFβR1EC-TGFβR1TM-IL23RIC
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELAAVIAGPVCFVCISLMLMVYICHNRTVNRSFRTGIKRRILLLIPKWLYEDIPNM
KNSNVVKMLQENSELMNNNSSEQVLYVDPMITEIKEIFIPEHKPTDYKKENTGPLETRDY
PQNSLFDNTTVVYIPDLNTGYKPQISNFLPEGSHLSNNNEITSLTLKPPVDSLDSGNNPRL
QKHPNFAFSVSSVNSLSNTIFLGELSLILNQGECSSPDIQNSVEEETTMLLENDSPSETIPEQ
TLLPDEFVSCLGIVNEELPSINTYFPQNILESHENRISLLEK
SEQ ID NO: 50. TGFβR2EC-TGFβR2TM-IL12Rβ1IC
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVTGISLLPPLGVAIS
VIIIFYCYRVNRQNRAARHLCPPLPTPCASSAIEFPGGKETWQWINPVDFQEEASLQEALV
VEMSWDKGERTEPLEKTELPEGAPELALDTELSLEDGDRCKAKM
SEQ ID NO: 51. TGFβR1EC-TGFβR1TM-IL12Rβ1IC
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELAAVIAGPVCFVCISLMLMVYICHNRTVNRAARHLCPPLPTPCASSAIEFPGGK
ETWQWINPVDFQEEASLQEALVVEMSWDKGERTEPLEKTELPEGAPELALDTELSLEDG
DRCKAKM
SEQ ID NO: 52. TGFβR2EC-TGFβR2TM-IL23RIC
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVTGISLLPPLGVAIS
VIIIFYCYRVNRQNRSFRTGIKRRILLLIPKWLYEDIPNMKNSNVVKMLQENSELMNNNS
SEQVLYVDPMITEIKEIFIPEHKPTDYKKENTGPLETRDYPQNSLFDNTTVVYIPDLNTGY
KPQISNFLPEGSHLSNNNEITSLTLKPPVDSLDSGNNPRLQKHPNFAFSVSSVNSLSNTIFL
GELSLILNQGECSSPDIQNSVEEETTMLLENDSPSETIPEQTLLPDEFVSCLGIVNEELPSIN
TYFPQNILESHENRISLLEK
SEQ ID NO: 53. TGFβR1EC-IL23RTM-IL23RIC
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELVPQVTSKAFQHDTWNSGLTVASISTGHLTSDNRGDIGLLLGMIVFAVMLSILS
LIGIFNRSFRTGIKRRILLLIPKWLYEDIPNMKNSNVVKMLQENSELMNNNSSEQVLYVD
PMITEIKEIFIPEHKPTDYKKENTGPLETRDYPQNSLFDNTTVVYIPDLNTGYKPQISNFLP
EGSHLSNNNEITSLTLKPPVDSLDSGNNPRLQKHPNFAFSVSSVNSLSNTIFLGELSLILNQ
GECSSPDIQNSVEEETTMLLENDSPSETIPEQTLLPDEFVSCLGIVNEELPSINTYFPQNILE
SHENRISLLEK
SEQ ID NO: 54. TGFβR2EC-IL12Rβ1TM-IL12Rβ1IC
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVSDWLIFFASLGSFL
SILLVGVLGYLGLNRAARHLCPPLPTPCASSAIEFPGGKETWQWINPVDFQEEASLQEAL
VVEMSWDKGERTEPLEKTELPEGAPELALDTELSLEDGDRCKAKM
SEQ ID NO: 55.
TGFβR2EC-IL23RTM-IL23RIC
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVPQVTSKAFQHDT
WNSGLTVASISTGHLTSDNRGDIGLLLGMIVFAVMLSILSLIGIFNRSFRTGIKRRILLLIPK
WLYEDIPNMKNSNVVKMLQENSELMNNNSSEQVLYVDPMITEIKEIFIPEHKPTDYKKE
NTGPLETRDYPQNSLFDNTTVVYIPDLNTGYKPQISNFLPEGSHLSNNNEITSLTLKPPVD
SLDSGNNPRLQKHPNFAFSVSSVNSLSNTIFLGELSLILNQGECSSPDIQNSVEEETTMLLE
NDSPSETIPEQTLLPDEFVSCLGIVNEELPSINTYFPQNILESHENRISLLEK
SEQ ID NO: 56.
TGFβR1EC-IL12Rβ1TM-IL12Rβ1IC
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELVSDWLIFFASLGSFLSILLVGVLGYLGLNRAARHLCPPLPTPCASSAIEFPGGK
ETWQWINPVDFQEEASLQEALVVEMSWDKGERTEPLEKTELPEGAPELALDTELSLEDG
DRCKAKM
SEQ ID NO: 57. linker
SGSGKPGSGEGSTKG
SEQ ID NO: 58. Anti-GPC3 scFv
DIVMTQSPLSLPVTPGEPASISCRSSQTIVHSNGNTYLEWYLQKPGQSPQLLIYKVSNRFS
GVPDRFSGSGSGTDFTLKISRVEAEDVGVYYCFQVSHVPYTFGGGTKLEIKGSTSGSGKP
GSGEGSTKGQVQLVQSGAEVKKPGASVKVSCKASGYTFTDYEMHWVRQATGQGLEW
MGGIDPETGNTAYSQKFKGRVTMTRNTSISTAYMELSSLRSEDTAVYYCARYYSFAYW
GQGTLVTVSS
SEQ ID NO: 59. Kozak sequence
GCCGCCACC
SEQ ID NO: 60. P2A
GSGATNFSLLKQAGDVEENPGP
SEQ ID NO: 61. Linker
(G)ₙ
(where n is an integer of at least one)
SEQ ID NO: 62 Linker
(GS)ₙ
(where n is an integer of at least one)
SEQ ID NO: 63 Linker
(GSGGS)ₙ
(where n is an integer of at least one)
SEQ ID NO: 64 Linker
(GGGGS)ₙ
(where n is an integer of at least one)
SEQ ID NO: 65 Linker
(GGGS)ₙ
(where n is an integer of at least one)
SEQ ID NO: 66 TGFβR1 isoform-1
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTVKSSP
GLGPVELAAVIAGPVCFVCISLMLMVYICHNRTVIHHRVPNEEDPSLDRPFISEGTTLKDL
IYDMTTSGSGSGLPLLVQRTIARTIVLQESIGKGRFGEVWRGKWRGEEVAVKIFSSREER
SWFREAEIYQTVMLRHENILGFIAADNKDNGTWTQLWLVSDYHEHGSLFDYLNRYTVT
VEGMIKLALSTASGLAHLHMEIVGTQGKPAIAHRDLKSKNILVKKNGTCCIADLGLAVR
HDSATDTIDIAPNHRVGTKRYMAPEVLDDSINMKHFESFKRADIYAMGLVFWEIARRCS
IGGIHEDYQLPYYDLVPSDPSVEEMRKVVCEQKLRPNIPNRWQSCEALRVMAKIMREC
WYANGAARLTALRIKKTLSQLSQQEGIKM
SEQ ID NO: 67 TGFβR1 isoform-2
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTGPFSV
KSSPGLGPVELAAVIAGPVCFVCISLMLMVYICHNRTVIHHRVPNEEDPSLDRPFISEGTT
LKDLIYDMTTSGSGSGLPLLVQRTIARTIVLQESIGKGRFGEVWRGKWRGEEVAVKIFSS
REERSWFREAEIYQTVMLRHENILGFIAADNKDNGTWTQLWLVSDYHEHGSLFDYLNR
YTVTVEGMIKLALSTASGLAHLHMEIVGTQGKPAIAHRDLKSKNILVKKNGTCCIADLG
LAVRHDSATDTIDIAPNHRVGTKRYMAPEVLDDSINMKHFESFKRADIYAMGLVFWEIA
RRCSIGGIHEDYQLPYYDLVPSDPSVEEMRKVVCEQKLRPNIPNRWQSCEALRVMAKIM
RECWYANGAARLTALRIKKTLSQLSQQEGIKM
SEQ ID NO: 68 TGFβR1 isoform-3
TTDKVIHNSMCIAEIDLIPRDRPFVCAPSSKTGSVTTTYCCNQDHCNKIELPTTVKSSPGL
GPVELAAVIAGPVCFVCISLMLMVYICHNRTVIH
SEQ ID NO: 69 TGFβR1 isoform-4
MEAAVAAPRPRLLLLVLAAAAAAAAALLPGATALQCFCHLCTKDNFTCVTDGLCFVSV
TETTDKVIHNSMCIAEIDLIPRDRPFVCAPSSKTGSVTTTYCCNQDHCNKIELPTTGLPLL
VQRTIARTIVLQESIGKGRFGEVWRGKWRGEEVAVKIFSSREERSWFREAEIYQTVMLR
HENILGFIAADNKDNGTWTQLWLVSDYHEHGSLFDYLNRYTVTVEGMIKLALSTASGL
AHLHMEIVGTQGKPAIAHRDLKSKNILVKKNGTCCIADLGLAVRHDSATDTIDIAPNHR
VGTKRYMAPEVLDDSINMKHFESFKRADIYAMGLVFWEIARRCSIGGIHEDYQLPYYDL
VPSDPSVEEMRKVVCEQKLRPNIPNRWQSCEALRVMAKIMRECWYANGAARLTALRIK
KTLSQLSQQEGIKM
SEQ ID NO: 70 TGFβR2 isoform-1
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLLVIFQVTGISLLPPLGVAIS
VIIIFYCYRVNRQQKLSSTWETGKTRKLMEFSEHCAIILEDDRSDISSTCANNINHNTELLP
IELDTLVGKGRFAEVYKAKLKQNTSEQFETVAVKIFPYEEYASWKTEKDIFSDINLKHEN
ILQFLTAEERKTELGKQYWLITAFHAKGNLQEYLTRHVISWEDLRKLGSSLARGIAHLHS
DHTPCGRPKMPIVHRDLKSSNILVKNDLTCCLCDFGLSLRLDPTLSVDDLANSGQVGTA
RYMAPEVLESRMNLENVESFKQTDVYSMALVLWEMTSRCNAVGEVKDYEPPFGSKVR
EHPCVESMKDNVLRDRGRPEIPSFWLNHQGIQMVCETLTECWDHDPEARLTAQCVAER
FSELEHLDRLSGRSCSEEKIPEDGSLNTTK
SEQ ID NO: 71 TGFβR2 isoform-2
MGRGLLRGLWPLHIVLWTRIASTIPPHVPKSDVEMEAQKDASIHLSCNRTIHPLKHENSD
VMASDNGGAVKLPQLCKFCDVRLSTCDNQKSCMSNCSITAICEKPHEVCVAVWRKND
KNITLETVCHDPKLTYHGFTLEDAASPKCVMKEKKRAGETFFMCACNMEECNDYIIFSE
EYTTSSPDLLLVIIQVTGVSLLPPLGIAIAVIIIFYCYRVHRQQKLSPSWESSKPRKLMDFS
DNCAIILEDDRSDISSTCANNINHNTELLPIELDTLVGKGRFAEVYKAKLKQNTSEQFETV
AVKIFPYEEYSSWKTEKDIFSDINLKHENILQFLTAEERKTELGKQYWLITAFHAKGNLQ
EYLTRHVISWEDLRKLGSSLARGIAHLHSDHTPCGRPKMPIVHRDLKSSNILVKNDLTCC
LCDFGLSLRLDPTLSVDDLANSGQVGTARYMAPEVLESRMNLENVESFKQTDVYSMAL
VLWEMTSRCNAVGEVKDYEPPFGSKVREHPCVESMKDSVLRDRGRPEIPSFWLNHQGI
QIVCETLTECWDHDPEARLTAQCVAERFSELEHPERLSGRSCSQEKIPEDGSLNTTK
SEQ ID NO: 72 TGFβR2 isoform-3
MGRGLLRGLWPLHIVLWTRIASTIPPHVQKSDVEMEAQKDEIICPSCNRTAHPLRHINND
MIVTDNNGAVKFPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYN
TSNPDLLLVIFQVTGISLLPPLGVAISVIIIFYCYRVNRQQKLSSTWETGKTRKLMEFSEHC
AIILEDDRSDISSTCANNINHNTELLPIELDTLVGKGRFAEVYKAKLKQNTSEQFETVAVK
IFPYEEYASWKTEKDIFSDINLKHENILQFLTAEERKTELGKQYWLITAFHAKGNLQEYL
TRHVISWEDLRKLGSSLARGIAHLHSDHTPCGRPKMPIVHRDLKSSNILVKNDLTCCLCD
FGLSLRLDPTLSVDDLANSGQVGTARYMAPEVLESRMNLENVESFKQTDVYSMALVL
WEMTSRCNAVGEVKDYEPPFGSKVREHPCVESMKDNVLRDRGRPEIPSFWLNHQGIQM
VCETLTECWDHDPEARLTAQCVAERFSELEHLDRLSGRSCSEEKIPEDGSLNTTK
SEQ ID NO: 73 IL-23R isoform-1
MNQVTIQWDAVIALYILFSWCHGGITNINCSGHIWVEPATIFKMGMNISIYCQAAIKNCQ
PRKLHFYKNGIKERFQITRINKTTARLWYKNFLEPHASMYCTAECPKHFQETLICGKDIS
SGYPPDIPDEVTCVIYEYSGNMTCTWNAGKLTYIDTKYVVHVKSLETEEEQQYLTSSYIN
ISTDSLQGGKKYLVWVQAANALGMEESKQLQIHLDDIVIPSAAVISRAETINATVPKTIIY
WDSQTTIEKVSCEMRYKATTNQTWNVKEFDTNFTYVQQSEFYLEPNIKYVFQVRCQET
GKRYWQPWSSPFFHKTPETVPQVTSKAFQHDTWNSGLTVASISTGHLTSDNRGDIGLLL
GMIVFAVMLSILSLIGIFNRSFRTGIKRRILLLIPKWLYEDIPNMKNSNVVKMLQENSELM
NNNSSEQVLYVDPMITEIKEIFIPEHKPTDYKKENTGPLETRDYPQNSLFDNTTVVYIPDL
NTGYKPQISNFLPEGSHLSNNNEITSLTLKPPVDSLDSGNNPRLQKHPNFAFSVSSVNSLS
NTIFLGELSLILNQGECSSPDIQNSVEEETTMLLENDSPSETIPEQTLLPDEFVSCLGIVNEE
LPSINTYFPQNILESHENRISLLEK
SEQ ID NO: 74 IL-23R isoform-2
MRYKATTNQTWNVKEFDTNFTYVQQSEFYLEPNIKYVFQVRCQETGKRYWQPWSSPFF
HKTPETVPQVTSKAFQHDTWNSGLTVASISTGHLTSDNRGDIGLLLGMIVFAVMLSILSLI
GIFNRSFRTGIKRRILLLIPKWLYEDIPNMKNSNVVKMLQENSELMNNNSSEQVLYVDP
MITEIKEIFIPEHKPTDYKKENTGPLETRDYPQNSLFDNTTVAYIPDLDTGYKPQISNFLPE
GSHLSNNNEITSLTLKPPVDSLDSGNNPRLQKHPNFAFSVSSVNSLSNTIFLGELSLILNQG
ECSSPDIQNSVEEETTMLLENDSPSETIPEQTLLPDEFVSCLGIVNEELPSINTYFPQNILES
HENRISLLEK
SEQ ID NO: 75 IL-23R isoform-3
MEFWANSCFHLYRAPYFWIKRRILLLIPKWLYEDIPNMKNSNVVKMLQENSELMNNNS
SEQVLYVDPMITEIKEIFIPEHKPTDYKKENTGPLETRDYPQNSLFDNTTVVYIPDLNTGY
KPQISNFLPEGSHLSNNNEITSLTLKPPVDSLDSGNNPRLQKHPNFAFSVSSVNSLSNTIFL
GELSLILNQGECSSPDIQNSVEEETTMLLENDSPRETIPEQTLLPDEFVSCLGIVNEELPSIN
TYFPQNILESHENRISLLEK
SEQ ID NO: 76 IL-23R isoform-4
MILRPYQPCGTVKEFDTNFTYVQQSEFYLEPNIKYVFQVRCQETGKRYWQPWSSLFFHK
TPETVPQVTSKAFQHDTWNSGLTVASISTGHLTSDNRGDIGLLLGMIVFAVMLSILSLIGI
FNRSFRTGIKRRILLLIPKWLYEDIPNMKNSNVVKMLQENSELMNNNSSEQVLYVDPMIT
EIKEIFIPEHKPTDYKKENTGPLETRDYPQNSLFDNTTVVYIPDLNTGYKPQISNFLPEGSH
LSNNNEITSLTLKPPVDSLDSGNNPRLQKHPNFAFSVSSVNSLSNTIFLGELSLILNQGECS
SPDIQNSVEEETTMLLENDSPSETIPEQTLLPDEFVSCLGIVNEELPSINTYFPQNILESHEN
RISLLEK
SEQ ID NO: 77 IL-23R isoform-5
MKNSNVVKMLQENSELMNNNSSEQVLYVDPMITEIKEIFIPEHKPTDYKKENTGPLETR
DYPQNSLFDNTTVVYIPDLNTGYKPQISNFLPEGSHLSNNNEITSLTLKPPVDSLDSGNNP
RLQKHPNFAFSVSSVNSLSNTIFLGELSLILNQGECSSPDIQNSVEEETTMLLENDSPSETIP
EQTLLPDEFVSCLGIVNEELPSINTYFPQNILESHENRISLLEK
SEQ ID NO: 78 IL-12Rβ1 isoform-1
MEPLVTWVVPLLFLFLLSRQGAACRTSECCFQDPPYPDADSGSASGPRDLRCYRISSDRY
ECSWQYEGPTAGVSHFLRCCLSSGRCCYFAAGSATRLQFSDQAGVSVLYTVTLWVESW
ARNQTEKSPEVTLQLYNSVKYEPPLGDIKVSKLAGQLRMEWETPDNQVGAEVQFRHRT
PSSPWKLGDCGPQDDDTESCLCPLEMNVAQEFQLRRRQLGSQGSSWSKWSSPVCVPPE
NPPQPQVRFSVEQLGQDGRRRLTLKEQPTQLELPEGCQGLAPGTEVTYRLQLHMLSCPC
KAKATRTLHLGKMPYLSGAAYNVAVISSNQFGPGLNQTWHIPADTHTEPVALNISVGTN
GTTMYWPARAQSMTYCIEWQPVGQDGGLATCSLTAPQDPDPAGMATYSWSRESGAM
GQEKCYYITIFASAHPEKLTLWSTVLSTYHFGGNASAAGTPHHVSVKNHSLDSVSVDW
APSLLSTCPGVLKEYVVRCRDEDSKQVSEHPVQPTETQVTLSGLRAGVAYTVQVRADT
AWLRGVWSQPQRFSIEVQVSDWLIFFASLGSFLSILLVGVLGYLGLNRAARHLCPPLPTP
CASSAIEFPGGKETWQWINPVDFQEEASLQEALVVEMSWDKGERTEPLEKTELPEGAPE
LALDTELSLEDGDRCKAKM
SEQ ID NO: 79 IL-12Rβ1 isoform-2
MEPLVTWVVPLLFLFLLSRQGAACRTSECCFQDPPYPDADSGSASGPRDLRCYRISSDRY
ECSWQYEGPTAGVSHFLRCCLSSGRCCYFAAGSATRLQFSDQAGVSVLYTVTLWVESW
ARNQTEKSPEVTLQLYNSVKYEPPLGDIKVSKLAGQLRMEWETPDNQVGAEVQFRHRT
PSSPWKLGDCGPQDDDTESCLCPLEMNVAQEFQLRRRQLGSQGSSWSKWSSPVCVPPE
NPPQPQVRFSVEQLGQDGRRRLTLKEQPTQLELPEGCQGLAPGTEVTYRLQLHMLSCPC
KAKATRTLHLGKMPYLSGAAYNVAVISSNQFGPGLNQTWHIPADTHTEPVALNISVGTN
GTTMYWPARAQSMTYCIEWQPVGQDGGLATCSLTAPQDPDPAGMATYSWSRESGAM
GQEKCYYITIFASAHPEKLTLWSTVLSTYHFGGNASAAGTPHHVSVKNHSLDSVSVDW
APSLLSTCPGVLKEYVVRCRDEDSKQVSEHPVQPTETQVTLSGLRAGVAYTVQVRADT
AWLRGVWSQPQRFSIEVQVSDWLIFFASLGSFLSILLVGVLGYLGLNRAARHLCPPLPTP
CASSAIEFPGGKETWQWINPVDFQEEASLQEALVVEMSWDKGERTEPLEKTELPEGAPE
LALDTELSLEDGDRCDR
SEQ ID NO: 80 Anti-GPC3 VH (CDR sequences per Kabat definition are bolded)
QVQLVQSGAEVKKPGASVKVSCKASGYTFTDYEMHWVRQATGQGLEWMGGIDPETG
NTAYSQKFKGRVTMTRNTSISTAYMELSSLRSEDTAVYYCARYYSFAYWGQGTLVTV
SS
SEQ ID NO: 81 Anti-GPC3 VL (CDR sequences per Kabat definition are bolded)
DIVMTQSPLSLPVTPGEPASISCRSSQTIVHSNGNTYLEWYLQKPGQSPQLLIYKVSNRF
SGVPDRFSGSGSGTDFTLKISRVEAEDVGVYYCFQVSHVPYTFGGGTKLEIK
SEQ ID NO: 82 Anti-GPC3 VH-CDR1
DYEMH
SEQ ID NO: 83 Anti-GPC3 VH-CDR2
GIDPETGNTAYSQKFKG
SEQ ID NO: 84 Anti-GPC3 VH-CDR3
YYSFAY
SEQ ID NO: 85 Anti-GPC3 VL-CDR1
RSSQTIVHSNGNTYLE
SEQ ID NO: 86 Anti-GPC3 VL-CDR2
KVSNRFS
SEQ ID NO: 87 Anti-GPC3 VL-CDR3
FQVSHVPYT
SEQ ID NO: 88 TGFβR2 isoform-4
MGRGLLRGLWPLHIVLWTRIASTIPPHVPKSVNSDVMASDNGGAVKLPQLCKFCDVRLS
TCDNQKSCMSNCSITAICEKPHEVCVAVWRKNDKNITLETVCHDPKLTYHGFTLEDAAS
PKCVMKEKKRAGETFFMCACNMEECNDYIIFSEEYTTSSPDLLLVIIQVTGVSLLPPLGIA
IAVIIIFYCYRVHRQQKLSPSWESSKPRKLMDFSDNCAIILEDDRSDISSTCANNINHNTEL
LPIELDTLVGKGRFAEVYKAKLKQNTSEQFETVAVKIFPYEEYSSWKTEKDIFSDINLKH
ENILQFLTAEERKTELGKQYWLITAFHAKGNLQEYLTRHVISWEDLRKLGSSLARGIAHL
HSDHTPCGRPKMPIVHRDLKSSNILVKNDLTCCLCDFGLSLRLDPTLSVDDLANSGQVG
TARYMAPEVLESRMNLENVESFKQTDVYSMALVLWEMTSRCNAVGEVKDYEPPFGSK
VREHPCVESMKDSVLRDRGRPEIPSFWLNHQGIQIVCETLTECWDHDPEARLTAQCVAE
RFSELEHPERLSGRSCSQEKIPEDGSLNTTK
SEQ ID NO: 89 CD20 CAR amino acid
MALPVTALLLPLALLLHAARPEVQLQQSGAELVKPGASVKMSCKASGYTFTSYNMHW
VKQTPGQGLEWIGAIYPGNGDTSYNQKFKGKATLTADKSSSTAYMQLSSLTSEDSADY
YCARSNYYGSSYWFFDVWGAGTTVTVSSGGGGSGGGGSGGGGSDIVLTQSPAILSASP
GEKVTMTCRASSSVNYMDWYQKKPGSSPKPWIYATSNLASGVPARFSGSGSGTSYSLTI
SRVEAEDAATYYCQQWSFNPPTFGGGTKLEIKTSTTTPAPRPPTPAPTIASQPLSLRPEAC
RPAAGGAVHTRGLDFACDIYIWAPLAGTCGVLLLSLVITLYCKRGRKKLLYIFKQPFMR
PVQTTQEEDGCSCRFPEEEEGGCELGENLYFQSGGDTQALLRNDQVYQPLRDRDDAQY
SHLGGNGGSGERPPPVPNPDYEPIRKGQRDLYSGLNQRGGSGDKQTLLPNDQLYQPLKD
REDDQYSHLQGNGGSGRKQRITETESPYQELQGQRSDVYSDLNTQGGSG
SEQ ID NO: 90 LCAR-UL186S nucleic acid
ATGGGCTCCAGCAACTCCAAGAGGCAGCAACAGGGCTTGCTCAAGCTCTGGCGAGG
GCTGCGAGGGAAGCCTGGGGCAGACTGGGTGCTATTGTCCGATCCGCTTATCGGGC
AGTCATCAACAGTCCAAGAAGAGTGCGGCAAGGCCTTGAAAAAGTCCTGGGGTAAA
GGTAAAATGACTCCAGACGGCCGCCGCCTGCAAGAAGGAGACACCTTTGATGAGTG
GGATGATGATGAAGAAGAAGTAGGCTTCCCTGTGCAACCTCGAGTCCCCTTAAGAC
AGATGACCTATAAATTAGCAGTGGACTTTTCCCACTTTTTAAAATCAAAGGGGGGAC
TGGATGGGATATATTACTCTGAAAGAAGAGAAAAGATCCTGAATTTGTATGCCTTGA
ACGAGTGGGGAATAATAGATGATTGGCAAGCTTACTCACCAGGCCCGGGGATAAGG
TACCCGAGAGTCTTTGGCTTCTGCTTTAAGCTAGTCCCAGTGGACCTGCATGAGGAG
GCACGCAACTGTGAGAGACACTGTGCTGCACATCCAGCACAGATGGGGGAAGATCC
TGATGGAATAGATCATGGAGAAGTCTTGGTCTGGAAGTTTGACCCGAAGTTGGCGGT
GGAGTACCGCCCGGACATGTTTAAGGACATGCACGAACATGCAAAGCGCTGAACGC
GTGCCCCTCTCCCTCCCCCCCCCCTAACGTTACTGGCCGAAGCCGCTTGGAATAAGG
CCGGTGTGCGTTTGTCTATATGTTATTTTCCACCATATTGCCGTCTTTTGGCAATGTG
AGGGCCCGGAAACCTGGCCCTGTCTTCTTGACGAGCATTCCTAGGGGTCTTTCCCCT
CTCGCCAAAGGAATGCAAGGTCTGTTGAATGTCGTGAAGGAAGCAGTTCCTCTGGA
AGCTTCTTGAAGACAAACAACGTCTGTAGCGACCCTTTGCAGGCAGCGGAACCCCC
CACCTGGCGACAGGTGCCTCTGCGGCCAAAAGCCACGTGTATAAGATACACCTGCA
AAGGCGGCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGGTACCCC
ATTGTATGGGATCTGATCTGGGGCCTCGGTGCACATGCTTTACATGTGTTTAGTCGA
GGTTAAAAAAACGTCTAGGCCCCCCGAACCACGGGGACGTGGTTTTCCTTTGAAAA
ACACGATGATAATATGGCCACAGGATCCGCCGCCACCATGGCCCTGCCAGTGACCG
CCTTGCTCCTTCCCCTGGCTCTTCTGCTGCACGCTGCTAGACCTGAGGTGCAGCTGCA
GCAGAGCGGAGCTGAGCTGGTGAAGCCTGGCGCTAGCGTGAAGATGAGCTGCAAGG
CCAGCGGCTACACCTTCACCAGCTATAACATGCACTGGGTGAAGCAGACCCCTGGA
CAGGGACTGGAGTGGATCGGAGCTATCTACCCTGGAAACGGAGACACCTCATACAA
CCAGAAGTTCAAGGGAAAGGCTACCCTGACCGCTGACAAGAGCAGCAGCACCGCTT
ACATGCAGCTGAGCTCACTGACCAGCGAGGACTCCGCCGACTACTACTGCGCCAGA
AGCAACTACTACGGAAGCAGCTACTGGTTCTTCGACGTGTGGGGAGCTGGAACCAC
CGTGACCGTGTCAAGCGGCGGCGGAGGCTCCGGAGGCGGAGGATCTGGCGGCGGCG
GCAGCGACATCGTGCTGACCCAGAGCCCTGCTATCCTGTCTGCCAGCCCTGGAGAGA
AGGTGACCATGACCTGCAGAGCTAGCAGCAGCGTGAACTACATGGACTGGTATCAG
AAAAAGCCCGGCAGCTCACCTAAGCCTTGGATCTACGCTACCAGCAACTTAGCCAG
CGGCGTGCCTGCTAGATTCTCCGGAAGCGGCTCTGGAACCAGCTACTCCCTTACCAT
CAGCAGAGTGGAGGCTGAGGACGCTGCTACCTACTACTGCCAGCAGTGGAGCTTCA
ACCCTCCTACCTTCGGAGGAGGAACCAAGCTGGAGATCAAGACTAGTACCACGACG
CCAGCGCCGCGACCACCAACACCGGCGCCCACCATCGCGTCGCAGCCCCTGTCCCTG
CGCCCAGAGGCGTGCCGGCCAGCGGCGGGGGGCGCAGTGCACACGAGGGGGCTGG
ACTTCGCCTGTGATATCTACATCTGGGCGCCCTTGGCCGGGACTTGTGGGGTCCTTCT
CCTGTCACTGGTTATCACCCTTTACTGCAAACGGGGCAGAAAGAAACTCCTGTATAT
ATTCAAACAACCATTTATGAGACCAGTACAAACTACTCAAGAGGAAGATGGCTGTA
GCTGCCGATTTCCAGAAGAAGAAGAAGGAGGATGTGAACTGGGTGAAAATTTGTAT
TTTCAATCTGGTGGTGACACACAAGCTCTGTTGAGGAATGACCAGGTCTATCAGCCC
CTCCGAGATCGAGATGATGCTCAGTACAGCCACCTTGGAGGAAACGGTGGTTCTGGT
GAGAGGCCACCACCTGTTCCCAACCCAGACTATGAGCCCATCCGGAAAGGCCAGCG
GGACCTGTATTCTGGCCTGAATCAGAGAGGTGGTTCTGGTGACAAGCAGACTCTGTT
GCCCAATGACCAGCTCTACCAGCCCCTCAAGGATCGAGAAGATGACCAGTACAGCC
ACCTTCAAGGAAACGGTGGTTCTGGTCGGAAACAGCGTATCACTGAGACCGAGTCG
CCTTATCAGGAGCTCCAGGGTCAGAGGTCGGATGTCTACAGCGACCTCAACACACA
GGGTGGTTCTGGTTAA

We claim:

1. A fusion protein comprising:
a) a first polypeptide comprising i) a first extracellular domain comprising an extracellular domain of one of TGFβR1 or TGFβR2, ii) a first transmembrane domain, and iii) a first intracellular domain comprising an intracellular domain of one of IL-12Rβ1 or IL-23R; and
b) a second polypeptide comprising i) a second extracellular domain comprising an extracellular domain of the other of TGFβR1 or TGFβR2, ii) a second transmembrane domain, and iii) a second intracellular domain comprising an intracellular domain of the other of IL-12Rβ1 or IL-23R.

2. The fusion protein of claim 1, wherein the first transmembrane domain and the second transmembrane domain each comprises a transmembrane domain of one of TGFβR1, TGFβR2, IL-12Rβ1 and IL-23R.

3. The fusion protein of claim 2, wherein the first transmembrane domain and the second transmembrane domain each comprises a transmembrane domain of one of IL-12Rβ1 or IL-23R.

4. The fusion protein of any one of claims 1-3, wherein:
a) the first extracellular domain comprises an extracellular domain of TGFβR1, the first transmembrane domain comprises a transmembrane domain of TGFβR1, the first intracellular domain comprises an intracellular domain of IL-23R, the second extracellular domain comprises an extracellular domain of TGFβR2, the second transmembrane domain comprises a transmembrane domain of TGFβR2, and the second intracellular domain comprises an intracellular domain of IL-12Rβ1;
b) the first extracellular domain comprises an extracellular domain of TGFβR2, the first transmembrane domain comprises a transmembrane domain of TGFβR2, the first intracellular domain comprises an intracellular domain of IL-23R, the second extracellular domain comprises an extracellular domain of TGFβR1, the second transmembrane domain comprises a transmembrane domain of TGFβR1, and the second intracellular domain comprises an intracellular domain of IL-12Rβ1;
c) the first extracellular domain comprises an extracellular domain of TGFβR1, the first transmembrane domain comprises a transmembrane domain of IL-23R, the first intracellular domain comprises an intracellular domain of IL-23R, the second extracellular domain comprises an extracellular domain of TGFβR2, the second transmembrane domain comprises a transmembrane domain of IL-12Rβ1, and the second intracellular domain comprises an intracellular domain of IL-12Rβ1; or
d) the first extracellular domain comprises an extracellular domain of TGFβR2, the first transmembrane domain comprises a transmembrane domain of IL-23R, the first intracellular domain comprises an intracellular domain of IL-23R, the second extracellular domain comprises an extracellular domain of TGFβR1, the second transmembrane domain comprises a transmembrane domain of IL-12Rβ1, and the second intracellular domain comprises an intracellular domain of IL-12Rβ1.

5. The fusion protein of any one of claims 1-4, wherein the first extracellular domain comprises an extracellular domain of TGFβR2, the first transmembrane domain comprises a transmembrane domain of IL-23R, the first intracellular domain comprises an intracellular domain of IL-23R, the second extracellular domain comprises an extracellular domain of TGFβR1, the second transmembrane domain comprises a transmembrane domain of IL-12Rβ1, and the second intracellular domain comprises an intracellular domain of IL-12Rβ1.

6. The fusion protein of any one of claims 1-5, wherein the first polypeptide and/or the second polypeptide further comprises a signal peptide at the N-terminus of the polypeptide.

7. The fusion protein of any one of claims 1-6, wherein the first polypeptide and the second polypeptide are in a single polypeptide, and wherein the first polypeptide and the second polypeptide are separated by a multicistronic element.

8. The fusion protein of claim 7, wherein the multicistronic element comprises a 2A self-cleaving peptide selected from the group consisting of T2A, P2A, E2A, or F2A.

9. The fusion protein of claim 7 or claim 8, wherein the first polypeptide is N-terminal to the second polypeptide.

10. The fusion protein of claim 7 or claim 8, wherein the first polypeptide is C-terminal to the second polypeptide.

11. The fusion protein of any one of claims 7-9, wherein the single polypeptide comprises, from N-terminus to C-terminus:
a) the first extracellular domain comprising an extracellular domain of TGFβR1, the first transmembrane domain comprising a transmembrane domain of TGFβR1, the first intracellular domain comprising an intracellular domain of IL-23R, a 2A self-cleaving peptide, the second extracellular domain comprising an extracellular domain of TGFβR2, the second transmembrane domain comprising a transmembrane domain of TGFβR2, and the second intracellular domain comprising an intracellular domain of IL-12Rβ1;
b) the first extracellular domain comprising an extracellular domain of TGFβR2, the first transmembrane domain comprising a transmembrane domain of TGFβR2, the first intracellular domain comprising an intracellular domain of IL-23R, a 2A self-cleaving peptide, the second extracellular domain comprising an extracellular domain of TGFβR1, the second transmembrane domain comprising a transmembrane domain of TGFβR1, and the second intracellular domain comprising an intracellular domain of IL-12Rβ1;
c) the first extracellular domain comprising an extracellular domain of TGFβR1, the first transmembrane domain comprising a transmembrane domain of TGFβR1, the first intracellular domain comprising an intracellular domain of IL-12Rβ1, a 2A self-cleaving peptide, the second extracellular domain comprising an extracellular domain of TGFβR2, the second transmembrane domain comprising a transmembrane domain of TGFβR2, and the second intracellular domain comprising an intracellular domain of IL-23R;
d) the first extracellular domain comprising an extracellular domain of TGFβR1, the first transmembrane domain comprising a transmembrane domain of IL-23R, the first intracellular domain comprising an intracellular domain of IL-23R, a 2A self-cleaving peptide, the second extracellular domain comprising an extracellular domain of TGFβR2, the second transmembrane domain comprising a transmembrane domain of IL-12Rβ1, and the second intracellular domain comprising an intracellular domain of IL-12Rβ1;
e) the first extracellular domain comprising an extracellular domain of TGFβR2, the first transmembrane domain comprising a transmembrane domain of IL-23R, the first intracellular domain comprising an intracellular domain of IL-23R, a 2A self-cleaving peptide, the second extracellular domain comprising an extracellular domain of TGFβR1, the second transmembrane domain comprising a transmembrane domain of IL-12Rβ1, and the second intracellular domain comprising an intracellular domain of IL-12Rβ1; or
f) the first extracellular domain comprising an extracellular domain of TGFβR1, the first transmembrane domain comprising a transmembrane domain of IL-12Rβ1, the first intracellular domain comprising an intracellular domain of IL-12Rβ1, a 2A self-cleaving peptide, the second extracellular domain comprising an extracellular domain of TGFβR2, the second transmembrane domain comprising a transmembrane domain of IL-23R, and the second intracellular domain comprising an intracellular domain of IL-23R.

12. The fusion protein of any one of claims 1-6, wherein the first extracellular domain and the second extracellular domain form a binding site for TGFβ, wherein the first intracellular domain and the second intracellular domain form an IL-23 receptor complex, and wherein upon binding of the fusion protein to TGFβ, signaling through the IL-23 receptor complex is transmitted.

13. The fusion protein of any one of claims 2-12, wherein:
a) the transmembrane domain of IL-23R comprises the amino acid sequence of SEQ ID NO: 7, or a functional variant having at least about 90% sequence identity;
b) the transmembrane domain of IL-12Rβ1 comprises the amino acid sequence of SEQ ID NO: 8, or a functional variant having at least about 90% sequence identity;
c) the transmembrane domain of TGFβR1 comprises the amino acid sequence of SEQ ID NO: 5, or a functional variant having at least about 90% sequence identity; and/or
d) the transmembrane domain of TGFβR2 comprises the amino acid sequence of SEQ ID NO: 6, or a functional variant having at least about 90% sequence identity.

14. The fusion protein of any one of claims 1-13, wherein:
a) the extracellular domain of TGFβR1 comprises the amino acid sequence of SEQ ID NO: 3, or a functional variant having at least about 90% sequence identity;
b) the extracellular domain of TGFβR2 comprises the amino acid sequence of SEQ ID NO: 4, or a functional variant having at least about 90% sequence identity;
c) the intracellular domain of IL-23R comprises the amino acid sequence of SEQ ID NO: 15, or a functional variant having at least about 90% sequence identity; and/or
d) the intracellular domain of IL-12Rβ1 comprises the amino acid sequence of SEQ ID NO: 16, or a functional variant having at least about 90% sequence identity.

15. The fusion protein of any one of claims 1-14, wherein:
1) the fusion protein comprises a) a first polypeptide comprising i) a first extracellular domain comprising the amino acid sequence of SEQ ID NO: 3, ii) a first transmembrane domain comprising the amino acid sequence of SEQ ID NO: 5, iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 6, and iii) a second intracellular domain comprising SEQ ID NO: 16;
2) the fusion protein comprises a) a first polypeptide comprising i) a first extracellular domain comprising the amino acid sequence of SEQ ID NO: 4, ii) a first transmembrane domain comprising the amino acid sequence of SEQ ID NO: 6, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 5, and iii) a second intracellular domain comprising the amino acid sequence of SEQ ID NO: 16;
3) the fusion protein comprises a) a first polypeptide comprising i) a first extracellular domain comprising the amino acid sequence of SEQ ID NO: 3, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 4, ii) a second transmembrane domain comprising the amino acid sequence of SEQ ID NO: 8, and iii) a second intracellular domain comprising the amino acid sequence of SEQ ID NO: 16; or
4) the fusion protein comprises a) a first polypeptide comprising i) a first extracellular domain comprising the amino acid sequence of SEQ ID NO: 4, ii) a first transmembrane domain comprising the amino acid sequence of SEQ ID NO: 7, and iii) a first intracellular domain comprising the amino acid sequence of SEQ ID NO: 15; and b) a second polypeptide comprising i) a second extracellular domain comprising the amino acid sequence of SEQ ID NO: 3, ii) a second transmembrane domain comprising the amino acid sequence of SEQ ID NO: 8, and iii) a second intracellular domain comprising the amino acid sequence of SEQ ID NO: 16.

16. The fusion protein of any one of claims 1-15, wherein the first polypeptide and/or the second polypeptide further comprises a linker between two domains, wherein one of the two domains is the transmembrane domain, and the other domain is the extracellular domain or the intracellular domain.

17. The fusion protein of claim 16, wherein the linker comprises a membrane proximal sequence, wherein the membrane proximal sequence and the transmembrane domain are derived from the same molecule.

18. The fusion protein of claim 17, wherein the membrane proximal region comprises an amino acid sequence set forth in any of SEQ ID NOs: 9-12.

19. The fusion protein of any one of claims 1-18, wherein:
a) the first polypeptide comprises the amino acid sequence of SEQ ID NO: 49, and the second polypeptide comprises the amino acid sequence of SEQ ID NO: 50;
b) the first polypeptide comprises the amino acid sequence of SEQ ID NO: 50, and the second polypeptide comprises the amino acid sequence of SEQ ID NO: 49;
c) the first polypeptide comprises the amino acid sequence of SEQ ID NO: 51, and the second polypeptide comprises the amino acid sequence of SEQ ID NO: 52;
d) the first polypeptide comprises the amino acid sequence of SEQ ID NO: 53, and the second polypeptide comprises the amino acid sequence of SEQ ID NO: 54;
e) the first polypeptide comprises the amino acid sequence of SEQ ID NO: 55, and the second polypeptide comprises the amino acid sequence of SEQ ID NO: 56; or
f) the first polypeptide comprises the amino acid sequence of SEQ ID NO: 56, and the second polypeptide comprises the amino acid sequence of SEQ ID NO: 55.

20. The fusion protein of any one of claims 1-19, wherein the fusion protein comprises an amino acid sequence set forth in any of SEQ ID NOs: 20-25.

21. A nucleic acid comprising one or more nucleic acid sequences encoding the fusion protein of any of claims 1-18 or a portion thereof.

22. A nucleic acid comprising a nucleic acid sequence set forth in any of SEQ ID NOs: 28-33.

23. The nucleic acid of claim 21 or claim 22, wherein the nucleic acid further comprises a second nucleic acid sequence encoding a functional exogenous receptor, wherein the functional exogenous receptor comprises an extracellular ligand-binding domain and optionally an intracellular signaling domain.

24. The nucleic acid of claim 23, wherein the functional exogenous receptor is selected from the group consisting of: an engineered T cell receptor (TCR), a chimeric antigen receptor (CAR), a T cell antigen coupler (TAC) or a portion thereof.

25. The nucleic acid of claim 23 or claim 24, wherein the functional exogenous receptor specifically recognizes a tumor antigen.

26. The nucleic acid of any of claims 23-35, wherein the functional exogenous receptor comprises a chimeric antigen receptor (CAR) that specifically recognizes a tumor antigen.

27. The nucleic acid of claim 26, wherein the CAR specifically recognizes CD19, CD20, CD22, CD30, CD33, CD38, BCMA, CS1, CD138, CD123/IL3Rα, c-Met, gp100, MUC1, IGF-I receptor, EpCAM, EGFR/EGFRvIII, HER2, IGF1R, mesothelin, PSMA, WT1, ROR1, CEA, GD-2, NY-ESO-1, MAGE A3, GPC3, Claudin18.2, Glycolipid F77, PD-L1, and/or PD-L2.

28. The nucleic acid of claim 27, wherein the CAR comprises the amino acid sequence set forth in SEQ ID NO: 58 or 89, or a functional variant having at least about 90% sequence identity.

29. The nucleic acid of any one of claims 23-28, wherein the nucleic acid sequence encoding the functional exogenous receptor is upstream or downstream to at least one of the one or more nucleic acid sequences encoding the fusion protein (“fusion protein nucleic acid sequence”), and optionally wherein functional exogenous receptor nucleic acid sequence and the fusion protein nucleic acid sequence are separated by a third nucleic acid sequence encoding a second multicistronic element.

30. A vector comprising the nucleic acid of any one of claims 21-29.

31. An engineered cell, comprising the fusion protein of any one of claims 1-20, the nucleic acid of claim 21 or 22, and/or the vector of claim 30.

32. The engineered cell of claim 31, further comprising a functional exogenous receptor, wherein the functional exogenous receptor comprises an extracellular ligand-binding domain and optionally an intracellular signaling domain.

33. The engineered cell of claim 32, wherein the functional exogenous receptor is selected from the group consisting of: an engineered T cell receptor (TCR), a chimeric antigen receptor (CAR), a T cell antigen coupler (TAC) or a portion thereof.

34. The engineered cell of claim 33, wherein the functional exogenous receptor specifically recognizes a tumor antigen.

35. An engineered cell, comprising the nucleic acid of any one of claims 23-29.

36. The engineered cell of any one of claims 31-35, wherein the engineered cell is an immune cell.

37. The engineered cell of claim 36, wherein the engineered cell is selected from a group consisting of T cell, NK cell, peripheral blood mononuclear cell (PBMC), hematopoietic stem cell, pluripotent stem cell, an embryonic stem cell, and a combination thereof.

38. A pharmaceutical composition comprising the engineered cell of any one of claims 31-37.

39. A method of treating a disease or condition in an individual, comprising administering to the individual the pharmaceutical composition of claim 38.

40. The method of claim 39, wherein the disease or condition is associated with immunosuppression.

41. A method of reducing an immunosuppression signal in a diseased tissue in an individual, comprising administering to the individual the pharmaceutical composition of claim 38.

42. The method of claim 41, wherein the reducing the immunosuppression signal comprises decreasing signaling through TGFβR.

43. The method of any one of claims 39-42, wherein the diseased tissue has a higher expression level of TGFβ than a corresponding tissue in an individual without the disease or condition.

44. The method of any one of claims 39-43, wherein the disease or condition is a cancer.

45. The method of claim 44, wherein the cancer is a solid tumor or liquid tumor.

46. The method of any one of claims 39-43, wherein the disease or condition is an infectious disease or a condition associated with an infection.

47. The method of any one of claims 39-46, wherein the engineered cells in the pharmaceutical composition are allogenic to the individual.

48. The method of any one of claims 39-46, wherein the engineered cells in the pharmaceutical composition are autologous to the individual.

49. The method of any one of claims 39-48, wherein the method further comprises a second therapy or administering a second agent.


SEQUENCE LISTING (90 sequences)

SEQ ID NO: 1 - protein, 33 aa, Artificial Sequence
  Synthetic Construct
MEAAVAAPRPRLLLLVLAAAAAAAAALLPGATA

SEQ ID NO: 2 - protein, 22 aa, Artificial Sequence
  Synthetic Construct
MGRGLLRGLWPLHIVLWTRIAS

SEQ ID NO: 3 - protein, 93 aa, Artificial Sequence
  Synthetic Construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 4 - protein, 144 aa, Artificial Sequence
  Synthetic Construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 5 - protein, 21 aa, Artificial Sequence
  Synthetic Construct
AAVIAGPVCFVCISLMLMVYI

SEQ ID NO: 6 - protein, 21 aa, Artificial Sequence
  Synthetic Construct
VTGISLLPPLGVAISVIIIFY

SEQ ID NO: 7 - protein, 21 aa, Artificial Sequence
  Synthetic Construct
LLLGMIVFAVMLSILSLIGIF

SEQ ID NO: 8 - protein, 25 aa, Artificial Sequence
  Synthetic Construct
WLIFFASLGSFLSILLVGVLGYLGL

SEQ ID NO: 9 - protein, 6 aa, Artificial Sequence
  Synthetic Construct
CHNRTV

SEQ ID NO: 10 - protein, 7 aa, Artificial Sequence
  Synthetic Construct
CYRVNRQ

SEQ ID NO: 11 - protein, 37 aa, Artificial Sequence
  Synthetic Construct
VPQVTSKAFQHDTWNSGLTVASISTGHLTSDNRGDIG

SEQ ID NO: 12 - protein, 3 aa, Artificial Sequence
  Synthetic Construct
VSD

SEQ ID NO: 13 - protein, 356 aa, Artificial Sequence
  Synthetic Construct
CHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 14 - protein, 380 aa, Artificial Sequence
  Synthetic Construct
CYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 15 - protein, 253 aa, Artificial Sequence
  Synthetic Construct
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 16 - protein, 92 aa, Artificial Sequence
  Synthetic Construct
NRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 17 - protein, 253 aa, Artificial Sequence
  Synthetic Construct
NRSFRTGIKRRILLLIPKWLFEDIPNMKNSNVVKMLQENSELMNNNSSEQVLFVDPMITEIKEIFIPEHKPTDFKKENTGPLETRDFPQNSLFDNTTVVFIPDLNTGFKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTFFPQNILESHFNRISLLEK

SEQ ID NO: 18 - protein, 11 aa, Artificial Sequence
  Synthetic Construct
NRSFRTGIKRR

SEQ ID NO: 19 - protein, 5 aa, Artificial Sequence
  Synthetic Construct
NRAAR

SEQ ID NO: 20 - protein, 711 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEKATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 21 - protein, 711 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEKATNFSLLKQAGDVEENPGPMEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 22 - protein, 711 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVNRAARHLCPPLPTPCASSAIEFPGGKETWQWINPVDFQEEASLQEALVVEMSWDKGERTEPLEKTELPEGAPELALDTELSLEDGDRCKAKMATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 23 - protein, 742 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEKATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 24 - protein, 742 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEKATNFSLLKQAGDVEENPGPMEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 25 - protein, 742 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 26 - protein, 742 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVPQVTSKAFQHDTWNSGLTVASISTGHLTSDNRGDIGLLLGMIVFAVMLSILSLIGIFNRSFRTGIKRRILLLIPKWLFEDIPNMKNSNVVKMLQENSELMNNNSSEQVLFVDPMITEIKEIFIPEHKPTDFKKENTGPLETRDFPQNSLFDNTTVVFIPDLNTGFKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTFFPQNILESHFNRISLLEKATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 27 - protein, 413 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVPQVTSKAFQHDTWNSGLTVASISTGHLTSDNRGDIGLLLGMIVFAVMLSILSLIGIFNRSFRTGIKRRATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVSDWLIFFASLGSFLSILLVGVLGYLGLNRAAR

SEQ ID NO: 28 - DNA, 2133 bp, Artificial Sequence
  Synthetic Construct
atggaagccgccgtggccgctcccagacctcgactgctgctgctggtgctggccgctgccgccgctgccgccgctgctctgctgcctggcgccacagccctgcagtgcttctgtcacctgtgcaccaaggacaactttacatgcgtgaccgatggcctgtgcttcgtgtccgtgacagagacaacagacaaggtgatccacaattctatgtgcatcgccgagatcgacctgatcccaagggatcgccccttcgtgtgcgcaccaagctccaagaccggaagcgtgaccacaacctactgctgtaaccaggatcactgcaataagatcgagctgccaacaaccgtgaagtctagccctggcctgggaccagtggagctggcagccgtgatcgccggccccgtgtgcttcgtgtgcatcagcctgatgctgatggtgtacatctgtcacaacaggacagtgaatcggtccttcagaaccggcatcaagcggagaatcctgctgctgatcccaaagtggctgtatgaggacatccccaacatgaagaactctaatgtggtgaagatgctgcaggagaatagcgagctgatgaacaataactcctctgagcaggtgctgtatgtggacccaatgatcacagagatcaaggagatctttatccccgagcacaagcctaccgattacaagaaggagaacacaggccccctggagacacgggactatccccagaacagcctgtttgataatacaaccgtggtgtacatccccgatctgaacaccggctataagcctcagatctccaatttcctgccagagggcagccacctgtccaataacaatgagatcacatctctgaccctgaagccccctgtggactctctggatagcggcaacaatcccagactgcagaagcaccctaatttcgccttttccgtgagctccgtgaactctctgagcaataccatcttcctgggcgagctgtctctgatcctgaaccagggcgagtgctctagccctgacatccagaatagcgtggaggaggagacaaccatgctgctggagaacgactccccttctgagacaatcccagagcagaccctgctgccagatgagttcgtgagctgtctgggcatcgtgaatgaggagctgcccagcatcaacacatactttcctcagaatatcctggagtctcacttcaacaggatcagcctgctggagaaggccaccaatttcagcctgctgaagcaggcaggagatgtggaggagaacccaggaccaatgggaaggggcctgctgaggggcctgtggcctctgcacatcgtgctgtggacacgcatcgccagcaccatcccaccccacgtgcagaagtccgtgaacaatgacatgatcgtgaccgataacaatggcgccgtgaagtttccccagctgtgcaagttctgtgacgtgcggttttccacatgcgataaccagaagtcctgcatgtctaattgtagcatcacctccatctgcgagaagcctcaggaggtgtgcgtggccgtgtggagaaagaacgacgagaatatcaccctggagacagtgtgccacgatcccaagctgccttaccacgacttcatcctggaggatgccgccagccctaagtgtatcatgaaggagaagaagaagccaggcgagacattcttcatgtgctcctgttcctctgacgagtgcaacgataatatcatcttttccgaggagtataacacatctaatcctgacctgctgctggtgatcttccaggtgaccggaatctccctgctgcctccactgggagtggccatctctgtgatcatcatcttctactgttatagggtgaaccgccagaatagggcagcaagacacctgtgcccacctctgcctacaccatgtgcaagctccgccatcgagtttccaggaggaaaggagacatggcagtggatcaaccctgtggacttccaggaggaggccagcctgcaggaggccctggtggtggagatgtcctgggataagggcgagagaacagagccactggagaagaccgagctgccagagggcgcccccgaactggcactggatactgaactgagcctggaggacggggatagatgtaaagcaaaaatg

SEQ ID NO: 29 - DNA, 2133 bp, Artificial Sequence
  Synthetic Construct
atgggaagaggactgctgagaggactgtggcccctgcatatcgtgctgtggactcgcatcgctagtacaatccccccccatgtgcagaagtccgtgaacaatgacatgatcgtgaccgataacaatggcgccgtgaagtttccccagctgtgcaagttctgtgacgtgcggttttctacatgcgataaccagaagagctgcatgtccaattgttctatcaccagcatctgcgagaagcctcaggaggtgtgcgtggccgtgtggagaaagaacgacgagaatatcaccctggagacagtgtgccacgatcccaagctgccttaccacgacttcatcctggaggatgccgcctctcctaagtgtatcatgaaggagaagaagaagccaggcgagacattcttcatgtgcagctgtagctccgacgagtgcaacgataacatcatcttttctgaggagtacaacacaagcaatcccgacctgctgctggtgatcttccaggtgaccggaatcagcctgctgccacctctgggagtggccatctccgtgatcatcatcttttactgttatagggtgaaccgccagaatcggagcttcagaaccggcatcaagcggagaatcctgctgctgatcccaaagtggctgtacgaggatatccccaacatgaagaactctaatgtggtgaagatgctgcaggagaatagcgagctgatgaacaataactctagcgagcaggtgctgtatgtggacccaatgatcacagagatcaaggagatctttatcccagagcacaagcccaccgattacaagaaggagaacacaggccccctggagacacgggactatccccagaacagcctgtttgataataccacagtggtgtacatccccgacctgaacacaggctataagcctcagatcagcaatttcctgccagagggctcccacctgtctaataacaatgagatcacatccctgaccctgaagccacccgtggacagcctggattccggcaacaatcccaggctgcagaagcaccctaatttcgccttttccgtgtcctctgtgaactctctgagcaataccatcttcctgggcgagctgtccctgatcctgaaccagggcgagtgcagctcccctgacatccagaacagcgtggaggaggagacaacaatgctgctggagaacgactccccttctgagacaatcccagagcagaccctgctgccagatgagtttgtgagctgtctgggcatcgtgaatgaggagctgccctccatcaacacatacttccctcagaatatcctggagtcccactttaaccggatctctctgctggagaaggccaccaatttctccctgctgaagcaggcaggagatgtggaggagaacccaggacctatggaggccgccgtggccgccccaaggccccgcctgctgctgctggtgctggcagccgccgcagcagcagcagccgccctgctgcctggcgccacagccctgcagtgcttctgtcacctgtgcaccaaggacaactttacatgcgtgaccgatggcctgtgcttcgtgtctgtgacagagacaacagacaaggtgatccacaatagcatgtgcatcgccgagatcgacctgatccctcgggatagacccttcgtgtgcgccccatctagcaagaccggcagcgtgaccacaacctactgctgtaaccaggatcactgcaataagatcgagctgccaacaaccgtgaagtcctctccaggcctgggacctgtggagctggcagcagtgatcgccggccccgtgtgcttcgtgtgcatctccctgatgctgatggtgtatatctgtcacaacaggacagtgaatagggcagcaaggcacctgtgccctccactgcctaccccatgtgcaagctccgccatcgagtttccaggaggaaaggagacatggcagtggatcaaccctgtggacttccaggaggaggcctccctgcaggaggccctggtggtggagatgtcttgggataagggcgagcgcacagagcctctggagaagaccgagctgccagagggcgcccccgaactggcactggatactgaactgtccctggaagacggagatagatgtaaagcaaaaatg

SEQ ID NO: 30 - DNA, 2133 bp, Artificial Sequence
  Synthetic Construct
atggaagctgctgtcgccgctccccgacctagactgctgctgctggtgctggccgccgccgctgctgccgccgccgctctgctgcccggcgccacagccctgcagtgcttctgtcacctgtgcaccaaggacaactttacatgcgtgaccgatggcctgtgcttcgtgtctgtgacagagacaacagacaaggtgatccacaatagcatgtgcatcgccgagatcgacctgatcccaagggatcgccccttcgtgtgcgcaccaagctccaagaccggaagcgtgaccacaacctactgctgtaaccaggatcactgcaataagatcgagctgccaacaaccgtgaagtctagccctggcctgggaccagtggagctggcagccgtgatcgccggccccgtgtgcttcgtgtgcatctccctgatgctgatggtgtatatctgtcacaacaggacagtgaatagggcagcaagacacctgtgcccacctctgccaaccccatgtgcatcctctgccatcgagtttcctggcggcaaggagacatggcagtggatcaacccagtggacttccaggaggaggccagcctgcaggaggccctggtggtggagatgtcctgggataagggcgagcggacagagccactggagaagaccgagctgccagagggagcacctgagctggccctggacacagagctgtccctggaggacggcgatagatgcaaggccaagatggccaccaacttttctctgctgaagcaggccggcgatgtggaggagaatcctggcccaatgggaaggggcctgctgaggggcctgtggcccctgcacatcgtgctgtggacaagaatcgcctccaccatcccaccccacgtgcagaagtctgtgaacaatgacatgatcgtgaccgataacaatggcgccgtgaagttccctcagctgtgcaagttctgtgacgtgaggtttagcacatgcgataaccagaagagctgcatgtccaattgttctatcaccagcatctgcgagaagccacaggaggtgtgcgtggccgtgtggcgcaagaacgacgagaatatcaccctggagacagtgtgccacgatcccaagctgccttaccacgactttatcctggaggatgccgcctccccaaagtgtatcatgaaggagaagaagaagcccggcgagacattcttcatgtgctcttgtagctccgacgagtgcaacgataacatcatcttttctgaggagtacaacacaagcaatcctgacctgctgctggtgatcttccaggtgaccggcatctctctgctgcctccactgggcgtggccatcagcgtgatcatcatcttttactgttatagggtgaaccgccagaatcggtccttcagaaccggcatcaagcggagaatcctgctgctgatccccaagtggctgtacgaggatatccctaacatgaagaactccaatgtggtgaagatgctgcaggagaacagcgagctgatgaacaataactctagcgagcaggtgctgtatgtggaccccatgatcacagagatcaaggagatcttcatcccagagcacaagcccaccgattacaagaaggagaacacaggccccctggagacacgggactatccccagaacagcctgtttgataatacaaccgtggtgtacatccctgacctgaacacaggctataagcctcagatctctaatttcctgccagagggctcccacctgtctaataacaatgagatcacaagcctgaccctgaagccccctgtggacagcctggattccggcaacaatcccagactgcagaagcaccctaacttcgccttttccgtgtcctctgtgaactctctgagcaataccatctttctgggcgagctgagcctgatcctgaaccagggcgagtgcagctcccccgacatccagaacagcgtggaggaggagacaaccatgctgctggagaatgactccccttctgagacaatcccagagcagaccctgctgcccgatgagttcgtgagctgtctgggcatcgtgaacgaggagctgccttccatcaatacatacttcccacagaacatcctggagtcccacttcaacagaatctcactgctggaaaag

SEQ ID NO: 31 - DNA, 2226 bp, Artificial Sequence
  Synthetic Construct
atggaagctgccgtcgctgctccccgccctcgcctgctgctgctggtcctggctgccgccgctgctgccgctgctgccctgctgcccggcgccacagccctgcagtgcttctgtcacctgtgcaccaaggacaactttacatgcgtgaccgatggcctgtgcttcgtgtctgtgacagagacaacagacaaggtgatccacaatagcatgtgcatcgccgagatcgacctgatcccaagggatcgccccttcgtgtgcgcaccaagctccaagaccggatccgtgaccacaacctactgctgtaaccaggaccactgtaataagatcgagctgcccacaaccgtgaagtctagcccaggcctgggaccagtggagctggtgcctcaggtgaccagcaaggcctttcagcacgatacatggaactccggcctgaccgtggcctctatcagcacaggccacctgacctccgacaatcggggcgatatcggcctgctgctgggcatgatcgtgttcgccgtgatgctgtctatcctgagcctgatcggcatcttcaaccggagctttagaaccggcatcaagcggagaatcctgctgctgatccccaagtggctgtacgaggacatccctaacatgaagaactctaatgtggtgaagatgctgcaggagaatagcgagctgatgaacaataactcctctgagcaggtgctgtatgtggaccccatgatcacagagatcaaggagatctttatccctgagcacaagccaaccgattacaagaaggagaacacaggccccctggagacacgggactatcctcagaactccctgtttgataatacaaccgtggtgtacatccctgatctgaacaccggctataagccacagatcagcaatttcctgcccgagggctcccacctgtctaataacaatgagatcacatccctgaccctgaagccccctgtggacagcctggattccggcaacaatcctcgcctgcagaagcacccaaatttcgcctttagcgtgagctccgtgaactccctgtctaataccatcttcctgggcgagctgtccctgatcctgaaccagggcgagtgctctagcccagacatccagaacagcgtggaggaggagacaaccatgctgctggagaacgacagcccatccgagacaatccccgagcagaccctgctgcccgatgagtttgtgagctgtctgggcatcgtgaatgaggagctgccttccatcaacacatactttccacagaatatcctggagagccacttcaaccggatctccctgctggagaaggccaccaatttctccctgctgaagcaggcaggagatgtggaggagaacccaggacctatgggaaggggcctgctgagaggcctgtggcccctgcacatcgtgctgtggacaagaatcgcctccaccatcccaccccacgtgcagaagtctgtgaacaatgacatgatcgtgaccgataacaatggcgccgtgaagttccctcagctgtgcaagttctgtgacgtgaggtttagcacatgcgataaccagaagtcttgcatgagcaattgttccatcacctctatctgcgagaagccacaggaggtgtgcgtggccgtgtggcgcaagaacgacgagaatatcaccctggagacagtgtgccacgatcctaagctgccatatcacgactttatcctggaggatgccgcctctccaaagtgtatcatgaaggagaagaagaagcccggcgagacattctttatgtgcagctgttcctctgacgagtgcaacgataatatcatcttctctgaggagtacaacaccagcaatccagacctgctgctggtgatctttcaggtgagcgattggctgatcttctttgccagcctgggctccttcctgtctatcctgctggtgggcgtgctgggatatctgggcctgaacagggcagcaaggcacctgtgccctccactgccaacaccctgtgccagctccgccatcgagtttcctggcggcaaggagacatggcagtggatcaatccagtggacttccaggaggaggcctccctgcaggaggccctggtggtggagatgtcttgggataagggcgagaggacagagcctctggagaagaccgagctgcccgagggcgcccctgaactggcactggatactgaactgagcctggaggatggagatagatgtaaagcaaagatg

SEQ ID NO: 32 - DNA, 2226 bp, Artificial Sequence
  Synthetic Construct
atgggcagggggctgctgcgggggctgtggcctctgcatattgtgctgtggactcggattgcttcaactattcctcctcatgtccagaagtctgtgaacaatgacatgatcgtgaccgataacaatggcgccgtgaagtttccccagctgtgcaagttctgtgacgtgaggtttagcacatgcgataaccagaagtcttgcatgagcaattgttccatcacctctatctgcgagaagcctcaggaggtgtgcgtggccgtgtggcgcaagaacgacgagaatatcaccctggagacagtgtgccacgatcctaagctgccataccacgactttatcctggaggatgccgccagcccaaagtgtatcatgaaggagaagaagaagcccggcgagacattcttcatgtgctcctgtagctccgacgagtgcaacgataatatcatcttcagcgaggagtataacacatccaatcctgacctgctgctggtgatctttcaggtgccacaggtgacctccaaggccttccagcacgatacatggaactctggcctgaccgtggcctctatcagcacaggccacctgacctccgacaatcggggcgatatcggcctgctgctgggcatgatcgtgtttgccgtgatgctgagcatcctgtccctgatcggcatcttcaacaggagctttcgcaccggcatcaagcggagaatcctgctgctgatccccaagtggctgtacgaggacatccctaacatgaagaacagcaatgtggtgaagatgctgcaggagaacagcgagctgatgaacaataactctagcgagcaggtgctgtatgtggacccaatgatcacagagatcaaggagatcttcatccccgagcacaagcctaccgattacaagaaggagaacacaggccccctggagacacgggactatcctcagaactccctgtttgataataccacagtggtgtacatccctgatctgaacacaggctataagccacagatctccaatttcctgcccgagggctctcacctgagcaataacaatgagatcacatctctgaccctgaagccccctgtggactccctggattctggcaacaatcctagactgcagaagcacccaaatttcgcctttagcgtgtcctctgtgaactccctgtctaataccatcttcctgggcgagctgtctctgatcctgaaccagggcgagtgcagctcccctgacatccagaatagcgtggaggaggagacaacaatgctgctggagaacgacagcccatccgagacaatccccgagcagaccctgctgccagatgagttcgtgagctgtctgggcatcgtgaatgaggagctgccttctatcaacacatacttcccacagaatatcctggagtctcactttaacaggatcagcctgctggagaaggccaccaatttcagcctgctgaagcaggcaggagacgtggaggagaaccctggaccaatggaggccgccgtggccgcccccaggcctcgcctgctgctgctggtgctggcagccgccgcagcagcagcagccgccctgctgccaggagcaacagccctgcagtgcttctgtcacctgtgcaccaaggacaactttacatgcgtgaccgatggcctgtgcttcgtgagcgtgacagagacaacagataaggtgatccacaattccatgtgcatcgccgagatcgacctgatcccacgggatagacccttcgtgtgcgccccatctagcaagaccggctccgtgaccacaacctactgctgtaaccaggaccactgtaataagatcgagctgcccacaaccgtgaagtcctctcctggcctgggaccagtggagctggtgagcgattggctgatcttctttgccagcctgggctccttcctgtctatcctgctggtgggcgtgctgggatatctgggcctgaacagggcagcaagacacctgtgcccaccactgccaacaccatgtgcaagctccgccatcgagtttccaggaggaaaggagacatggcagtggatcaatcctgtggacttccaggaggaggcctctctgcaggaggccctggtggtggagatgagctgggataagggcgagcgcacagagcccctggagaagaccgagctgcccgagggcgcccctgaactggcactggatactgaactgagcctggaagatggcgataggtgtaaagcaaaaatg

SEQ ID NO: 33 - DNA, 2226 bp, Artificial Sequence
  Synthetic Construct
atggaagccgctgtggccgctccccgacctcgactgctgctgctggtgctggctgccgctgctgctgccgctgccgctctgctgcctggcgccacagccctgcagtgcttctgtcacctgtgcaccaaggacaactttacatgcgtgaccgatggcctgtgcttcgtgagcgtgacagagacaacagacaaggtgatccacaattccatgtgcatcgccgagatcgacctgatcccaagggatcgccccttcgtgtgcgcaccaagctccaagaccggctccgtgaccacaacctactgctgtaaccaggaccactgtaataagatcgagctgcccacaaccgtgaagtctagcccaggcctgggaccagtggagctggtgagcgattggctgatcttctttgcctctctgggcagcttcctgtccatcctgctggtgggcgtgctgggatatctgggcctgaacagggcagcaagacacctgtgcccacctctgcccacaccttgtgcctcctctgccatcgagtttcctggcggcaaggagacatggcagtggatcaatccagtggacttccaggaggaggcctccctgcaggaggccctggtggtggagatgtcttgggataagggcgagaggacagagccactggagaagaccgagctgcctgagggagcaccagagctggccctggacacagagctgtctctggaggacggcgatcgctgcaaggccaagatggccaccaactttagcctgctgaagcaggccggcgatgtggaggagaatcctggaccaatgggaaggggcctgctgagaggcctgtggcctctgcacatcgtgctgtggacacggatcgcctctaccatcccaccccacgtgcagaagagcgtgaacaatgacatgatcgtgaccgataacaatggcgccgtgaagttccctcagctgtgcaagttctgtgacgtgcggtttagcacatgcgataaccagaagtcttgcatgagcaattgttccatcacctctatctgcgagaagccacaggaggtgtgcgtggccgtgtggagaaagaacgacgagaatatcaccctggagacagtgtgccacgatcctaagctgccataccacgactttatcctggaggatgccgcctctcccaagtgtatcatgaaggagaagaagaagcctggcgagacattcttcatgtgcagctgtagctccgacgagtgtaacgataatatcatcttcagcgaggagtataacacatccaatccagacctgctgctggtgatctttcaggtgccccaggtgaccagcaaggccttccagcacgatacatggaactccggcctgaccgtggcctctatcagcacaggccacctgacctccgacaataggggcgatatcggcctgctgctgggcatgatcgtgtttgccgtgatgctgagcatcctgtccctgatcggcatcttcaacaggagctttcgcaccggcatcaagcggagaatcctgctgctgatccctaagtggctgtacgaggacatcccaaacatgaagaactctaatgtggtgaagatgctgcaggagaatagcgagctgatgaacaataactctagcgagcaggtgctgtatgtggaccccatgatcacagagatcaaggagatcttcatccccgagcacaagcctaccgattacaagaaggagaacacaggccccctggagacacgggactatcctcagaactccctgtttgataatacaaccgtggtgtacatccctgatctgaacaccggctataagccacagatcagcaatttcctgcccgagggctctcacctgagcaataacaatgagatcacatccctgaccctgaagcctccagtggactccctggattctggcaacaatcctcgcctgcagaagcacccaaacttcgcctttagcgtgtcctctgtgaactccctgtctaataccatctttctgggcgagctgtccctgatcctgaaccagggcgagtgcagctcccccgacatccagaacagcgtggaggaggagacaaccatgctgctggagaatgacagcccatccgagacaatccccgagcagaccctgctgcccgatgagttcgtgtcctgtctgggcatcgtgaacgaggagctgccttctatcaatacatacttccctcagaacatcctggagagccacttcaatagaattagcctgctggagaaa

SEQ ID NO: 34 - DNA, 2226 bp, Artificial Sequence
  Synthetic Construct
atggaagctgccgtggctgctccaagacctagactgctgctgctggtgctggccgccgccgccgccgctgccgctgccctgctgcccggcgccacagccctgcagtgcttctgtcacctgtgcaccaaggacaactttacatgcgtgaccgatggcctgtgcttcgtgtctgtgacagagacaacagacaaggtgatccacaatagcatgtgcatcgccgagatcgacctgatcccaagggatcgccccttcgtgtgcgcaccaagctccaagaccggctccgtgaccacaacctactgctgtaaccaggaccactgtaataagatcgagctgcccacaaccgtgaagtctagcccaggcctgggaccagtggagctggtgcctcaggtgaccagcaaggcctttcagcacgatacatggaactccggcctgaccgtggcctctatcagcacaggccacctgacctccgacaatcggggcgatatcggcctgctgctgggcatgatcgtgttcgccgtgatgctgtctatcctgagcctgatcggcatcttcaaccggagctttagaaccggcatcaagcggagaatcctgctgctgatccccaagtggctgtttgaggacatccctaacatgaagaactctaatgtggtgaagatgctgcaggagaatagcgagctgatgaacaataactcctctgagcaggtgctgttcgtggaccccatgatcacagagatcaaggagatctttatccctgagcacaagccaaccgatttcaagaaggagaacacaggccccctggagacacgggacttccctcagaactccctgtttgataatacaaccgtggtgttcatccctgatctgaacaccggcttcaagccacagatcagcaattttctgcccgagggctcccacctgtctaataacaatgagatcacatccctgaccctgaagccccctgtggacagcctggattccggcaacaatcctcgcctgcagaagcacccaaatttcgcctttagcgtgagctccgtgaactccctgtctaataccatctttctgggcgagctgtccctgatcctgaaccagggcgagtgctctagcccagacatccagaacagcgtggaggaggagacaaccatgctgctggagaacgacagcccatccgagacaatccccgagcagaccctgctgcccgatgagttcgtgagctgtctgggcatcgtgaatgaggagctgccttccatcaacacattctttccacagaatatcctggagagccactttaaccggatctccctgctggagaaggccaccaatttctccctgctgaagcaggcaggagatgtggaggagaacccaggacctatgggaaggggcctgctgagaggcctgtggcccctgcacatcgtgctgtggacaagaatcgcctccaccatcccaccccacgtgcagaagtctgtgaacaatgacatgatcgtgaccgataacaatggcgccgtgaagttccctcagctgtgcaagttctgtgacgtgaggtttagcacatgcgataaccagaagtcttgcatgagcaattgttccatcacctctatctgcgagaagccacaggaggtgtgcgtggccgtgtggcgcaagaacgacgagaatatcaccctggagacagtgtgccacgatcctaagctgccatatcacgactttatcctggaggatgccgcctctccaaagtgtatcatgaaggagaagaagaagcccggcgagacattctttatgtgcagctgttcctctgacgagtgcaacgataatatcatcttctctgaggagtacaacaccagcaatccagacctgctgctggtgatctttcaggtgagcgattggctgatcttctttgccagcctgggctccttcctgtctatcctgctggtgggcgtgctgggatatctgggcctgaacagggcagcaaggcacctgtgccctccactgccaacaccctgtgccagctccgccatcgagtttcctggcggcaaggagacatggcagtggatcaatccagtggacttccaggaggaggcctccctgcaggaggccctggtggtggagatgtcttgggataagggcgagaggacagagcctctggagaagaccgagctgcccgagggcgcccctgagctggcactggacaccgaactgagtctggaggatggcgaccgctgtaaggctaaaatg

SEQ ID NO: 35 - DNA, 1239 bp, Artificial Sequence
  Synthetic Construct
atggaagccgccgtcgccgctccccgcccaagactgctgctgctggtcctggccgctgccgctgctgccgccgccgcactgctgcccggcgccacagccctgcagtgcttctgtcacctgtgcaccaaggacaacttcacctgcgtgaccgatggcctgtgcttcgtgtccgtgacagagacaacagacaaggtgatccacaactctatgtgcatcgccgagatcgacctgatcccaagggatcgccccttcgtgtgcgcacctagctccaagaccggctccgtgaccacaacctactgctgtaaccaggaccactgtaataagatcgagctgcccacaaccgtgaagtctagcccaggcctgggaccagtggagctggtgcctcaggtgacctctaaggccttccagcacgatacatggaatagcggcctgaccgtggccagcatctccacaggccacctgacctctgacaacaggggcgatatcggcctgctgctgggcatgatcgtgtttgccgtgatgctgtctatcctgagcctgatcggcatcttcaaccggagctttagaacaggcatcaagcggagagccaccaatttctccctgctgaagcaggcaggagatgtggaggagaaccctggaccaatgggaaggggcctgctgaggggcctgtggccactgcacatcgtgctgtggacacggatcgccagcaccatcccccctcacgtgcagaagtccgtgaacaatgacatgatcgtgaccgataacaatggcgccgtgaagtttccacagctgtgcaagttctgtgacgtgcggtttagcacatgcgataaccagaagtcctgcatgtctaattgtagcatcacctccatctgcgagaagccacaggaggtgtgcgtggccgtgtggagaaagaacgacgagaatatcaccctggagacagtgtgccacgatcctaagctgccataccacgactttatcctggaggatgccgcctctcctaagtgtatcatgaaggagaagaagaagccaggcgagacattctttatgtgcagctgttcctctgacgagtgcaacgataatatcatcttctccgaggagtataacacctctaatcccgacctgctgctggtgatctttcaggtgtccgattggctgatcttctttgcctctctgggcagcttcctgtccatcctgctggtgggggtcctgggctacctggggctgaatcgggctgctcgg

SEQ ID NO: 36 - protein, 491 aa, Artificial Sequence
  Synthetic Construct
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 1257 aa, Artificial Sequence
  Synthetic Construct
MALPVTALLLPLALLLHAARPDIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRVNGSGATNFSLLKQAGDVEENPGPMEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMATNFSLLKQAGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 38 - DNA, 3771 bp, Artificial Sequence
  Synthetic Construct
atggccctgcctgtgaccgctctgctgctgcctctggctctgctgctgcacgctgcccgccctgacattgtgatgacccagtctcccctgtccctgccagtgacccctggagagccagcctccatctcttgcaggagctcccagaccatcgtgcacagcaacggcaatacatacctggagtggtatctgcagaagcccggccagtcccctcagctgctgatctacaaggtgtctaacaggttcagcggagtgccagaccgctttagcggatccggatctggcaccgatttcacactgaagatctctagagtggaggccgaggacgtgggcgtgtactattgcttccaggtgagccacgtgccatatacctttggcggcggcacaaagctggagatcaagggcagcacctccggatctggcaagccaggatccggagagggatctacaaagggacaggtgcagctggtgcagagcggagcagaggtgaagaagcctggagccagcgtgaaggtgtcctgtaaggcctctggctacaccttcacagactatgagatgcactgggtgcggcaggcaaccggacagggcctggagtggatgggaggaatcgacccagagacaggcaacacagcctactcccagaagtttaagggccgggtgaccatgacaagaaataccagcatctccacagcctatatggagctgtctagcctgagatctgaggatacagccgtgtactattgtgctcgctactattcctttgcctactggggacaggggacactggtcaccgtctcatcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttaacggcagcggagctacaaacttcagtcttctaaaacaggctggtgacgtggaggagaatcccggccctatggaggccgccgtggcggctcctcggccccgcctcctcctgctggtgctggccgcagctgctgccgccgcggctgccttgctgcctggcgccaccgcgctgcagtgcttttgtcacctatgcactaaggacaacttcacctgcgtgaccgacggcctgtgcttcgtgtccgtaacagagactaccgataaagtgattcacaactccatgtgcatcgccgagattgacctgatcccgcgcgaccgccccttcgtctgtgctccctcatccaagaccggcagcgtcaccacaacttactgctgcaaccaggaccactgtaacaagatcgagctgcccacaacagtcaaatcctctcctggattgggacccgtggagctggtgagtgactggttgatcttctttgcgagcctaggctccttcttaagcatccttcttgtgggcgtcctgggttacctgggactgaaccgcgctgcgcgccacctgtgcccgcctctgccaaccccctgcgcctcctcggccatcgagttccccggggggaaggagacctggcaatggatcaatccagtagactttcaggaggaggcatcgctgcaggaggctctggtggtggagatgagttgggacaagggtgaacggaccgaaccactcgagaaaaccgagctgccagaaggtgcgccggagctggccctggatactgagctttcactggaggacggcgacaggtgtaaggccaaaatggccaccaactttagcctgctgaaacaggccggtgatgtggaggagaatccgggcccaatgggccgcggcctcctacgtggactgtggcccttgcacatcgtgctttggacccgcatcgcttctaccatcccacctcatgtgcagaagagcgttaataacgacatgatcgtcactgataacaacggggcggtgaagttccctcagctgtgcaagttctgtgatgtacgcttctctacttgcgacaaccagaagagttgtatgtctaactgctctattacttctatctgcgagaagccccaggaggtctgcgtggccgtttggaggaagaatgacgagaacatcactctggagacagtatgccatgatccgaagctgccctatcatgacttcatcctggaggatgccgcaagccctaagtgcatcatgaaggagaagaagaagccgggggagaccttcttcatgtgctcctgctcttccgatgagtgcaacgacaacatcatcttttccgaggagtacaacacttccaaccccgatctgctgttggtgattttccaggtgccgcaggtcacctccaaggcgttccagcatgacacctggaacagcggactaaccgtggctagcatcagcaccggccacttgacctccgacaaccgcggggacatcggcctgctactcggaatgatcgtgttcgcggtgatgctctccatcctctctctcatcggcatcttcaaccgcagcttccgcaccggtattaagaggagaattctgcttctcatccccaaatggttatacgaggacatccccaacatgaaaaacagcaacgtggtcaagatgctgcaggagaattcggagctgatgaacaataactcatcggagcaggttctgtacgtggacccgatgatcacggaaataaaggagatcttcatccctgagcacaagcctaccgactacaagaaagagaataccggcccgcttgagactcgtgactatcctcagaactctctgttcgacaacacgacggtggtgtacattcccgacttgaacacgggctacaaaccccaaatttccaacttcctgcctgagggcagtcacctgtccaacaacaacgaaatcacctccctgaccctgaagccaccggtcgactccttggacagcggcaacaacccccgactccagaagcaccccaattttgctttttccgtgtcctccgtcaactccctctcaaataccatcttcctcggcgagctctccctgatcctgaatcagggcgaatgttcgtccccggacatccagaactctgtggaggaggagaccacgatgcttctggagaacgacagcccgtccgagaccatccccgagcagacgcttctgcccgacgagttcgtgagttgcctgggcattgtgaacgaggaactgccttctattaacacctacttccctcagaacatactggagagccactttaaccgtatctcgctattggagaag

SEQ ID NO: 39 - protein, 21 aa, Artificial Sequence
  Synthetic Construct
MALPVTALLLPLALLLHAARP

SEQ ID NO: 40 - protein, 45 aa, Artificial Sequence
  Synthetic Construct
TTTPAPRPPTPAPTIASQPLSLRPEACRPAAGGAVHTRGLDFACD

SEQ ID NO: 41 - protein, 24 aa, Artificial Sequence
  Synthetic Construct
IYIWAPLAGTCGVLLLSLVITLYC

SEQ ID NO: 42 - protein, 42 aa, Artificial Sequence
  Synthetic Construct
KRGRKKLLYIFKQPFMRPVQTTQEEDGCSCRFPEEEEGGCEL

SEQ ID NO: 43 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 6 bp, Artificial Sequence
  Synthetic Construct
gaattc

SEQ ID NO: 45 - DNA, 6 bp, Artificial Sequence
  Synthetic Construct
actagt

SEQ ID NO: 46 - DNA, 6 bp, Artificial Sequence
  Synthetic Construct
gttaac

SEQ ID NO: 47 - DNA, 6 bp, Artificial Sequence
  Synthetic Construct
acgcgt

SEQ ID NO: 48 - protein, 19 aa, Artificial Sequence
  Synthetic Construct
ATNFSLLKQAGDVEENPGP

SEQ ID NO: 49 - protein, 406 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 50 - protein, 286 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 51 - protein, 245 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 52 - protein, 447 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 53 - protein, 437 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 54 - protein, 286 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 55 - protein, 477 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 56 - protein, 246 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 57 - protein, 15 aa, Artificial Sequence
  Synthetic Construct
SGSGKPGSGEGSTKG

SEQ ID NO: 58 - protein, 245 aa, Artificial Sequence
  Synthetic Construct
DIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIKGSTSGSGKPGSGEGSTKGQVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSS

SEQ ID NO: 59 - DNA, 9 bp, Artificial Sequence
  Synthetic Construct
gccgccacc

SEQ ID NO: 60 - protein, 22 aa, Artificial Sequence
  Synthetic Construct
GSGATNFSLLKQAGDVEENPGP

SEQ ID NO: 61 - protein, 1 aa, Artificial Sequence
  Synthetic Construct
  VARIANT 1: Can be present in repeats of any integer
G

SEQ ID NO: 62 - protein, 2 aa, Artificial Sequence
  Synthetic Construct
  VARIANT (1)..(2): Can be present in repeats of any integer
GS

SEQ ID NO: 63 - protein, 5 aa, Artificial Sequence
  Synthetic Construct
  VARIANT (1)..(5): Can be present in repeats of any integer
GSGGS

SEQ ID NO: 64 - protein, 5 aa, Artificial Sequence
  Synthetic Construct
  VARIANT (1)..(5): Can be present in repeats of any integer
GGGGS

SEQ ID NO: 65 - protein, 4 aa, Artificial Sequence
  Synthetic Construct
  VARIANT (1)..(4): Can be present in repeats of any integer
GGGS

SEQ ID NO: 66 - protein, 503 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 67 - protein, 507 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGPFSVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 68 - protein, 95 aa, Artificial Sequence
  Synthetic Construct
TTDKVIHNSMCIAEIDLIPRDRPFVCAPSSKTGSVTTTYCCNQDHCNKIELPTTVKSSPGLGPVELAAVIAGPVCFVCISLMLMVYICHNRTVIH

SEQ ID NO: 69 - protein, 426 aa, Artificial Sequence
  Synthetic Construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 70 - protein, 567 aa, Artificial Sequence
  Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 71 - protein, 592 aa, Artificial Sequence
  Synthetic Construct
MGRGLLRGLWPLHIVLWTRIASTIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDLLLVIIQVTGVSLLPPLGIAIAVIIIFYCYRVHRQQKLSPSWESSKPRKLMDFSDNCAIILEDDRSDISSTCANNINHNTELLPIELDTLVGKGRFAEVYKAKLKQNTSEQFETVAVKIFPYEEYSSWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDSVLRDRGRPEIPSFWLNHQGIQIVCETLTECWDHDPEARLTAQCVAERFSELEHPERLSGRSCSQEKIPEDGSLNTTK

SEQ ID NO: 72 - protein, 592 aa, Artificial Sequence
  Synthetic Construct
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 73 - protein, 629 aa, Artificial Sequence [sequence omitted]

SEQ ID NO: 74 - protein, 375 aa, Artificial Sequence
  Synthetic Construct
MRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVAYIPDLDTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 75 - protein, 264 aa, Artificial Sequence
  Synthetic Construct
MEFWANSCFHLYRAPYFWIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPRETIPEQTLLPDEFVSCLGIVNEELPSINTYFPQNILESHFNRISLLEK

SEQ ID NO: 76 - protein, 374 aa, Artificial Sequence
  Synthetic Construct
MILRPYQPCGTVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 77 - protein, 227 aa, Artificial Sequence
  Synthetic Construct
MKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 78 - protein, 662 aa, Artificial Sequence
  Synthetic Construct
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 79 - protein, 660 aa, Artificial Sequence
  Synthetic Construct
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCDR

SEQ ID NO: 80 - protein, 115 aa, Artificial Sequence
  Synthetic Construct
QVQLVQSGAEVKKPGASVKVSCKASGYTFTDYEMHWVRQATGQGLEWMGGIDPETGNTAYSQKFKGRVTMTRNTSISTAYMELSSLRSEDTAVYYCARYYSFAYWGQGTLVTVSS

SEQ ID NO: 81 - protein, 112 aa, Artificial Sequence
  Synthetic Construct
DIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQVSHVPYTFGGGTKLEIK

SEQ ID NO: 82 - protein, 5 aa, Artificial Sequence
  Synthetic Construct
DYEMH

SEQ ID NO: 83 - protein, 17 aa, Artificial Sequence
  Synthetic Construct
GIDPETGNTAYSQKFKG

SEQ ID NO: 84 - protein, 6 aa, Artificial Sequence
  Synthetic Construct
YYSFAY

SEQ ID NO: 85 - protein, 16 aa, Artificial Sequence
  Synthetic Construct
RSSQTIVHSNGNTYLE

SEQ ID NO: 86 - protein, 7 aa, Artificial Sequence
  Synthetic Construct
KVSNRFS

SEQ ID NO: 87 - protein, 9 aa, Artificial Sequence
  Synthetic Construct
FQVSHVPYT

SEQ ID NO: 88 - protein, 567 aa, Artificial Sequence
  Synthetic Construct
MGRGLLRGLWPLHIVLWTRIASTIPPHVPKSVNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDLLLVIIQVTGVSLLPPLGIAIAVIIIFYCYRVHRQQKLSPSWESSKPRKLMDFSDNCAIILEDDRSDISSTCANNINHNTELLPIELDTLVGKGRFAEVYKAKLKQNTSEQFETVAVKIFPYEEYSSWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDSVLRDRGRPEIPSFWLNHQGIQIVCETLTECWDHDPEARLTAQCVAERFSELEHPERLSGRSCSQEKIPEDGSLNTTK

SEQ ID NO: 89 - protein, 518 aa, Artificial Sequence
  Synthetic Construct
MALPVTALLLPLALLLHAARPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGENLYFQSGGDTQALLRNDQVYQPLRDRDDAQYSHLGGNGGSGERPPPVPNPDYEPIRKGQRDLYSGLNQRGGSGDKQTLLPNDQLYQPLKDREDDQYSHLQGNGGSGRKQRITETESPYQELQGQRSDVYSDLNTQGGSG

SEQ ID NO: 90 - DNA, 2835 bp, Artificial Sequence
  Synthetic Construct
atgggctccagcaactccaagaggcagcaacagggcttgctcaagctctggcgagggctgcgagggaagcctggggcagactgggtgctattgtccgatccgcttatcgggcagtcatcaacagtccaagaagagtgcggcaaggccttgaaaaagtcctggggtaaaggtaaaatgactccagacggccgccgcctgcaagaaggagacacctttgatgagtgggatgatgatgaagaagaagtaggcttccctgtgcaacctcgagtccccttaagacagatgacctataaattagcagtggacttttcccactttttaaaatcaaaggggggactggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtgctgcacatccagcacagatgggggaagatcctgatggaatagatcatggagaagtcttggtctggaagtttgacccgaagttggcggtggagtaccgcccggacatgtttaaggacatgcacgaacatgcaaagcgctgaacgcgtgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaggatccgccgccaccatggccctgccagtgaccgccttgctccttcccctggctcttctgctgcacgctgctagacctgaggtgcagctgcagcagagcggagctgagctggtgaagcctggcgctagcgtgaagatgagctgcaaggccagcggctacaccttcaccagctataacatgcactgggtgaagcagacccctggacagggactggagtggatcggagctatctaccctggaaacggagacacctcatacaaccagaagttcaagggaaaggctaccctgaccgctgacaagagcagcagcaccgcttacatgcagctgagctcactgaccagcgaggactccgccgactactactgcgccagaagcaactactacggaagcagctactggttcttcgacgtgtggggagctggaaccaccgtgaccgtgtcaagcggcggcggaggctccggaggcggaggatctggcggcggcggcagcgacatcgtgctgacccagagccctgctatcctgtctgccagccctggagagaaggtgaccatgacctgcagagctagcagcagcgtgaactacatggactggtatcagaaaaagcccggcagctcacctaagccttggatctacgctaccagcaacttagccagcggcgtgcctgctagattctccggaagcggctctggaaccagctactcccttaccatcagcagagtggaggctgaggacgctgctacctactactgccagcagtggagcttcaaccctcctaccttcggaggaggaaccaagctggagatcaagactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgggtgaaaatttgtattttcaatctggtggtgacacacaagctctgttgaggaatgaccaggtctatcagcccctccgagatcgagatgatgctcagtacagccaccttggaggaaacggtggttctggtgagaggccaccacctgttcccaacccagactatgagcccatccggaaaggccagcgggacctgtattctggcctgaatcagagaggtggttctggtgacaagcagactctgttgcccaatgaccagctctaccagcccctcaaggatcgagaagatgaccagtacagccaccttcaaggaaacggtggttctggtcggaaacagcgtatcactgagaccgagtcgccttatcaggagctccagggtcagaggtcggatgtctacagcgacctcaacacacagggtggttctggttaa